(19) United States
(12) Patent Application Publication
(10) Pub. No.: US 2025/0206743 A1
(43) Pub. Date: Jun. 26, 2025

(54) TYK2 INHIBITOR SYNTHESIS AND INTERMEDIATES THEREOF

(71) Applicant: Takeda Pharmaceutical Company Limited, Osaka (JP)

(72) Inventors: Craig E. MASSE, Cambridge, MA (US); Avinash S. PHADKE, Branford, CT (US); Jon P. LAWSON, Boston, MA (US); Stuart LEVY, Arlington, MA (US); Xiaowei YANG, Beijing (CN); Guisheng WU, Beijing (CN); Shufeng FAN, Tianjin (CN)

(21) Appl. No.: 18/850,475

(22) PCT Filed: Mar. 24, 2023
(86) PCT No.: PCT/US2023/064914

§ 371 Date: Sep. 24, 2024

Related U.S. Application Data
(60) Provisional application No. 63/269,946, filed on Mar. 25, 2022.

(51) Int. Cl. C07D 487/04

(52) U.S. Cl. CPC C07D 487/04

(57) ABSTRACT

Described herein are methods of synthesis of a tyrosine-protein kinase 2 (TYK2) inhibitor and to intermediate compounds of the synthesis and methods of making the intermediates. Also provided are pharmaceutically acceptable compositions including compounds prepared by the synthetic method and methods of treating disorders using the same.

### CLAIM OF PRIORITY

This application claims priority to U.S. Provisional Application No. 63/269,946, filed Mar. 25, 2022, the entire contents of which are hereby incorporated by reference.

### TECHNICAL FIELD

The present disclosure relates to methods of synthesis of a tyrosine-protein kinase 2 (TYK2) inhibitor and to intermediate compounds of the synthesis and methods of making the intermediates. Also provided are pharmaceutically acceptable compositions including compounds prepared by the synthetic method and methods of treating disorders using the same.

### BACKGROUND

Protein kinases constitute a large family of structurally related enzymes that are responsible for the control of a variety of signal transduction processes within the cell. Protein kinases are thought to have evolved from a common ancestral gene due to the conservation of their structure and catalytic function. Almost all kinases contain a similar 250-300 amino acid catalytic domain. The kinases may be categorized into families by the substrates they phosphorylate (e.g., protein-tyrosine, protein-serine/threonine, lipids, etc.).

In general, protein kinases mediate intracellular signaling by effecting a phosphoryl transfer from a nucleoside triphosphate to a protein acceptor that is involved in a signaling pathway. These phosphorylation events act as molecular on/off switches that can modulate or regulate the target protein biological function. These phosphorylation events are ultimately triggered in response to a variety of extracellular and other stimuli. Examples of such stimuli include environmental and chemical stress signals (e.g., osmotic shock, heat shock, ultraviolet radiation, bacterial endotoxins, and H₂O₂), cytokines (e.g., interleukin-1 (IL-1), interleukin-8 (IL-8), and tumor necrosis factor α (TNF-α)), and growth factors (e.g., granulocyte macrophage-colony-stimulating factor (GM-CSF), and fibroblast growth factor (FGF)). An extracellular stimulus may affect one or more cellular responses related to cell growth, migration, differentiation, secretion of hormones, activation of transcription factors, muscle contraction, glucose metabolism, control of protein synthesis, and regulation of the cell cycle.

Many diseases are associated with abnormal cellular responses triggered by kinase-mediated events. These diseases include, but are not limited to, autoimmune diseases, inflammatory diseases, bone diseases, metabolic diseases, neurological and neurodegenerative diseases, cancer, cardiovascular diseases, allergies and asthma, Alzheimer's disease, and hormone-related diseases.

TYK2 catalyzes the phosphorylation of STAT proteins downstream of a number of cytokine receptors, including the Type I interferon receptor and the IL-12 and IL-23 receptors. The activation of TYK2-dependent receptors by their cytokine ligands results in the activation of STAT-dependent transcription and cellular functional responses specific for the receptors and cell types on which they are expressed. The cytokine signaling pathways regulated by TYK2 play key roles in several immune-mediated disorders. The cytokine IL-12 is essential for the development of Type 1 T-helper cells (Th1) which produce interferon-gamma, a major effector molecule in systemic autoimmune disorders such as systemic lupus erythematosus. The cytokine IL-23 is central for the expansion and survival of Th17 cells and innate lymphoid cells, both of which have been shown to play key pathogenic roles in autoimmunity. IL-23 stimulation drives the production of key proinflammatory cytokines by Th17 cells, including IL-17A, IL-17F, and IL-22, all of which are effector molecules important for pathogenesis of conditions such as psoriasis, psoriatic arthritis, and spondyloarthritis. Inhibition of TYK2 would be expected to impact multiple immune-mediated disorders through its effects on the IL-23/Th17/Th22 axis, IL-12-mediated Th1 functions, and Type I interferon-driven modulation of diverse immune pathways and cell types.

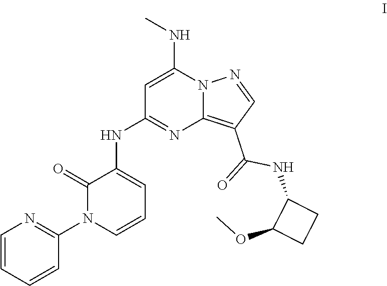 [SMILES: CNc1cc(Nc2cccn(-c3ccccn3)c2=O)nc2c(C(=O)N[C@@H]3CC[C@H]3OC)cnn12]

is a TYK2 inhibitor. See U.S. Pat. No. 11,046,698. There remains a need for synthetic methods that can efficiently provide large quantities (e.g., kilogram scale or greater) of compound 1 for use in further clinical research and treatment. The present disclosure satisfies this need and provides other related advantages.

### SUMMARY

In one aspect, Compound 1:

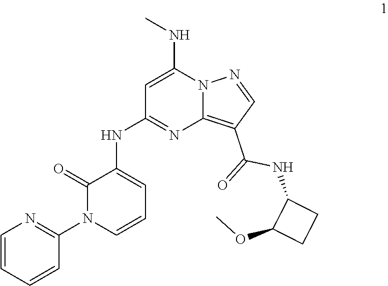 [SMILES: CNc1cc(Nc2cccn(-c3ccccn3)c2=O)nc2c(C(=O)N[C@@H]3CC[C@H]3OC)cnn12]

or a pharmaceutically acceptable salt or solvate thereof, produced by a method disclosed herein, is described herein.

In one aspect, a method for preparing Compound 1:

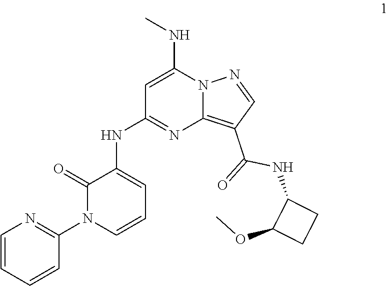 [SMILES: CNc1cc(Nc2cccn(-c3ccccn3)c2=O)nc2c(C(=O)N[C@@H]3CC[C@H]3OC)cnn12]

or a pharmaceutically acceptable salt or solvate thereof, is described herein.

In another aspect, methods for preparing a compound of formula I:

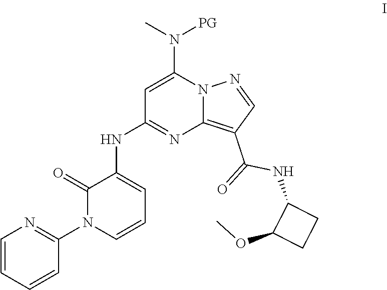

or a pharmaceutically acceptable salt thereof, wherein PG is a suitable amino protecting group as defined herein, are described herein.

In another aspect, methods for preparing a compound of formula II:

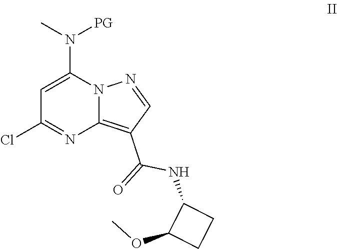

or a pharmaceutically acceptable salt thereof, wherein PG is a suitable amino protecting group as defined herein, are described herein.

In one aspect, a compound of Formula I:

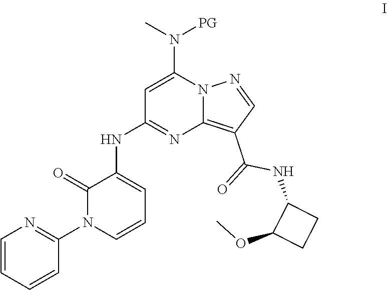

or a pharmaceutically acceptable salt thereof, wherein:
PG is a suitable amino protecting group, is described herein.

In one aspect, a compound of Formula II:

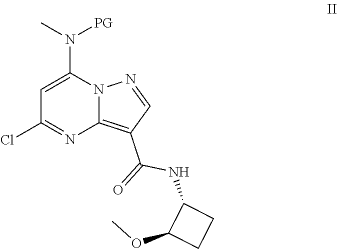

or a pharmaceutically acceptable salt thereof, wherein:
PG is a suitable amino protecting group as defined herein, is described herein.

In another aspect, a method of inhibiting a TYK2 protein kinase, or a mutant thereof, in a patient is described; the method including administering to the patient a therapeutically effective amount of Compound 1:

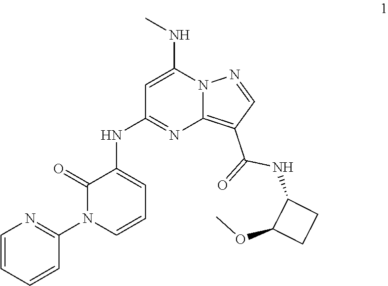 [SMILES: CNc1cc(Nc2cccn(-c3ccccn3)c2=O)nc2c(C(=O)N[C@@H]3CC[C@H]3OC)cnn12]

or a pharmaceutically acceptable salt or solvate thereof, wherein Compound 1 is produced by the methods described herein.

Other aspects, embodiments, and features will be apparent from the following description, the drawings, and the claims.

### DETAILED DESCRIPTION

Compound 1, described below, is an oral, allosteric selective TYK2 inhibitor for the treatment of psoriasis, psoriatic arthritis and other inflammatory and autoimmune diseases. There are currently no TYK2 inhibitors approved, and Compound 1 is the most selective TYK2 inhibitor currently in clinical development. Furthermore, Compound 1's selectivity and potential to provide higher levels of TYK2 inhibition for a longer period with once-daily (QD) dosing may confer clinical and ultimately commercial advantages over other TYK2 inhibitors in development. TYK2 is a member of the Janus kinase (JAK) family of kinases, a class of intracellular signaling proteins that regulate chronic inflammation in inflammatory and autoimmune diseases. Although JAK inhibition can be effective in treating inflammatory and autoimmune diseases, it also can produce on-target safety issues by modulating a broad variety of cytokine pathways. As a result, while JAK inhibitors have become established oral treatments for numerous inflammatory and autoimmune diseases, their clinical utility is constrained by elevated risk of infections and other side effects that have resulted in U.S. Food and Drug Administration (FDA)-mandated boxed warnings and dosing limitations as part of their labeling. Designing selective JAK inhibitors that directly and specifically inhibit the intended kinase function is challenging due to the structural similarity between the catalytic (orthosteric or JH1) sites for drug targeting on the JAK catalytic domains. Based on human genetic data and growing clinical evidence for the selectivity of allosteric TYK2 inhibitors, the present approach of selective allosteric inhibition of TYK2 provides an optimal balance of achieving strong efficacy while potentially avoiding safety concerns associated with broader JAK inhibition for the treatment of multiple inflammatory and autoimmune diseases. Accordingly, robust, scalable, and enantioselective synthetic procedures to produce compound for further testing is important for the use of this compound in research and in clinical settings.

U.S. Pat. No. 11,046,698, the entirety of which is hereby incorporated herein by reference, describes certain therapeutically beneficial compounds. Such compounds include Compound 1:

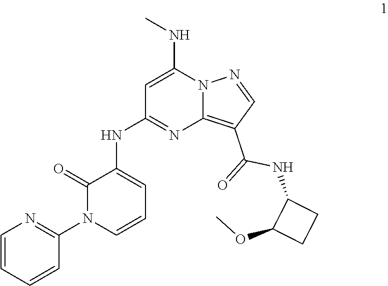 [SMILES: CNc1cc(Nc2cccn(-c3ccccn3)c2=O)nc2c(C(=O)N[C@@H]3CC[C@H]3OC)cnn12]

or a pharmaceutically acceptable salt thereof.

Compound 1 is designated as I-908 in U.S. Pat. No. 11,046,698 and an alternative synthesis of compound 1, which is different from the improved synthesis described herein, is described in detail at Example 40 of U.S. Pat. No. 11,046,698. It would be desirable to provide improved methods of synthesizing compound 1, or a pharmaceutically acceptable salt or solvate thereof. Accordingly, methods of synthesizing such compounds and their pharmaceutically acceptable salts are described herein.

In some embodiments, improved methods of preparing Compound 1, or a pharmaceutically acceptable salt or solvate thereof, are described wherein such methods produce Compound 1 in higher yield, fewer steps, milder conditions, and/or with greater generality (greater structural variation of the desired compounds). In some embodiments, as described further herein, a method of preparing Compound 1 or a pharmaceutically acceptable salt or solvate thereof is described.

In another aspect, intermediates useful in preparing a Compound 1 are provided. Such intermediates include those described in detail below.

In another aspect, described herein is Compound 1, or a pharmaceutically acceptable salt or solvate thereof, characterized in that the compound is prepared according to a method of synthesis described herein. In another aspect, a pharmaceutical composition including Compound 1, or a pharmaceutically acceptable salt or solvate thereof, is described, characterized in that the compound is prepared according to a method of synthesis described herein.

Compounds prepared by the methods herein, and pharmaceutically acceptable salts and pharmaceutical compositions thereof, are useful for treating, preventing, ameliorating, or promoting recovery from a variety of injuries, diseases, and disorders, including those described herein.

### Compounds and Definitions

Compounds include those described generally herein, and are further illustrated by the classes, subclasses, and species disclosed herein. As used herein, the following definitions shall apply unless otherwise indicated. For purposes of this disclosure, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

The term “aliphatic” or “aliphatic group,” as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle” or “cycloaliphatic”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

As used herein, the term “bicyclic ring” or “bicyclic ring system” refers to any bicyclic ring system, i.e. carbocyclic or heterocyclic, saturated or having one or more units ofunsaturation, having one or more atoms in common between the two rings of the ring system. Thus, the term includes any permissible ring fusion, such as ortho-fused or spirocyclic. As used herein, the term “heterobicyclic” is a subset of “bicyclic” that requires that one or more heteroatoms are present in one or both rings of the bicycle. Such heteroatoms may be present at ring junctions and are optionally substituted, and may be selected from nitrogen (including N-oxides), oxygen, sulfur (including oxidized forms such as sulfones and sulfonates), phosphorus (including oxidized forms such as phosphates), boron, etc. In some embodiments, a bicyclic group has 7-12 ring members and 0-4 heteroatoms independently selected from nitrogen, oxygen, or sulfur. As used herein, the term “bridged bicyclic” refers to any bicyclic ring system, i.e. carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, or sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally or alternatively, any substitutable nitrogen of a bridged bicyclic group is optionally substituted. Exemplary bicyclic rings include:

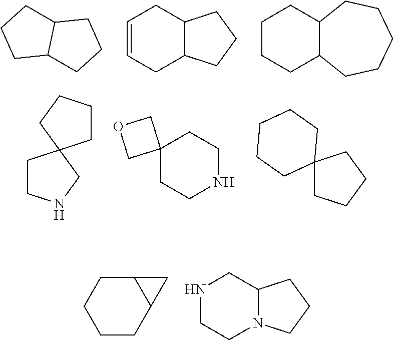

Exemplary bridged bicyclics include:

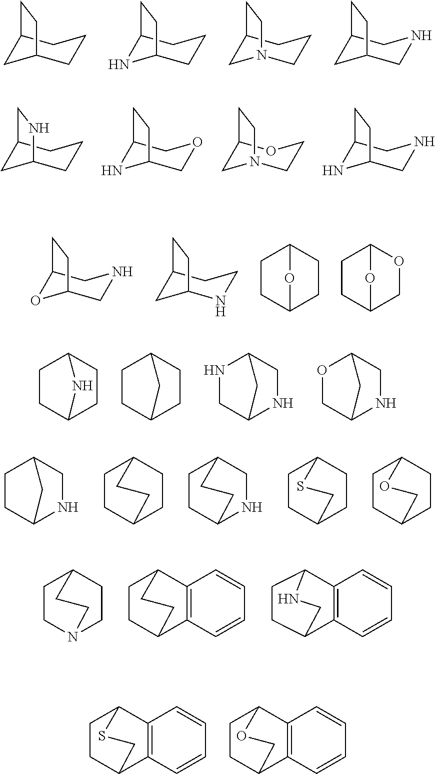

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated,” as used herein, means that a moiety has one or more units of unsaturation.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A “substituted” alkylene chain is a polymethylene group in which one or more methylene hydrogen atoms are replaced with a substituent. Suitable substituents include those described below for a substituted aliphatic group.

The term “alkenylene” refers to a bivalent alkenyl group having at least one carbon-carbon double bond. Unless otherwise specified, the double bond may be cis or trans. In some embodiments, an alkenylene group has a single carbon-carbon double bond. In some embodiments, the double bond is cis. In some embodiments, the double bond is trans. A substituted alkenylene chain is a polymethylene group containing at least one double bond in which one or more hydrogen atoms are replaced with a substituent. Suitable substituents include those described below for a substituted aliphatic group.

The term “alkynylene” refers to a bivalent alkynyl group having at least one carbon-carbon triple bond. A carbon-carbon triple bond may be located at an internal or terminal location in the alkynylene group, i.e., at either end or between two carbon atoms internal to the chain or carbon atoms. A substituted alkynylene chain is a polymethylene group containing at least one triple bond in which one or more hydrogen atoms are replaced with a substituent. Suitable substituents include those described below for a substituted aliphatic group. In some embodiments, the triple bond is at the terminal position and the alkynyl hydrogen is optionally replaced by a substituent.

The term “halogen” means F, Cl, Br, or I.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle,” “heterocyclyl,” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl, pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be mono- or bicyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Each optional substituent on a substitutable carbon is a monovalent substituent independently selected from halogen; —(CH₂)₀₋₄Rº; —(CH₂)₀₋₄ORº; —O(CH₂)₀₋₄Rº, —O—(CH₂)₀₋₄C(O)ORº; —(CH₂)₀₋₄CH(ORº)₂; —(CH₂)₀₋₄SRº; —(CH₂)₀₋₄Ph, which may be substituted with Rº; —(CH₂)₀₋₄O(CH₂)₀₋₁Ph which may be substituted with Rº; —CH═CHPh, which may be substituted with Rº; —(CH₂)₀₋₄O(CH₂)₀₋₁-pyridyl which may be substituted with Rº; —NO₂; —CN; —N₃; —(CH₂)₀₋₄N(Rº)₂; —(CH₂)₀₋₄N(Rº)C(O)Rº; —N(Rº)C(S)Rº; —(CH₂)₀₋₄N(Rº)C(O)NRº₂; —N(Rº)C(S)NRº₂; —(CH₂)₀₋₄N(Rº)C(O)ORº; —N(Rº)N(Rº)C(O)Rº; —N(Rº)N(Rº)C(O)NRº₂; —N(Rº)N(Rº)C(O)ORº; —(CH₂)₀₋₄C(O)Rº; —C(S)Rº; —(CH₂)₀₋₄C(O)ORº; —(CH₂)₀₋₄C(O)SRº; —(CH₂)₀₋₄C(O)OSiRº₃; —(CH₂)₀₋₄OC(O)Rº; —OC(O)(CH₂)₀₋₄SR—, SC(S)SRº; —(CH₂)₀₋₄SC(O)Rº; —(CH₂)₀₋₄C(O)NRº₂; —C(S)NRº₂; —C(S)SRº; —SC(S)SRº, —(CH₂)₀₋₄OC(O)NRº₂; —C(O)N(ORº)Rº; —C(O)C(O)Rº; —C(O)CH₂C(O)Rº; —C(NORº)Rº; —(CH₂)₀₋₄SSRº; —(CH₂)₀₋₄S(O)₂Rº; —(CH₂)₀₋₄S(O)₂ORº; —(CH₂)₀₋₄OS(O)₂Rº; —S(O)₂NRº₂; —(CH₂)₀₋₄S(O)Rº; —N(Rº)S(O)₂NRº₂; —N(Rº)S(O)₂Rº; —N(ORº)Rº; —C(NH)NRº₂; —P(O)₂Rº; —P(O)Rº₂; —OP(O)Rº₂; —OP(O)(ORº)₂; SiRº₃; —(C₁₋₄ straight or branched alkylene)O—N(Rº)₂; or —(C₁₋₄ straight or branched alkylene)C(O)O—N(Rº)₂.

Each Rº is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of Rº, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted by a divalent substituent on a saturated carbon atom of Rº selected from ═O and ═S; or each Rº is optionally substituted with a monovalent substituent independently selected from halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{●} 3, —OSiR^{●} 3, —C(O)SR^{●}, —(C₁₋₄ straight or branched alkylene)C(O)OR^{●}, or —SSR^{●}.

Each R^{●} is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, and wherein each R^{●} is unsubstituted or where preceded by halo is substituted only with one or more halogens; or wherein an optional substituent on a saturated carbon is a divalent substituent independently selected from ═O, ═S, ═NNR^{●}₂, ═NNHC(O)R*, ═NNHC(O)OR*, ═NNHS(O)₂R*, ═NR*, ═NOR*, —O(C(R^{●}₂))₂₋₃O—, or —S(C(R*₂))₂₋₃S—, or a divalent substituent bound to vicinal substitutable carbons of an “optionally substituted” group is —O(CR*₂)₂₋₃O—, wherein each independent occurrence of R* is selected from hydrogen, C₁₋₆ aliphatic or an unsubstituted 5-6-membered saturated, partially unsaturated, or aryl ring having 0-4 heteroatoms independently selected from nitrogen, oxygen, or sulfur.

When R* is C₁₋₆ aliphatic, R* is optionally substituted with halogen, —R^{●}, -(haloR^{●}), —OH, —OR^{●}, —O(haloR^{●}), —CN, —C(O)OH, —C(O)OR^{●}, —NH₂, —NHR^{●}, —NRº₂, or —NO₂, wherein each R^{●} is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, and wherein each R^{●} is unsubstituted or where preceded by halo is substituted only with one or more halogens.

An optional substituent on a substitutable nitrogen is independently —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic, unsubstituted —OPh, or an unsubstituted 5-6-membered saturated, partially unsaturated, or aryl ring having 0-4 heteroatoms independently selected from nitrogen, oxygen, or sulfur, or, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur; wherein when R^{†} is C₁₋₆ aliphatic, RT is optionally substituted with halogen, —R^{●}, -(haloR^{●}), —OH, —OR^{●}, —O(haloR^{●}), —CN, —C(O)OH, —C(O)OR^{●}, —NH₂, —NI—R, —NR*₂, or —NO₂, wherein each R^{●} is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur, and wherein each R^{●} is unsubstituted or where preceded by halo is substituted only with one or more halogens.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxyl-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are envisaged. Unless otherwise stated, all tautomeric forms of the compounds are envisaged. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are envisaged. Such compounds are useful, for example, as analytical tools, as probes in biological assays, or as therapeutic agents.

### DESCRIPTION OF EXEMPLARY EMBODIMENTS

In one aspect, Compound 1:

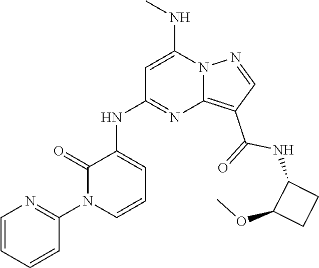 [SMILES: CNc1cc(Nc2cccn(-c3ccccn3)c2=O)nc2c(C(=O)N[C@@H]3CC[C@H]3OC)cnn12]

or a pharmaceutically acceptable salt or solvate thereof, produced by a method disclosed herein, is described.

In one aspect, a compound of Formula I:

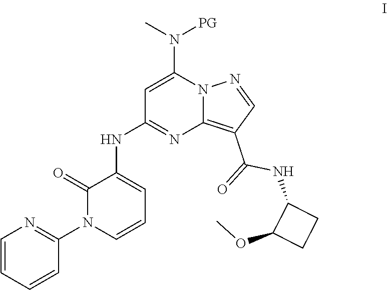

or a pharmaceutically acceptable salt thereof, wherein:
PG is a suitable amino protecting group is described.

In certain embodiments, PG is t-butyloxycarbonyl (BOC), ethyloxycarbonyl, methyloxycarbonyl, trichloroethyloxycarbonyl, allyloxycarbonyl (Alloc), benzyloxocarbonyl (CBZ), allyl, benzyl (Bn), fluorenylmethylcarbonyl (Fmoc), acetyl, chloroacetyl, dichloroacetyl, trichloroacetyl, trifluoroacetyl, phenylacetyl, or benzoyl. In certain embodiments, PG is t-butyloxycarbonyl (BOC).

In one aspect, Compound 2:

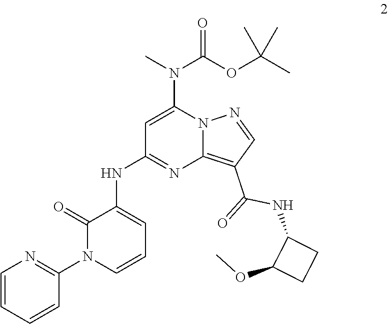 [SMILES: CO[C@@H]1CC[C@H]1NC(=O)c1cnn2c(N(C)C(=O)OC(C)(C)C)cc(Nc3cccn(-c4ccccn4)c3=O)nc12]

or a pharmaceutically acceptable salt thereof is described.

In one aspect, a compound of Formula II:

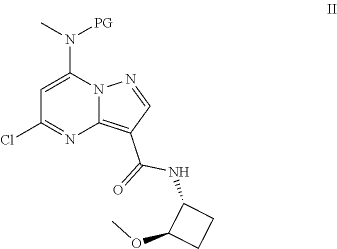

or a pharmaceutically acceptable salt thereof, wherein:
PG is a suitable amino protecting group, is described.

In certain embodiments, PG is t-butyloxycarbonyl (BOC), ethyloxycarbonyl, methyloxycarbonyl, trichloroethyloxycarbonyl, allyloxycarbonyl (Alloc), benzyloxocarbonyl (CBZ), allyl, benzyl (Bn), fluorenylmethylcarbonyl (Fmoc), acetyl, chloroacetyl, dichloroacetyl, trichloroacetyl, trifluoroacetyl, phenylacetyl, or benzoyl. In certain embodiments, PG is t-butyloxycarbonyl (BOC).

In one aspect, Compound 6:

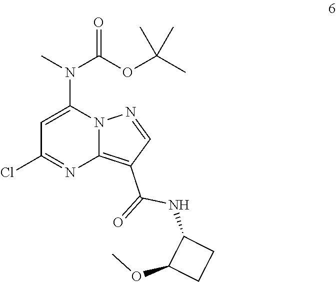 [SMILES: CO[C@@H]1CC[C@H]1NC(=O)c1cnn2c(N(C)C(=O)OC(C)(C)C)cc(Cl)nc12]

or a pharmaceutically acceptable salt thereof is described.

In one aspect, a compound of Formula III:

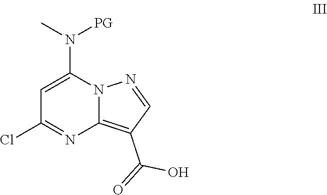

or a pharmaceutically acceptable salt thereof, wherein:
PG is a suitable amino protecting group is described.

Compounds of formula III may be prepared by N-protection of Compound 8:

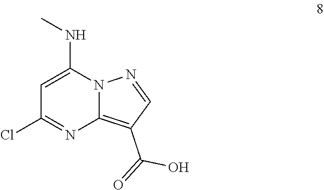

or a pharmaceutically acceptable salt or ester thereof.

In one aspect, Compound 1′:

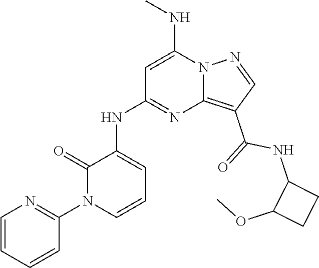 [SMILES: CNc1cc(Nc2cccn(-c3ccccn3)c2=O)nc2c(C(=O)NC3CCC3OC)cnn12]

or a pharmaceutically acceptable salt or solvate thereof, produced by a method disclosed herein is described.

In one aspect, a compound of Formula I′:

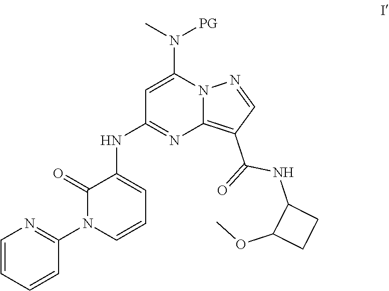

or a pharmaceutically acceptable salt thereof, wherein:
PG is a suitable amino protecting group is described.

In certain embodiments, PG is t-butyloxycarbonyl (BOC), ethyloxycarbonyl, methyloxycarbonyl, trichloroethyloxycarbonyl, allyloxycarbonyl (Alloc), benzyloxocarbonyl (CBZ), allyl, benzyl (Bn), fluorenylmethylcarbonyl (Fmoc), acetyl, chloroacetyl, dichloroacetyl, trichloroacetyl, trifluoroacetyl, phenylacetyl, or benzoyl. In certain embodiments, PG is t-butyloxycarbonyl (BOC).

In one aspect, Compound 2′:

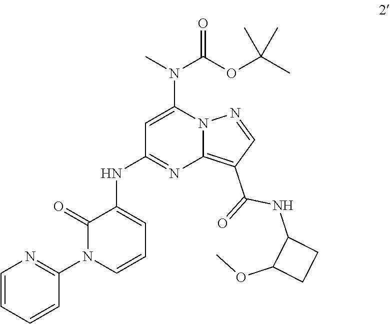 [SMILES: COC1CCC1NC(=O)c1cnn2c(N(C)C(=O)OC(C)(C)C)cc(Nc3cccn(-c4ccccn4)c3=O)nc12]

or a pharmaceutically acceptable salt thereof is described.

In one aspect, a compound of Formula II′:

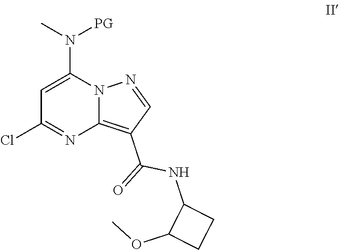

or a pharmaceutically acceptable salt thereof, wherein:
PG is a suitable amino protecting group is described.

In certain embodiments, PG is t-butyloxycarbonyl (BOC), ethyloxycarbonyl, methyloxycarbonyl, trichloroethyloxycarbonyl, allyloxycarbonyl (Alloc), benzyloxocarbonyl (CBZ), allyl, benzyl (Bn), fluorenylmethylcarbonyl (Fmoc), acetyl, chloroacetyl, dichloroacetyl, trichloroacetyl, trifluoroacetyl, phenylacetyl, or benzoyl. In certain embodiments, PG is t-butyloxycarbonyl (BOC).

In one aspect, Compound 6′:

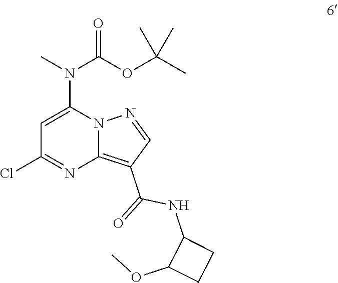 [SMILES: COC1CCC1NC(=O)c1cnn2c(N(C)C(=O)OC(C)(C)C)cc(Cl)nc12]

or a pharmaceutically acceptable salt thereof is described.

In one aspect, a compound of Formula III:

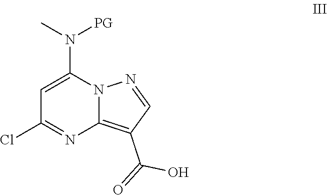

or a pharmaceutically acceptable salt thereof, wherein:
PG is a suitable amino protecting group is described.

Compounds of formula III may be prepared by N-protection of Compound 8:

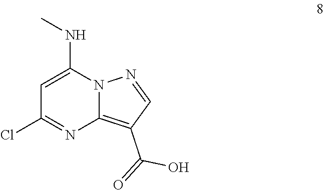

According to embodiments described herein, the deprotection of a protecting group (e.g., PG or PG¹) above, or the addition of a protecting group, includes those protecting groups and methods described in detail in Protecting Groups in Organic Synthesis, T. W. Greene and P. G. M. Wuts, 3^{rd} edition, John Wiley & Sons, 1999, the entirety of each of which is herein incorporated by reference. In some embodiments, the protecting group is a suitable amino protection group.

As used herein, the phrase “suitable amino protecting group” is well known in the art and when taken with the nitrogen to which it is attached, include, but are not limited to, aralkylamines, carbamates, allyl amines, amides, and the like. Examples of mono-protection groups for amines include t-butyloxycarbonyl (BOC), ethyloxycarbonyl, methyloxycarbonyl, trichloroethyloxycarbonyl, allyloxycarbonyl (Alloc), benzyloxocarbonyl (CBZ), allyl, benzyl (Bn), fluorenylmethylcarbonyl (Fmoc), acetyl, chloroacetyl, dichloroacetyl, trichloroacetyl, trifluoroacetyl, phenylacetyl, benzoyl, and the like. Examples of di-protection groups for amines include amines that are substituted with two substituents independently selected from those described above as mono-protection groups, and further include cyclic imides, such as phthalimide, maleimide, succinimide, 2,2,5,5-tetramethyl-1,2,5-azadisilolidine, azide, and the like. It will be appreciated that upon acid hydrolysis of an amino protecting groups, a salt compound thereof is formed. For example, when an amino protecting group is removed by treatment with an acid such as hydrochloric acid, then the resulting amine compound would be formed as its hydrochloride salt. One of ordinary skill in the art would recognize that a wide variety of acids are useful for removing amino protecting groups that are acid-labile and therefore a wide variety of salt forms are contemplated.

In another aspect, a compound selected from one of those in Table 1, or a pharmaceutically acceptable salt thereof, is described.

TABLE 1
Intermediate and target compound(s)

| Compound # | Structure |
|---|---|
| 1 | 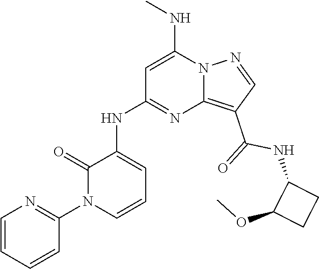 [SMILES: CNc1cc(Nc2cccn(-c3ccccn3)c2=O)nc2c(C(=O)N[C@@H]3CC[C@H]3OC)cnn12] |
| 2 | 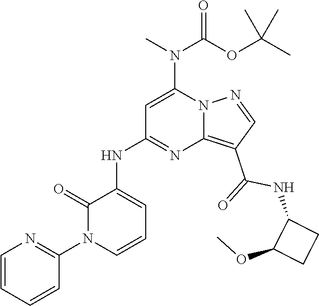 [SMILES: CO[C@@H]1CC[C@H]1NC(=O)c1cnn2c(N(C)C(=O)OC(C)(C)C)cc(Nc3cccn(-c4ccccn4)c3=O)nc12] |
| 3 | 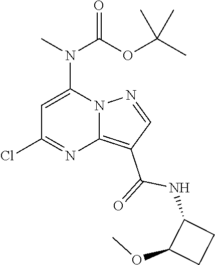 [SMILES: CO[C@@H]1CC[C@H]1NC(=O)c1cnn2c(N(C)C(=O)OC(C)(C)C)cc(Cl)nc12] |

### Exemplary Methods of Synthesis

As described above, methods of synthesizing Compound 1 and compounds of Formula I, I′, II, II′, or III and pharmaceutically acceptable salts thereof are provided herein. In some embodiments, the present compounds are generally prepared according to Scheme 1 set forth below:

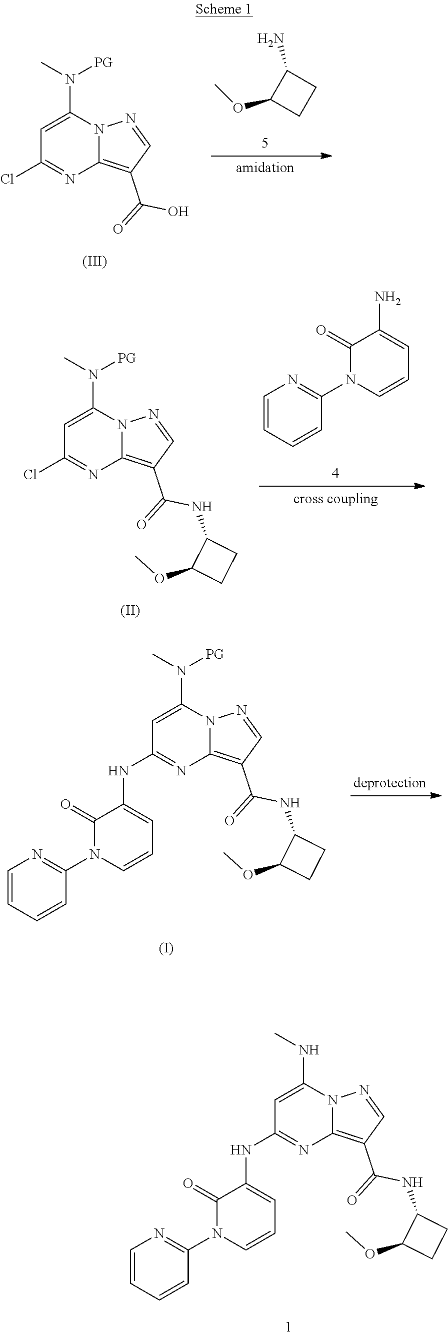

In Scheme 1 above, PG is as defined and described in embodiments herein and includes nitrogen protecting groups well known to this having ordinary skill in the art.

In one aspect, methods for preparing Compound 1:

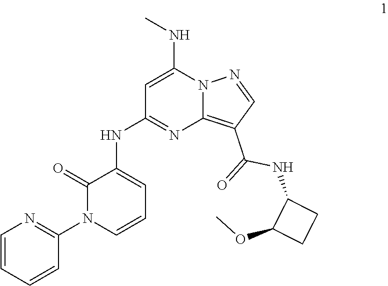 [SMILES: CNc1cc(Nc2cccn(-c3ccccn3)c2=O)nc2c(C(=O)N[C@@H]3CC[C@H]3OC)cnn12]

or a pharmaceutically acceptable salt or solvate thereof, are described, the method including deprotecting a compound of formula I:

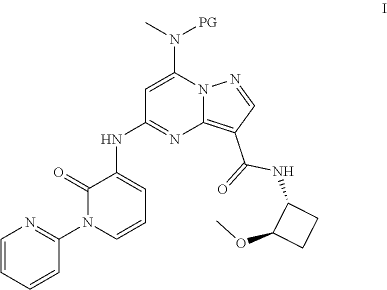

or a pharmaceutically acceptable salt thereof, wherein PG is a suitable amino protecting group.

In certain embodiments, PG is t-butyloxycarbonyl (BOC), ethyloxycarbonyl, methyloxycarbonyl, trichloroethyloxycarbonyl, allyloxycarbonyl (Alloc), benzyloxocarbonyl (CBZ), allyl, benzyl (Bn), fluorenylmethylcarbonyl (Fmoc), acetyl, chloroacetyl, dichloroacetyl, trichloroacetyl, trifluoroacetyl, phenylacetyl, or benzoyl. In certain embodiments, PG is t-butyloxycarbonyl (BOC).

In certain embodiments, the compound of formula I is Compound 2:

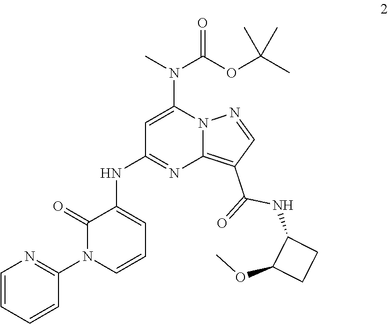 [SMILES: CO[C@@H]1CC[C@H]1NC(=O)c1cnn2c(N(C)C(=O)OC(C)(C)C)cc(Nc3cccn(-c4ccccn4)c3=O)nc12]

or a pharmaceutically acceptable salt thereof.

In some embodiments, the deprotection includes hydrogenolysis, contacting with acid (e.g., HCl), contacting with base (e.g., piperidine, ammonia, K₂CO₃, or methylamine), or heating. According to embodiments described herein, the deprotection of a protecting group (e.g., PG) above includes those protecting groups and methods for their deprotection described in detail in Protecting Groups in Organic Synthesis, T. W. Greene and P. G. M. Wuts, 3^{rd} edition, John Wiley & Sons, 1999, the entirety of which is herein incorporated by reference. In some embodiments, the protecting group is a suitable amino protection group. The deprotection may be performed in any solvent described infra. In some embodiments, the deprotection is performed in an alcohol selected from methanol, ethanol, propanol, butanol, pentanol, or hexanol. In some embodiments, the deprotection is performed in ethanol.

In certain embodiments, the method includes:
a) cross-coupling a compound of formula II:

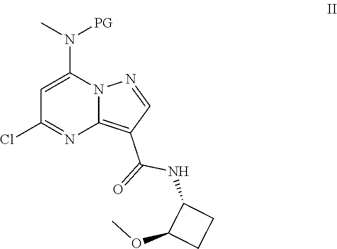

or a pharmaceutically acceptable salt thereof, wherein PG is a suitable amino protecting group; with Compound 4:

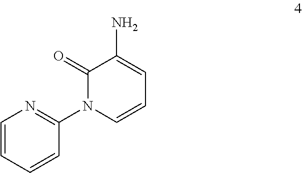 [SMILES: Nc1cccn(-c2ccccn2)c1=O]

to produce a compound of formula I; and
b) deprotecting the compound of formula I:

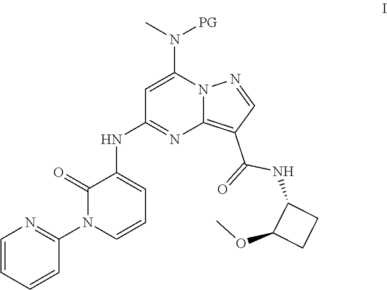

or a pharmaceutically acceptable salt thereof.

In certain embodiments, the compound of formula II is Compound 6:

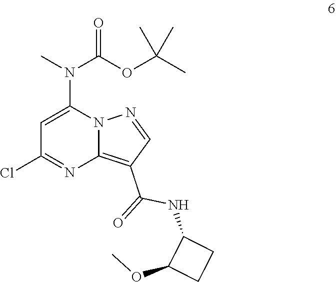 [SMILES: CO[C@@H]1CC[C@H]1NC(=O)c1cnn2c(N(C)C(=O)OC(C)(C)C)cc(Cl)nc12]

or a pharmaceutically acceptable salt thereof.

In one aspect, methods for preparing Compound 1:

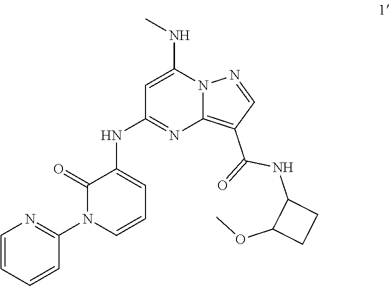 [SMILES: CNc1cc(Nc2cccn(-c3ccccn3)c2=O)nc2c(C(=O)NC3CCC3OC)cnn12]

or a pharmaceutically acceptable salt or solvate thereof, are described, the method including deprotecting a compound of formula I′:

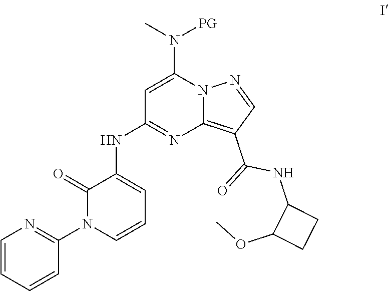

or a pharmaceutically acceptable salt thereof, wherein PG is a suitable amino protecting group.

In certain embodiments, PG is t-butyloxycarbonyl (BOC), ethyloxycarbonyl, methyloxycarbonyl, trichloroethyloxycarbonyl, allyloxycarbonyl (Alloc), benzyloxocarbonyl (CBZ), allyl, benzyl (Bn), fluorenylmethylcarbonyl (Fmoc), acetyl, chloroacetyl, dichloroacetyl, trichloroacetyl, trifluoroacetyl, phenylacetyl, or benzoyl. In certain embodiments, PG is t-butyloxycarbonyl (BOC).

In certain embodiments, the compound of formula I′ is Compound 2′:

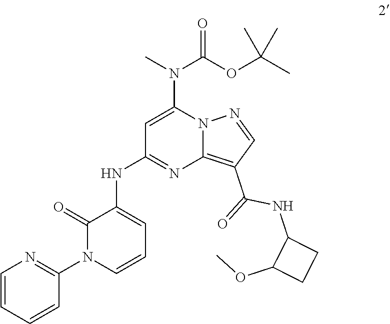 [SMILES: COC1CCC1NC(=O)c1cnn2c(N(C)C(=O)OC(C)(C)C)cc(Nc3cccn(-c4ccccn4)c3=O)nc12]

or a pharmaceutically acceptable salt thereof.

In some embodiments, the deprotection includes hydrogenolysis, contacting with acid (e.g., HCl), contacting with base (e.g., piperidine, ammonia, K₂CO₃, or methylamine), or heating. According to embodiments described herein, the deprotection of a protecting group (e.g., PG) above includes those protecting groups and methods for their deprotection described in detail in Protecting Groups in Organic Synthesis, T. W. Greene and P. G. M. Wuts, 3^{rd} edition, John Wiley & Sons, 1999, the entirety of which is herein incorporated by reference. In some embodiments, the protecting group is a suitable amino protection group. The deprotection may be performed in any solvent described infra. In some embodiments, the deprotection is performed in an alcohol selected from methanol, ethanol, propanol, butanol, pentanol, or hexanol. In some embodiments, the deprotection is performed in ethanol.

In certain embodiments, the method includes:
c) cross-coupling a compound of formula II′:

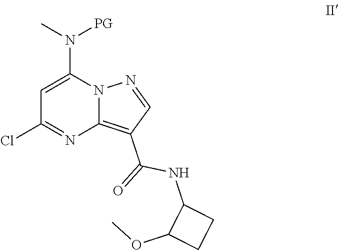

or a pharmaceutically acceptable salt thereof, wherein PG is a suitable amino protecting group; with Compound 4:

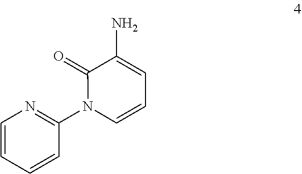 [SMILES: Nc1cccn(-c2ccccn2)c1=O]

to produce a compound of formula I′; and
d) deprotecting the compound of formula I′:

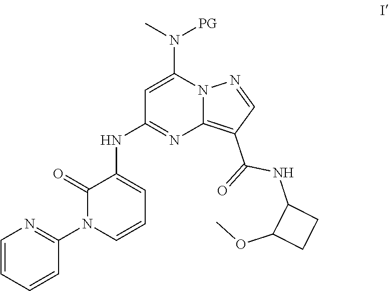

or a pharmaceutically acceptable salt thereof.

In certain embodiments, the compound of formula II′ is Compound 6′:

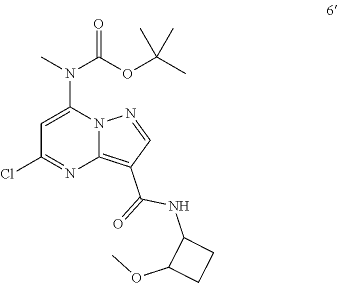 [SMILES: COC1CCC1NC(=O)c1cnn2c(N(C)C(=O)OC(C)(C)C)cc(Cl)nc12]

or a pharmaceutically acceptable salt thereof.

In some embodiments the cross-coupling includes a metal-catalyzed cross-coupling reaction. In certain embodiments, cross-coupling comprises palladium catalyzed cross-coupling (e.g., Buchwald-Hartwig cross-coupling). In some embodiments, the method comprises contacting a palladium compound, a ligand, the compound of formula II or II′ and Compound 4. In some embodiments, the palladium compound comprises Pd(OAc)₂ (palladium (II) acetate), Pd(PPh₃)₄ (tetrakis(triphenylphosphine) palladium(O)), PdCl₂[P(o-Tol)₃]₂ (dichlorobis(tri-o-tolylphosphine)palladium(II)), Pd(dba)₂ (bis(dibenzylideneacetone)palladium(0)), Pd₂(dba)₃ (tris(dibenzylideneacetone) dipalladium(O)), or Pd(dppf)Cl₂ ([1,1′-Bis(diphenylphosphino)ferrocene]dichloropalladium(II)). In some embodiments, the palladium compound comprises Pd(OAc)₂.

In some embodiments, the ligand is Xantphos, diphenylphosphinobinapthyl (BINAP), diphenylphosphinoferrocene (DPPF), tri(o-tolyl)phosphine,

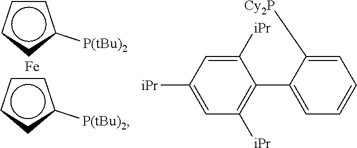

(wherein “Cy” is cyclohexyl), or any palladium cross-coupling ligand known to those of ordinary skill in the art, not limited to those described in Hartwig, J. F. (2008), “Evolution of a Fourth Generation Catalyst for the Amination and Thioetherification of Aryl Halides”, Acc. Chem. Res., 41 (11): 1534-1544, and Surry, D. S.; Buchwald, S. L. (2008), “Biaryl Phosphane Ligands in Palladium-Catalyzed Amination”, Angew. Chem. Int. Ed., 47 (34): 6338-6361, the disclosures of which are hereby incorporated by reference in their entireties. In some embodiments, the ligand is Xantphos. Xantphos has the formula:

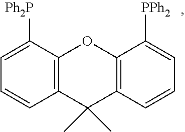

wherein “Ph” is phenyl.

In some embodiments, the method further includes contacting the palladium compound, the ligand, compound 4, and/or the compound of formula II or II′, with a salt. In some embodiments, the salt is NaOt-Bu, LiHMDS, KOH, K₂CO₃, NaOH, Cs₂CO₃, or any other salts described in the publications mentioned supra, Hartwig et al., or Surrey et al. In some embodiments, the salt is K₂CO₃.

In certain embodiments, palladium catalyzed cross-coupling includes contacting Pd(OAc)₂, Xantphos, K₂CO₃, compound 4, and the compound of formula II or II′, in a suitable organic solvent. In some embodiments, the solvent includes DME (1,2-dimethoxyethane). In certain embodiments, the compound of formula I or I′ (cross-coupled product) is washed with N—Ac-L-Cysteine (NAC).

In certain embodiments, the method further includes distilling the NAC washed compound of formula I or I′, dissolved in an organic solvent described infra (i.e., THF) and contacting with activated charcoal. In certain embodiments, the compound of formula I or I′ and activated charcoal are heated. In certain embodiments the heating is to a temperature between about 40° C. to about 50° C., about 50° C. to about 60° C., about 60° C. to about 70° C., or about 70° C. to about 80° C.

In certain embodiments, the compound of formula I or I′ is contacted with activated charcoal. In certain embodiments, the compound of formula I or I′ is contacted with 3-mercaptopropyl ethyl sulphide silica (SPM32). The contacting may be subsequent to the contacting with activated charcoal. In certain embodiments, the compound of formula I or I′ and activated charcoal are heated during contacting. In certain embodiments the heating is to a temperature between about 40° C. to about 50° C., about 50° C. to about 60° C., about 60° C. to about 70° C., or about 70° C. to about 80° C. In certain embodiments, the compound of formula I or I′ is triturated subsequent to SPM32 contacting. The trituration may be from one or more solvents described infra. In certain embodiments, the trituration of the compound of formula I or I′ is from methyl t-butyl ether (MTBE). In certain embodiments the trituration comprises heating to a temperature between about 40° C. to about 50° C., about 50° C. to about 60° C., about 60° C. to about 70° C., or about 70° C. to about 80° C. In certain embodiments, the trituration further comprises cooling the heated compound of formula I or I′, to a temperature between about 0° C. to about 10° C., about 10° C. to about 20° C., or about 20° C. to about 30° C.

In certain embodiments, cross-coupled compounds of formula I or I′ are dissolved in a suitable organic solvent, before and/or after, contacting with charcoal or SPM32; i.e., those solvents described infra. In some embodiments the solvent is DCM (dichloromethane).

Cross-coupling techniques, palladium compounds, ligands and salts, described supra, can be varied as known in the art for example, by using techniques described in Palladium-Catalyzed Coupling Reactions, A. Molnir, 1^{st} edition, Wiley-VCH, 2013, the entirety of each of which is herein incorporated by reference.

In certain embodiments, the method includes:
a) amidating a compound of formula III:

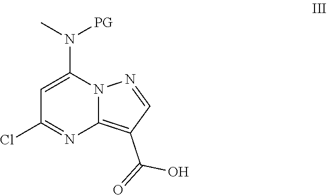

or a pharmaceutically acceptable salt thereof, wherein PG is a suitable amino protecting group; with Compound 5:

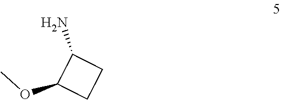

or a pharmaceutically acceptable salt thereof,
to produce a compound of formula II;
b) cross-coupling the compound of formula II:

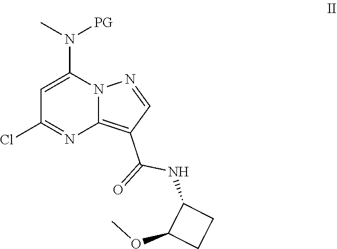

or a pharmaceutically acceptable salt thereof, wherein PG is a suitable amino protecting group; with Compound 4:

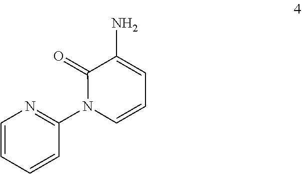 [SMILES: Nc1cccn(-c2ccccn2)c1=O]

or a pharmaceutically acceptable salt thereof, to produce a compound of formula I; and
c) deprotecting the compound of formula I:

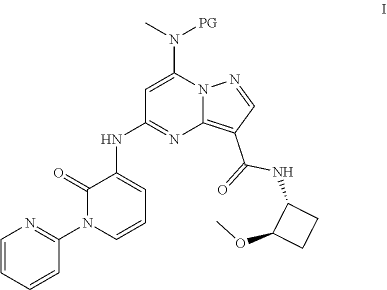

or a pharmaceutically acceptable salt thereof.

In certain embodiments, the compound of formula III is Compound 7:

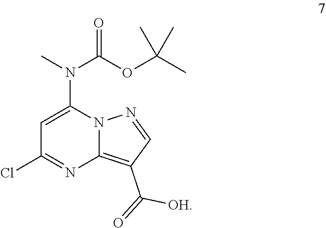 [SMILES: CN(C(=O)OC(C)(C)C)c1cc(Cl)nc2c(C(=O)O)cnn12]

In certain embodiments, the method includes:
d) amidating a compound of formula III:

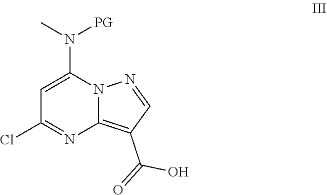

or a pharmaceutically acceptable salt thereof, wherein PG is a suitable amino protecting group; with Compound 5′:

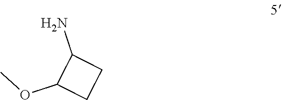

or a pharmaceutically acceptable salt thereof, to produce a compound of formula II′;
e) cross-coupling the compound of formula II′:

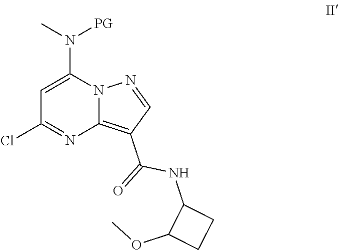

or a pharmaceutically acceptable salt thereof, wherein PG is a suitable amino protecting group; with Compound 4:

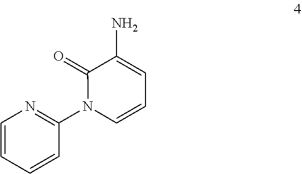 [SMILES: Nc1cccn(-c2ccccn2)c1=O]

or a pharmaceutically acceptable salt thereof, to produce a compound of formula I′; and
f) deprotecting the compound of formula I′:

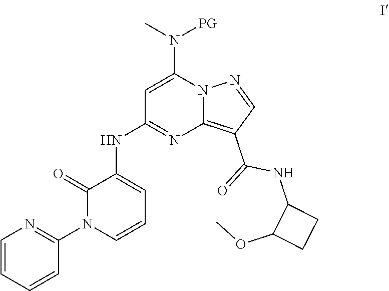

or a pharmaceutically acceptable salt thereof.

In certain embodiments, the compound of formula III is Compound 7:

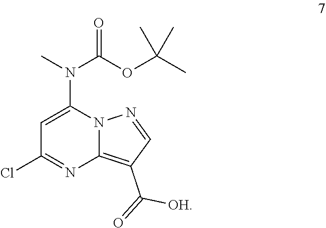 [SMILES: CN(C(=O)OC(C)(C)C)c1cc(Cl)nc2c(C(=O)O)cnn12]

In some embodiments, the compound of formula II or II′ is triturated from an alkane after amidation. The trituration may be before subsequent cross-coupling. The alkane may be selected from butane, pentane, hexane, heptane, octane, nonane, decane, or a mixture thereof. In some embodiments, the compound of formula II or II′ is triturated from heptane after amidation. In some embodiments, the compound of formula II or II′ is triturated from n-heptane after amidation. In some embodiments, the compound of formula TI or TI′ is triturated from n-heptane after amidation, wherein the heptane is at a temperature between about 16° C. to about 18° C., about 18° C. to about 20° C., about 20° C. to about 22° C., or about 22° C. to about 24° C.

Amidation is known to those having ordinary skill in the art to include hydrolysis of a carboxylic acid with an amine to form an amide bond. In certain embodiments, amidation comprises treating the compound of formula III and Compound 5 or 5′ with a base. In some embodiments, the base comprises diisopropylethyl amine (DIPEA), triethylamine (TEA), or pyridine. In some embodiments, the base comprises DIPEA. In some embodiments, the amidation further comprises contacting T3P (propylphosphonic anhydride) with the compound of formula III, Compound 5 or 5′, and the base. In some embodiments, the amidation is neat. In some embodiments the amidation is run in a solvent described infra (i.e., DCM).

In some embodiments, the amidation is conducted at a temperature from about −10° C. to about −5° C., about −5° C. to about 0° C., about 0° C. to about 5° C., about 5° C. to about 10° C., or at about room temperature (22° C.).

### Synthesis of Intermediates

In another aspect, methods for preparing a compound of formula I:

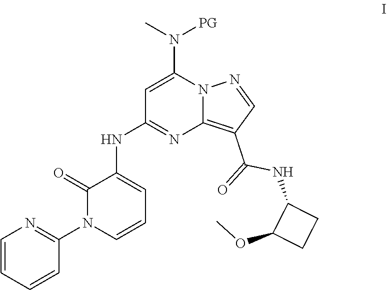

wherein PG is a suitable amino protecting group, are described the method including cross-coupling a compound of formula II:

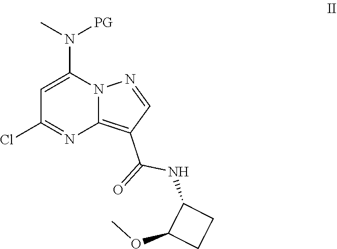

or a pharmaceutically acceptable salt thereof, with Compound 4:

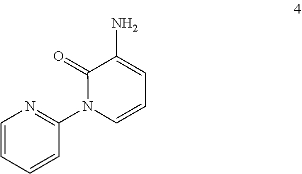 [SMILES: Nc1cccn(-c2ccccn2)c1=O]

or a pharmaceutically acceptable salt thereof.

Cross-coupling may be performed using any of the conditions, reagents, ligands, catalysts, salts, or additives described supra.

In certain embodiments the method includes:
a) amidating a compound of formula III:

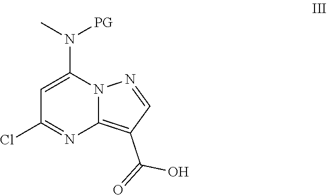

or a pharmaceutically acceptable salt thereof, with Compound 5:

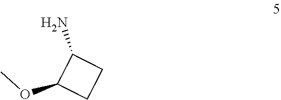

or a pharmaceutically acceptable salt thereof,
to produce the compound of formula II; and
b) cross-coupling the compound of formula II:

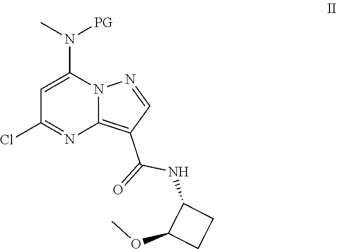

or a pharmaceutically acceptable salt thereof, with Compound 4:

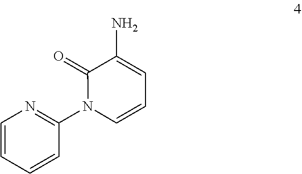 [SMILES: Nc1cccn(-c2ccccn2)c1=O]

or a pharmaceutically acceptable salt thereof,
to produce the compound of formula I.

Amidation and/or cross-coupling may be performed using any of the conditions, reagents, or additives described supra.

In another aspect, methods for preparing a compound of formula II:

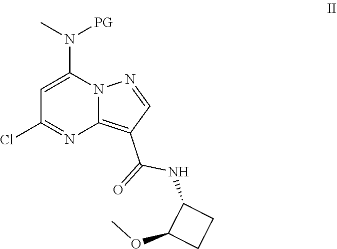

or a pharmaceutically acceptable salt thereof are described; the method including amidating a compound of formula III:

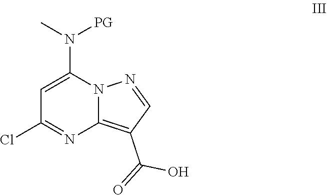

or a pharmaceutically acceptable salt thereof, with Compound 5:

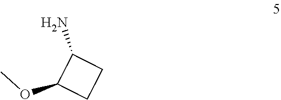

or a pharmaceutically acceptable salt thereof, to produce the compound of formula II.

In another aspect, methods for preparing a compound of formula I′:

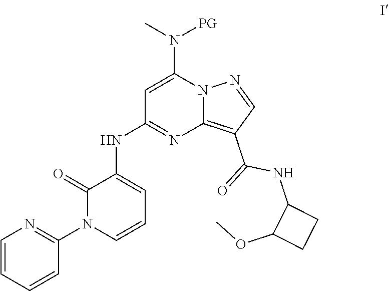

wherein PG is a suitable amino protecting group are described, the method including cross-coupling a compound of formula II′:

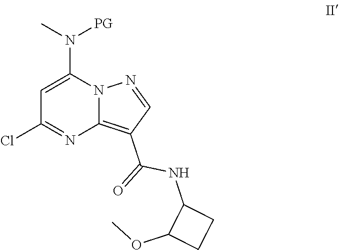

or a pharmaceutically acceptable salt thereof, with Compound 4:

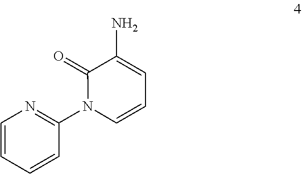 [SMILES: Nc1cccn(-c2ccccn2)c1=O]

or a pharmaceutically acceptable salt thereof.

Cross-coupling may be performed using any of the conditions, reagents, ligands, catalysts, salts, or additives described supra.

In certain embodiments the method includes:
c) amidating a compound of formula III:

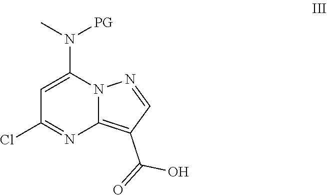

or a pharmaceutically acceptable salt thereof, with Compound 5′:

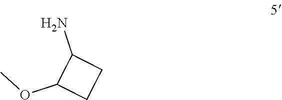

or a pharmaceutically acceptable salt thereof,
to produce the compound of formula II′; and
d) cross-coupling the compound of formula II′:

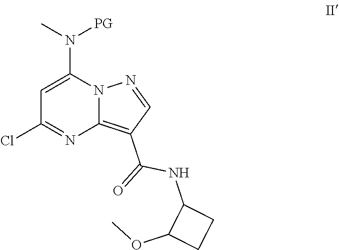

or a pharmaceutically acceptable salt thereof, with Compound 4:

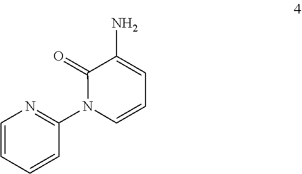 [SMILES: Nc1cccn(-c2ccccn2)c1=O]

or a pharmaceutically acceptable salt thereof,
to produce the compound of formula I′.

Amidation and/or cross-coupling may be performed using any of the conditions, reagents, or additives described supra.

In another aspect, methods for preparing a compound of formula II′:

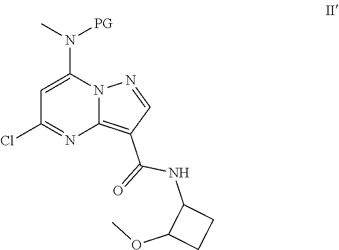

or a pharmaceutically acceptable salt thereof are described; the method including amidating a compound of formula III:

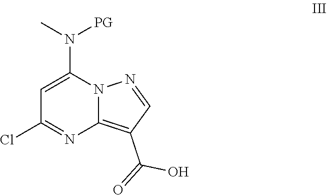

or a pharmaceutically acceptable salt thereof, with Compound 5′:

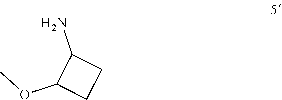

or a pharmaceutically acceptable salt thereof, to produce the compound of formula II′.

Amidation may be performed using any of the conditions, reagents, or additives described supra.

In some embodiments, the compound of formula II or II′ is triturated from heptane after amidation. In some embodiments, the compound of formula II or II′ is triturated from n-heptane after amidation. In some embodiments, the compound of formula II or II′ is triturated from n-heptane after amidation, wherein the heptane is at a temperature between about 16° C. to about 18° C., about 18° C. to about 20° C., about 20° C. to about 22° C., or about 22° C. to about 24° C.

In certain embodiments, amidation includes treating the compound of formula III and Compound 5 or 5′ with a base. In some embodiments, the base comprises diisopropylethyl amine (DIPEA), triethylamine (TEA), or pyridine. In some embodiments, the base comprises DIPEA. Amidation and cross-coupling may be performed using any of the conditions, reagents, or additives described supra.

In any of the aforementioned methods of preparing, the reactions may be run neat or in a solvent. A suitable medium is a solvent or a solvent mixture that, in combination with the combined compounds, may facilitate the progress of the reaction therebetween. The suitable solvent may solubilize one or more of the reaction components, or, alternatively, the suitable solvent may facilitate the agitation of a suspension of one or more of the reaction components. Examples of suitable solvents are a protic solvent, a halogenated hydrocarbon, an ether, an ester, an aromatic hydrocarbon, a polar or a non-polar aprotic solvent, or any mixtures thereof. Such mixtures include, for example, mixtures of protic and non-protic solvents such as benzene/methanol/water; benzene/water; DME/water, and the like.

These and other such suitable solvents may be interchanged are well known in the art, e.g., see, “Advanced Organic Chemistry”, Jerry March, 5^{th} edition, John Wiley and Sons, N.Y.

### Uses, Formulation and Administration

### Pharmaceutically Acceptable Compositions

According to another embodiment, provided is a composition comprising a compound produced by the method herein and a pharmaceutically acceptable carrier, adjuvant, or vehicle. The amount of compound in compositions is such that is effective to measurably inhibit a TYK2 protein kinase, or a mutant thereof, in a biological sample or in a patient. In certain embodiments, the amount of compound in compositions is such that is effective to measurably inhibit a TYK2 protein kinase, or a mutant thereof, in a biological sample or in a patient. In certain embodiments, a composition is formulated for administration to a patient in need of such composition. In some embodiments, a composition is formulated for oral administration to a patient.

The term “patient,” as used herein, means an animal, preferably a mammal, and most preferably a human.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound (Compound 1) that, upon administration to a recipient, is capable of providing, either directly or indirectly, a compound or an inhibitorily active metabolite or residue thereof.

As used herein, the term “inhibitorily active metabolite or residue thereof” means that a metabolite or residue thereof is also an inhibitor of a TYK2 protein kinase, or a mutant thereof.

Compositions may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, pharmaceutically acceptable compositions may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of Compound 1 include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Most preferably, pharmaceutically acceptable compositions are formulated for oral administration. Such formulations may be administered with or without food. In some embodiments, pharmaceutically acceptable compositions are administered without food. In other embodiments, pharmaceutically acceptable compositions are administered with food.

The amount of compound that may be combined with the carrier materials to produce a composition in a single dosage form will vary depending upon the host treated, the particular mode of administration. Preferably, provided compositions should be formulated so that a dosage of between 0.01-100 mg/kg body weight/day of the inhibitor can be administered to a patient receiving these compositions.

It should also be understood that a specific dosage and treatment regimen for any particular patient will depend upon a variety of factors, including the activity of Compound 1 employed, the age, body weight, general health, sex, diet, time of administration, rate of excretion, drug combination, and the judgment of the treating physician and the severity of the particular disease being treated.

### Uses of Compounds and Pharmaceutically Acceptable Compositions

Compound 1 and compositions described herein are generally useful for the inhibition of kinase activity of one or more enzymes. In some embodiments the kinase inhibited by Compound 1 and methods herein is TYK2.

TYK2 is a non-receptor tyrosine kinase member of the Janus kinase (JAKs) family of protein kinases. The mammalian JAK family consists of four members, TYK2, JAK1, JAK2, and JAK3. JAK proteins, including TYK2, are integral to cytokine signaling. TYK2 associates with the cytoplasmic domain of type I and type II cytokine receptors, as well as interferon types I and III receptors, and is activated by those receptors upon cytokine binding. Cytokines implicated in TYK2 activation include interferons (e.g. IFN-α, IFN-β, IFN-κ, IFN-δ, IFN—ε, IFN-τ, IFN-ω, and IFN-ζ (also known as limitin), and interleukins (e.g. IL-4, IL-6, IL-10, IL-11, IL-12, IL-13, IL-22, IL-23, IL-27, IL-31, oncostatin M, ciliary neurotrophic factor, cardiotrophin 1, cardiotrophin-like cytokine, and LIF). Velasquez et al., “A protein kinase in the interferon α/β signaling pathway,” Cell (1992) 70:313; Stahl et al., “Association and activation of Jak-Tyk kinases by CNTF-LIF-OSM-IL-6$ receptor components,” Science (1994) 263:92; Finbloom et al., “IL-10 induces the tyrosine phosphorylation of Tyk2 and Jak1 and the differential assembly of Stat1 and Stat3 complexes in human T cells and monocytes,” J. Immunol. (1995) 155:1079; Bacon et al., “Interleukin 12 (IL-12) induces tyrosine phosphorylation of Jak2 and Tyk2: differential use of Janus family kinases by IL-2 and IL-12,” J. Exp. Med. (1995) 181:399; Welham et al., “Interleukin-13 signal transduction in lymphohemopoietic cells: similarities and differences in signal transduction with interleukin-4 and insulin,” J. Biol. Chem. (1995) 270:12286; Parham et al., “A receptor for the heterodimeric cytokine IL-23 is composed of IL-12RP1 and a novel cytokine receptor subunit, IL-23R,” J. Immunol. (2002) 168:5699. The activated TYK2 then goes on to phosphorylate further signaling proteins such as members of the STAT family, including STAT1, STAT2, STAT4, and STAT6.

TYK2 activation by IL-23, has been linked to inflammatory bowel disease (IBD), Crohn's disease, and ulcerative colitis. Duerr et al., “A Genome-Wide Association Study Identifies IL23R as an Inflammatory Bowel Disease Gene,” Science (2006) 314:1461-1463. As the downstream effector of IL-23, TYK2 also plays a role in psoriasis, ankylosing spondylitis, and Behget's disease. Cho et al., “Genomics and the multifactorial nature of human autoimmune disease,” N. Engl. J. Med (2011) 365:1612-1623; Cortes et al., “Identification of multiple risk variants for ankylosing spondylitis through high-density genotyping of immune-related loci,” Nat. Genet. (2013) 45(7):730-738; Remmers et al., “Genome-wide association study identifies variants in the MHC class I, IL10, and IL23R-IL12RB2 regions associated with Behget's disease,” Nat. Genet. (2010) 42:698-702. A genome-wide association study of 2,622 individuals with psoriasis identified associations between disease susceptibility and TYK2. Strange et al., “A genome-wide association study identifies new psoriasis susceptibility loci and an interaction between HLA-C and ERAP1,” Nat. Genet. (2010) 42:985-992. Knockout or tyrphostin inhibition of TYK2 significantly reduces both IL-23 and IL-22-induced dermatitis. Ishizaki et al., “Tyk2 is a therapeutic target for psoriasis-like skin inflammation,” Intl. Immunol. (2013), doi: 10.1093/intimm/dxt062.

TYK2 also plays a role in respiratory diseases such as asthma, chronic obstructive pulmonary disease (COPD), lung cancer, and cystic fibrosis. Goblet cell hyperplasia (GCH) and mucous hypersecretion is mediated by IL-13-induced activation of TYK2, which in turn activates STAT6. Zhang et al., “Docking protein Gab2 regulates mucin expression and goblet cell hyperplasia through TYK2/STAT6 pathway,” FASEB J. (2012) 26:1-11. Decreased TYK2 activity leads to protection of joints from collagen antibody-induced arthritis, a model of human rheumatoid arthritis. Mechanistically, decreased Tyk2 activity reduced the production of Tₕ1/Tₕ17-related cytokines and matrix metalloproteases, and other key markers of inflammation. Ishizaki et al., “Tyk2 deficiency protects joints against destruction in anti-type II collagen antibody-induced arthritis in mice,” Intl. Immunol. (2011) 23(9):575-582.

TYK2 knockout mice showed complete resistance in experimental autoimmune encephalomyelitis (EAE, an animal model of multiple sclerosis (MS)), with no infiltration of CD4 T cells in the spinal cord, as compared to controls, suggesting that TYK2 is essential to pathogenic CD4-mediated disease development in MS. Oyamada et al., “Tyrosine Kinase 2 Plays Critical Roles in the Pathogenic CD4 T Cell Responses for the Development of Experimental Autoimmune Encephalomyelitis,” J. Immunol. (2009) 183:7539-7546. This corroborates earlier studies linking increased TYK2 expression with MS susceptibility. Ban et al., “Replication analysis identifies TYK2 as a multiple sclerosis susceptibility factor,” Eur J. Hum. Genet. (2009) 17:1309-1313. Loss of function mutation in TYK2, leads to decreased demyelination and increased remyelination of neurons, further suggesting a role for TYK2 inhibitors in the treatment of MS and other CNS demyelination disorders.

TYK2 is the sole signaling messenger common to both IL-12 and IL-23. TYK2 knockout reduced methylated BSA injection-induced footpad thickness, imiquimod-induced psoriasis-like skin inflammation, and dextran sulfate sodium or 2,4,6-trinitrobenzene sulfonic acid-induced colitis in mice.

Joint linkage and association studies of various type I IFN signaling genes with systemic lupus erythematosus (SLE, an autoimmune disorder), showed a strong, and significant correlation between loss of function mutations to TYK2 and decreased prevalence of SLE in families with affected members. Sigurdsson et al., “Polymorphisms in the Tyrosine Kinase 2 and Interferon Regulatory Factor 5 Genes Are Associated with Systemic Lupis Erythematosus,” Am. J. Hum. Genet. (2005) 76:528-537. Genome-wide association studies of individuals with SLE versus an unaffected cohort showed highly significant correlation between the TYK2 locus and SLE. Graham et al., “Association ofNCF2, IKZFI, IRF8, IFIHI, and TYK2 with Systemic Lupus Erythematosus,” PLoS Genetics (2011) 7(10):e1002341.

TYK2 has been shown to play an important role in maintaining tumor surveillance and TYK2 knockout mice showed compromised cytotoxic T cell response, and accelerated tumor development. However, these effects were linked to the efficient suppression of natural killer (NK) and cytotoxic T lymphocytes, suggesting that TYK2 inhibitors would be highly suitable for the treatment of autoimmune disorders or transplant rejection. Although other JAK family members such as JAK3 have similar roles in the immune system, TYK2 has been suggested as a superior target because of its involvement in fewer and more closely related signaling pathways, leading to fewer off-target effects. Simma et al. “Identification of an Indispensable Role for Tyrosine Kinase 2 in CTL-Mediated Tumor Surveillance,” Cancer Res. (2009) 69:203-211.

However, paradoxically to the decreased tumor surveillance observed by Simma et al., studies in T-cell acute lymphoblastic leukemia (T-ALL) indicate that T-ALL is highly dependent on IL-10 via TYK2 via STAT1-mediated signal transduction to maintain cancer cell survival through upregulation of anti-apoptotic protein BCL2. Knockdown of TYK2, but not other JAK family members, reduced cell growth. Specific activating mutations to TYK2 that promote cancer cell survival include those to the FERM domain (G36D, S47N, and R425H), the JH2 domain (V7311), and the kinase domain (E957D and R1027H). However, it was also identified that the kinase function of TYK2 is required for increased cancer cell survival, as TYK2 enzymes featuring kinase-dead mutations (M978Y or M978F) in addition to an activating mutation (E957D) resulted in failure to transform. Sanda et al. “TYK2-STAT1-BCL2 Pathway Dependence in T-Cell Acute Lymphoblastic Leukemia,” Cancer Disc. (2013) 3(5):564-577.

Thus, selective inhibition of TYK2 has been suggested as a suitable target for patients with IL-10 and/or BCL2-addicted tumors, such as 70% of adult T-cell leukemia cases. Fontan et al. “Discovering What Makes STAT Signaling TYK in T-ALL,” Cancer Disc. (2013) 3:494-496.

TYK2 mediated STAT3 signaling has also been shown to mediate neuronal cell death caused by amyloid-β (Aβ) peptide. Decreased TYK2 phosphorylation of STAT3 following Aρ administration lead to decreased neuronal cell death, and increased phosphorylation of STAT3 has been observed in postmortem brains of Alzheimer's patients. Wan et al. “Tyk/STAT3 Signaling Mediates β-Amyloid-Induced Neuronal Cell Death: Implications in Alzheimer's Disease,” J. Neurosci. (2010) 30(20):6873-6881.

Inhibition of JAK-STAT signaling pathways is also implicated in hair growth, and the reversal of the hair loss associated with alopecia areata. Xing et al., “Alopecia areata is driven by cytotoxic T lymphocytes and is reversed by JAK inhibition,” Nat. Med. (2014) 20: 1043-1049; Harel et al., “Pharmacologic inhibition of JAK-STAT signaling promotes hair growth,” Sci. Adv. (2015) 1(9):e1500973.

Accordingly, compounds that inhibit the activity of TYK2 are beneficial, especially those with selectivity over JAK2. Such compounds should deliver a pharmacological response that favorably treats one or more of the conditions described herein without the side-effects associated with the inhibition of JAK2.

Even though TYK2 inhibitors are known in the art, there is a continuing need to provide novel inhibitors having more effective or advantageous pharmaceutically relevant properties. For example, compounds with increased activity, selectivity over other JAK kinases (especially JAK2), and ADMET (absorption, distribution, metabolism, excretion, and/or toxicity) properties. Thus, in some embodiments, a Compound 1 inhibitor of TYK2 shows selectivity over JAK2.

The activity of Compound 1 utilized herein as an inhibitor of TYK2, or a mutant thereof, may be assayed in vitro, in vivo or in a cell line. In vitro assays include assays that determine inhibition of either the phosphorylation activity and/or the subsequent functional consequences, or ATPase activity of activated TYK2, or a mutant thereof. Alternate in vitro assays quantitate the ability of the inhibitor to bind to TYK2. Inhibitor binding may be measured by radiolabeling the inhibitor prior to binding, isolating the inhibitor/TYK2 complex and determining the amount of radiolabel bound. Alternatively, inhibitor binding may be determined by running a competition experiment where new inhibitors are incubated with TYK2 bound to known radioligands. Representative in vitro and in vivo assays useful in assaying a TYK2 inhibitor include those described and disclosed in, e.g., each of which is herein incorporated by reference in its entirety.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

Compound 1 is an inhibitor of TYK2 and is therefore useful for treating one or more disorders associated with activity of TYK2 or mutants thereof. Thus, in certain embodiments, a method for treating a TYK2-mediated disorder comprising the step of administering to a patient in need thereof Compound 1 produced by the method described herein, or pharmaceutically acceptable composition thereof.

As used herein, the term “TYK2-mediated” disorders, diseases, and/or conditions as used herein means any disease or other deleterious condition in which TYK2 or a mutant thereof is known to play a role. Accordingly, another embodiment relates to treating or lessening the severity of one or more diseases in which TYK2, or a mutant thereof, is known to play a role. Such TYK2-mediated disorders include but are not limited to autoimmune disorders, inflammatory disorders, proliferative disorders, endocrine disorders, neurological disorders and disorders associated with transplantation.

In some embodiments a method for treating one or more disorders, wherein the disorders are selected from autoimmune disorders, inflammatory disorders, proliferative disorders, endocrine disorders, neurological disorders, and disorders associated with transplantation, said method comprising administering to a patient in need thereof, a pharmaceutical composition comprising an effective amount of Compound 1 produced by the method herein, or a pharmaceutically acceptable salt thereof.

In some embodiments, the disorder is an autoimmune disorder. In some embodiments the disorder is selected from type 1 diabetes, cutaneous lupus erythematosus, systemic lupus erythematosus, multiple sclerosis, psoriasis, Behget's disease, POEMS syndrome, Crohn's disease, ulcerative colitis, and inflammatory bowel disease.

In some embodiments, the disorder is an inflammatory disorder. In some embodiments, the inflammatory disorder is rheumatoid arthritis, asthma, chronic obstructive pulmonary disease, psoriasis, hepatomegaly, Crohn's disease, ulcerative colitis, inflammatory bowel disease.

In some embodiments, the disorder is a proliferative disorder. In some embodiments, the proliferative disorder is a hematological cancer. In some embodiments the proliferative disorder is a leukemia. In some embodiments, the leukemia is a T-cell leukemia. In some embodiments the T-cell leukemia is T-cell acute lymphoblastic leukemia (T-ALL). In some embodiments the proliferative disorder is polycythemia vera, myelofibrosis, essential or thrombocytosis.

In some embodiments, the disorder is an endocrine disorder. In some embodiments, the endocrine disorder is polycystic ovary syndrome, Crouzon's syndrome, or type 1 diabetes.

In some embodiments, the disorder is a neurological disorder. In some embodiments, the neurological disorder is Alzheimer's disease.

In some embodiments the proliferative disorder is associated with one or more activating mutations in TYK2. In some embodiments, the activating mutation in TYK2 is a mutation to the FERM domain, the JH2 domain, or the kinase domain. In some embodiments the activating mutation in TYK2 is selected from G36D, S47N, R425H, V731I, E957D, and R1027H.

In some embodiments, the disorder is associated with transplantation. In some embodiments the disorder associated with transplantation is transplant rejection, or graft versus host disease.

In some embodiments the disorder is associated with type I interferon, IL-10, IL-12, or IL-23 signaling. In some embodiments the disorder is associated with type I interferon signaling. In some embodiments the disorder is associated with IL-10 signaling. In some embodiments the disorder is associated with IL-12 signaling. In some embodiments the disorder is associated with IL-23 signaling.

Compound 1 produced by the method herein is also useful in the treatment of inflammatory or allergic conditions of the skin, for example psoriasis, contact dermatitis, atopic dermatitis, alopecia areata, erythema multiforma, dermatitis herpetiformis, scleroderma, vitiligo, hypersensitivity angiitis, urticaria, bullous pemphigoid, lupus erythematosus, cutaneous lupus erythematosus, systemic lupus erythematosus, pemphigus vulgaris, pemphigus foliaceus, paraneoplastic pemphigus, epidermolysis bullosa acquisita, acne vulgaris, and other inflammatory or allergic conditions of the skin.

Compound 1 produced by the method herein may also be used for the treatment of other diseases or conditions, such as diseases or conditions having an inflammatory component, for example, treatment of diseases and conditions of the eye such as ocular allergy, conjunctivitis, keratoconjunctivitis sicca, and vernal conjunctivitis, diseases affecting the nose including allergic rhinitis, and inflammatory disease in which autoimmune reactions are implicated or having an autoimmune component or etiology, including autoimmune hematological disorders (e.g. hemolytic anemia, aplastic anemia, pure red cell anemia and idiopathic thrombocytopenia), cutaneous lupus erythematosus, systemic lupus erythematosus, rheumatoid arthritis, polychondritis, scleroderma, Wegener granulomatosis, dermatomyositis, chronic active hepatitis, myasthenia gravis, Steven-Johnson syndrome, idiopathic sprue, autoimmune inflammatory bowel disease (e.g. ulcerative colitis and Crohn's disease), irritable bowel syndrome, celiac disease, periodontitis, hyaline membrane disease, kidney disease, glomerular disease, alcoholic liver disease, multiple sclerosis, endocrine ophthalmopathy, Grave's disease, sarcoidosis, alveolitis, chronic hypersensitivity pneumonitis, multiple sclerosis, primary biliary cirrhosis, uveitis (anterior and posterior), Sjogren's syndrome, keratoconjunctivitis sicca and vernal keratoconjunctivitis, interstitial lung fibrosis, psoriatic arthritis, systemic juvenile idiopathic arthritis, cryopyrin-associated periodic syndrome, nephritis, vasculitis, diverticulitis, interstitial cystitis, glomerulonephritis (with and without nephrotic syndrome, e.g. including idiopathic nephrotic syndrome or minimal change nephropathy), chronic granulomatous disease, endometriosis, leptospiriosis renal disease, glaucoma, retinal disease, ageing, headache, pain, complex regional pain syndrome, cardiac hypertrophy, muscle wasting, catabolic disorders, obesity, fetal growth retardation, hypercholesterolemia, heart disease, chronic heart failure, mesothelioma, anhidrotic ectodermal dysplasia, Behcet's disease, incontinentia pigmenti, Paget's disease, pancreatitis, hereditary periodic fever syndrome, asthma (allergic and non-allergic, mild, moderate, severe, bronchitic, and exercise-induced), acute lung injury, acute respiratory distress syndrome, eosinophilia, hypersensitivities, anaphylaxis, nasal sinusitis, ocular allergy, silica induced diseases, COPD (reduction of damage, airways inflammation, bronchial hyperreactivity, remodeling or disease progression), pulmonary disease, cystic fibrosis, acid-induced lung injury, pulmonary hypertension, polyneuropathy, cataracts, muscle inflammation in conjunction with systemic sclerosis, inclusion body myositis, myasthenia gravis, thyroiditis, Addison's disease, lichen planus, Type 1 diabetes, or Type 2 diabetes, appendicitis, atopic dermatitis, asthma, allergy, blepharitis, bronchiolitis, bronchitis, bursitis, cervicitis, cholangitis, cholecystitis, chronic graft rejection, colitis, conjunctivitis, Crohn's disease, cystitis, dacryoadenitis, dermatitis, dermatomyositis, encephalitis, endocarditis, endometritis, enteritis, enterocolitis, epicondylitis, epididymitis, fasciitis, fibrositis, gastritis, gastroenteritis, Henoch-Schonlein purpura, hepatitis, hidradenitis suppurativa, immunoglobulin A nephropathy, interstitial lung disease, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, polymyositis, proctitis, prostatitis, pyelonephritis, rhinitis, salpingitis, sinusitis, stomatitis, synovitis, tendonitis, tonsillitis, ulcerative colitis, uveitis, vaginitis, vasculitis, or vulvitis.

In some embodiments the inflammatory disease which can be treated according to the methods is selected from acute and chronic gout, chronic gouty arthritis, psoriasis, psoriatic arthritis, rheumatoid arthritis, Juvenile rheumatoid arthritis, Systemic juvenile idiopathic arthritis (SJIA), Cryopyrin Associated Periodic Syndrome (CAPS), and osteoarthritis.

In some embodiments the inflammatory disease which can be treated according to the methods is a Tₕ1 or Tₕ17 mediated disease. In some embodiments the Th17 mediated disease is selected from cutaneous lupus erythematosus, Systemic lupus erythematosus, Multiple sclerosis, and inflammatory bowel disease (including Crohn's disease or ulcerative colitis).

In some embodiments the inflammatory disease which can be treated according to the methods is selected from Sjogren's syndrome, allergic disorders, osteoarthritis, conditions of the eye such as ocular allergy, conjunctivitis, keratoconjunctivitis sicca and vernal conjunctivitis, and diseases affecting the nose such as allergic rhinitis.

Furthermore, described herein is the use of a Compound 1 according to the definitions herein, or a pharmaceutically acceptable salt, or a hydrate or solvate thereof for the preparation of a medicament for the treatment of an autoimmune disorder, an inflammatory disorder, or a proliferative disorder, or a disorder commonly occurring in connection with transplantation.

### Combination Therapies

Depending upon the particular condition, or disease, to be treated, additional therapeutic agents, which are normally administered to treat that condition, may be administered in combination with Compound 1 produced by the methods herein and compositions. As used herein, additional therapeutic agents that are normally administered to treat a particular disease, or condition, are known as “appropriate for the disease, or condition, being treated.”

In certain embodiments, a provided combination, or composition thereof, is administered in combination with another therapeutic agent.

Examples of agents the combinations may also be combined with include, without limitation: treatments for Alzheimer's Disease such as Aricept® and Excelon®; treatments for HIV such as ritonavir; treatments for Parkinson's Disease such as L-DOPA/carbidopa, entacapone, ropinirole, pramipexole, bromocriptine, pergolide, trihexephendyl, and amantadine; agents for treating Multiple Sclerosis (MS) such as beta interferon (e.g., Avonex© and Rebif®), Copaxone®, and mitoxantrone; treatments for asthma such as albuterol and Singulair®; agents for treating schizophrenia such as zyprexa, risperdal, seroquel, and haloperidol; anti-inflammatory agents such as corticosteroids, TNF blockers, IL-1 RA, azathioprine, cyclophosphamide, and sulfasalazine; immunomodulatory and immunosuppressive agents such as cyclosporin, tacrolimus, rapamycin, mycophenolate mofetil, interferons, corticosteroids, cyclophosphamide, azathioprine, and sulfasalazine; neurotrophic factors such as acetylcholinesterase inhibitors, MAO inhibitors, interferons, anti-convulsants, ion channel blockers, riluzole, and anti-Parkinsonian agents; agents for treating cardiovascular disease such as beta-blockers, ACE inhibitors, diuretics, nitrates, calcium channel blockers, and statins; agents for treating liver disease such as corticosteroids, cholestyramine, interferons, and anti-viral agents; agents for treating blood disorders such as corticosteroids, anti-leukemic agents, and growth factors; agents that prolong or improve pharmacokinetics such as cytochrome P450 inhibitors (i.e., inhibitors of metabolic breakdown) and CYP3A4 inhibitors (e.g., ketokenozole and ritonavir), and agents for treating immunodeficiency disorders such as gamma globulin.

In certain embodiments, combination therapies, or a pharmaceutically acceptable composition thereof, are administered in combination with a monoclonal antibody or an siRNA therapeutic.

Those additional agents may be administered separately from a provided combination therapy, as part of a multiple dosage regimen. Alternatively, those agents may be part of a single dosage form, mixed together with a compound in a single composition. If administered as part of a multiple dosage regime, the two active agents may be submitted simultaneously, sequentially or within a period of time from one another normally within five hours from one another.

As used herein, the term “combination,” “combined,” and related terms refers to the simultaneous or sequential administration of therapeutic agents. For example, a combination may be administered with another therapeutic agent simultaneously or sequentially in separate unit dosage forms or together in a single unit dosage form.

The amount of additional therapeutic agent present in the compositions will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

In one embodiment, a composition comprising Compound 1 and one or more additional therapeutic agents is envisaged. The therapeutic agent may be administered together with Compound 1, or may be administered prior to or following administration of Compound 1. Suitable therapeutic agents are described in further detail below. In certain embodiments, Compound 1 may be administered up to 5 minutes, 10 minutes, 15 minutes, 30 minutes, 1 hour, 2 hours, 3 hours, 4 hours, 5, hours, 6 hours, 7 hours, 8 hours, 9 hours, 10 hours, 11 hours, 12 hours, 13 hours, 14 hours, 15 hours, 16 hours, 17 hours, or 18 hours before the therapeutic agent. In other embodiments, Compound 1 may be administered up to 5 minutes, 10 minutes, 15 minutes, 30 minutes, 1 hour, 2 hours, 3 hours, 4 hours, 5, hours, 6 hours, 7 hours, 8 hours, 9 hours, 10 hours, 11 hours, 12 hours, 13 hours, 14 hours, 15 hours, 16 hours, 17 hours, or 18 hours following the therapeutic agent.

In another embodiment, a method of treating an inflammatory disease, disorder or condition by administering to a patient in need thereof Compound 1 and one or more additional therapeutic agents is described. Such additional therapeutic agents may be small molecules or recombinant biologic agents and include, for example, acetaminophen, non-steroidal anti-inflammatory drugs (NSAIDS) such as aspirin, ibuprofen, naproxen, etodolac (Lodine®) and celecoxib, colchicine (Colcrys®), corticosteroids such as prednisone, prednisolone, methylprednisolone, hydrocortisone, and the like, probenecid, allopurinol, febuxostat (Uloric®), sulfasalazine (Azulfidine®), antimalarials such as hydroxychloroquine (Plaquenil®) and chloroquine (Aralen®), methotrexate (Rheumatrex®), gold salts such as gold thioglucose (Solganal®), gold thiomalate (Myochrysine®) and auranofin (Ridaura®), D-penicillamine (Depen® or Cuprimine®), azathioprine (Imuran®), cyclophosphamide (Cytoxan®), chlorambucil (Leukeran®), cyclosporine (Sandimmune®), leflunomide (Arava®) and “anti-TNF” agents such as etanercept (Enbrel®), infliximab (Remicade®), golimumab (Simponi®), certolizumab pegol (Cimzia®) and adalimumab (Humira®), “anti-IL-1” agents such as anakinra (Kineret®) and rilonacept (Arcalyst®), canakinumab (Ilaris®), anti-Jak inhibitors such as tofacitinib, antibodies such as rituximab (Rituxan®), “anti-T-cell” agents such as abatacept (Orencia®), “anti-IL-6” agents such as tocilizumab (Actemra®), diclofenac, cortisone, hyaluronic acid (Synvisc® or Hyalgan®), monoclonal antibodies such as tanezumab, anticoagulants such as heparin (Calcinparine® or Liquaemin®) and warfarin (Coumadin®), antidiarrheals such as diphenoxylate (Lomotil®) and loperamide (Imodium®), bile acid binding agents such as cholestyramine, alosetron (Lotronex®), lubiprostone (Amitiza®), laxatives such as Milk of Magnesia, polyethylene glycol (MiraLax®), Dulcolax®, Correctol® and Senokot®, anticholinergics or antispasmodics such as dicyclomine (Bentyl®), Singulair®, beta-2 agonists such as albuterol (Ventolin® HFA, Proventil® HFA), levalbuterol (Xopenex®), metaproterenol (Alupent®), pirbuterol acetate (Maxair®), terbutaline sulfate (Brethaire®), salmeterol xinafoate (Serevent®) and formoterol (Foradil®), anticholinergic agents such as ipratropium bromide (Atrovent®) and tiotropium (Spiriva®), inhaled corticosteroids such as beclomethasone dipropionate (Beclovent®, Qvar®, and Vanceril®), triamcinolone acetonide (Azmacort®), mometasone (Asthmanex®), budesonide (Pulmocort®), and flunisolide (Aerobid®), Afviar®, Symbicort®, Dulera®, cromolyn sodium (Intal®), methylxanthines such as theophylline (Theo-Dur®, Theolair®, Slo-bid®, Uniphyl®, Theo-24®) and aminophylline, IgE antibodies such as omalizumab (Xolair®), nucleoside reverse transcriptase inhibitors such as zidovudine (Retrovir®), abacavir (Ziagen®), abacavir/lamivudine (Epzicom®), abacavir/lamivudine/zidovudine (Trizivir®), didanosine (Videx®), emtricitabine (Emtriva®), lamivudine (Epivir®), lamivudine/zidovudine (Combivir®), stavudine (Zerit®), and zalcitabine (Hivid®), non-nucleoside reverse transcriptase inhibitors such as delavirdine (Rescriptor®), efavirenz (Sustiva®), nevairapine (Viramune®) and etravirine (Intelence®), nucleotide reverse transcriptase inhibitors such as tenofovir (Viread®), protease inhibitors such as amprenavir (Agenerase®), atazanavir (Reyataz®), darunavir (Prezista®), fosamprenavir (Lexiva®), indinavir (Crixivan®), lopinavir and ritonavir (Kaletra®), nelfinavir (Viracept®), ritonavir (Norvir®), saquinavir (Fortovase® or Invirase®), and tipranavir (Aptivus®), entry inhibitors such as enfuvirtide (Fuzeon®) and maraviroc (Selzentry®), integrase inhibitors such as raltegravir (Isentress®), doxorubicin (Hydrodaunorubicin®), vincristine (Oncovin®), bortezomib (Velcade®), and dexamethasone (Decadron®) in combination with lenalidomide (Revlimid®), or any combination(s) thereof.

In another embodiment, described is a method of treating rheumatoid arthritis comprising administering to a patient in need thereof Compound 1 and one or more additional therapeutic agents selected from non-steroidal anti-inflammatory drugs (NSAIDS) such as aspirin, ibuprofen, naproxen, etodolac (Lodine®) and celecoxib, corticosteroids such as prednisone, prednisolone, methylprednisolone, hydrocortisone, and the like, sulfasalazine (Azulfidine®), antimalarials such as hydroxychloroquine (Plaquenil®) and chloroquine (Aralen®), methotrexate (Rheumatrex®), gold salts such as gold thioglucose (Solganal®), gold thiomalate (Myochrysine®) and auranofin (Ridaura®), D-penicillamine (Depen® or Cuprimine®), azathioprine (Imuran®), cyclophosphamide (Cytoxan®), chlorambucil (Leukeran®), cyclosporine (Sandimmune®), leflunomide (Arava®) and “anti-TNF” agents such as etanercept (Enbrel®), infliximab (Remicade®), golimumab (Simponi®), certolizumab pegol (Cimzia®) and adalimumab (Humira®), “anti-IL-1” agents such as anakinra (Kineret®) and rilonacept (Arcalyst®), antibodies such as rituximab (Rituxan®), “anti-T-cell” agents such as abatacept (Orencia®) and “anti-IL-6” agents such as tocilizumab (Actemra®).

In some embodiments, described is a method of treating osteoarthritis comprising administering to a patient in need thereof Compound 1 and one or more additional therapeutic agents selected from acetaminophen, non-steroidal anti-inflammatory drugs (NSAIDS) such as aspirin, ibuprofen, naproxen, etodolac (Lodine®) and celecoxib, diclofenac, cortisone, hyaluronic acid (Synvisc® or Hyalgan®) and monoclonal antibodies such as tanezumab.

In some embodiments, described is a method of treating cutaneous lupus erythematosus or systemic lupus erythematosus comprising administering to a patient in need thereof Compound 1 and one or more additional therapeutic agents selected from acetaminophen, non-steroidal anti-inflammatory drugs (NSAIDS) such as aspirin, ibuprofen, naproxen, etodolac (Lodine®) and celecoxib, corticosteroids such as prednisone, prednisolone, methylprednisolone, hydrocortisone, and the like, antimalarials such as hydroxychloroquine (Plaquenil®) and chloroquine (Aralen®), cyclophosphamide (Cytoxan®), methotrexate (Rheumatrex®), azathioprine (Imuran®) and anticoagulants such as heparin (Calcinparine® or Liquaemin®) and warfarin (Coumadin®).

In some embodiments, described is a method of treating Crohn's disesase, ulcerative colitis, or inflammatory bowel disease comprising administering to a patient in need thereof. Compound 1 and one or more additional therapeutic agents selected from mesalamine (Asacol®) sulfasalazine (Azulfidine®), antidiarrheals such as diphenoxylate (Lomotil®) and loperamide (Imodium®), bile acid binding agents such as cholestyramine, alosetron (Lotronex®), lubiprostone (Amitiza®), laxatives such as Milk of Magnesia, polyethylene glycol (MiraLax®), Dulcolax®, Correctol® and Senokot® and anticholinergics or antispasmodics such as dicyclomine (Bentyl®), anti-TNF therapies, steroids, and antibiotics such as Flagyl or ciprofloxacin.

In some embodiments, described is a method of treating asthma comprising administering to a patient in need thereof Compound 1 and one or more additional therapeutic agents selected from Singulair®, beta-2 agonists such as albuterol (Ventolin® HFA, Proventil® HFA), levalbuterol (Xopenex®), metaproterenol (Alupent®), pirbuterol acetate (Maxair®), terbutaline sulfate (Brethaire®), salmeterol xinafoate (Serevent®) and formoterol (Foradil®), anticholinergic agents such as ipratropium bromide (Atrovent®) and tiotropium (Spiriva®), inhaled corticosteroids such as prednisone, prednisolone, beclomethasone dipropionate (Beclovent®, Qvar®, and Vanceril®), triamcinolone acetonide (Azmacort®), mometasone (Asthmanex®), budesonide (Pulmocort®), flunisolide (Aerobid®), Afviar®, Symbicort®, and Dulera®, cromolyn sodium (Intal®), methylxanthines such as theophylline (Theo-Dur®, Theolair®, Slo-bid®, Uniphyl®, Theo-24®) and aminophylline, and IgE antibodies such as omalizumab (Xolair®).

In some embodiments, described is a method of treating COPD comprising administering to a patient in need thereof Compound 1 and one or more additional therapeutic agents selected from beta-2 agonists such as albuterol (Ventolin® HFA, Proventil® HFA), levalbuterol (Xopenex®), metaproterenol (Alupent®), pirbuterol acetate (Maxair®), terbutaline sulfate (Brethaire®), salmeterol xinafoate (Serevent®) and formoterol (Foradil®), anticholinergic agents such as ipratropium bromide (Atrovent®) and tiotropium (Spiriva®), methylxanthines such as theophylline (Theo-Dur®, Theolair®, Slo-bid®, Uniphyl®, Theo-24®) and aminophylline, inhaled corticosteroids such as prednisone, prednisolone, beclomethasone dipropionate (Beclovent®, Qvar®, and Vanceril®), triamcinolone acetonide (Azmacort®), mometasone (Asthmanex®), budesonide (Pulmocort®), flunisolide (Aerobid®), Afviar®, Symbicort®, and Dulera®,

In another embodiment, described is a method of treating a hematological malignancy comprising administering to a patient in need thereof Compound 1 and one or more additional therapeutic agents selected from rituximab (Rituxan®), cyclophosphamide (Cytoxan®), doxorubicin (Hydrodaunorubicin®), vincristine (Oncovin®), prednisone, a hedgehog signaling inhibitor, a BTK inhibitor, a JAK/pan-JAK inhibitor, a PI3K inhibitor, a SYK inhibitor, and combinations thereof.

In another embodiment, described is a method of treating a solid tumor comprising administering to a patient in need thereof Compound 1 and one or more additional therapeutic agents selected from rituximab (Rituxan®), cyclophosphamide (Cytoxan®), doxorubicin (Hydrodaunorubicin®), vincristine (Oncovin®), prednisone, a hedgehog signaling inhibitor, a BTK inhibitor, a JAK/pan-JAK inhibitor, a PI3K inhibitor, a SYK inhibitor, and combinations thereof.

In another embodiment, described is a method of treating a hematological malignancy comprising administering to a patient in need thereof Compound 1 and a Hedgehog (Hh) signaling pathway inhibitor. In some embodiments, the hematological malignancy is DLBCL (Ramirez et al “Defining causative factors contributing in the activation of hedgehog signaling in diffuse large B-cell lymphoma” Leuk. Res. (2012), published online July 17, and incorporated herein by reference in its entirety).

In another embodiment, described is a method of treating diffuse large B-cell lymphoma (DLBCL) comprising administering to a patient in need thereof Compound 1 and one or more additional therapeutic agents selected from rituximab (Rituxan®), cyclophosphamide (Cytoxan®), doxorubicin (Hydrodaunorubicin®), vincristine (Oncovin®), prednisone, a hedgehog signaling inhibitor, and combinations thereof.

In another embodiment, described is a method of treating multiple myeloma comprising administering to a patient in need thereof a compound of Compound 1 and one or more additional therapeutic agents selected from bortezomib (Velcade®), and dexamethasone (Decadron®), a hedgehog signaling inhibitor, a BTK inhibitor, a JAK/pan-JAK inhibitor, a TYK2 inhibitor, a PI3K inhibitor, a SYK inhibitor in combination with lenalidomide (Revlimid®).

In another embodiment, described is a method of treating or lessening the severity of a disease comprising administering to a patient in need thereof Compound 1 and a BTK inhibitor, wherein the disease is selected from inflammatory bowel disease, arthritis, cutaneous lupus erythematosus, systemic lupus erythematosus (SLE), vasculitis, idiopathic thrombocytopenic purpura (ITP), rheumatoid arthritis, psoriatic arthritis, osteoarthritis, Still's disease, juvenile arthritis, diabetes, myasthenia gravis, Hashimoto's thyroiditis, Ord's thyroiditis, Graves' disease, autoimmune thyroiditis, Sjogren's syndrome, multiple sclerosis, systemic sclerosis, Lyme neuroborreliosis, Guillain-Barre syndrome, acute disseminated encephalomyelitis, Addison's disease, opsoclonus-myoclonus syndrome, ankylosing spondylosis, antiphospholipid antibody syndrome, aplastic anemia, autoimmune hepatitis, autoimmune gastritis, pernicious anemia, celiac disease, Goodpasture's syndrome, idiopathic thrombocytopenic purpura, optic neuritis, scleroderma, primary biliary cirrhosis, Reiter's syndrome, Takayasu's arteritis, temporal arteritis, warm autoimmune hemolytic anemia, Wegener's granulomatosis, psoriasis, alopecia universalis, Behcet's disease, chronic fatigue, dysautonomia, membranous glomerulonephropathy, endometriosis, interstitial cystitis, pemphigus vulgaris, bullous pemphigoid, neuromyotonia, scleroderma, vulvodynia, a hyperproliferative disease, rejection of transplanted organs or tissues, Acquired Immunodeficiency Syndrome (AIDS, also known as HIV), type 1 diabetes, graft versus host disease, transplantation, transfusion, anaphylaxis, allergies (e.g., allergies to plant pollens, latex, drugs, foods, insect poisons, animal hair, animal dander, dust mites, or cockroach calyx), type I hypersensitivity, allergic conjunctivitis, allergic rhinitis, and atopic dermatitis, asthma, appendicitis, atopic dermatitis, asthma, allergy, blepharitis, bronchiolitis, bronchitis, bursitis, cervicitis, cholangitis, cholecystitis, chronic graft rejection, colitis, conjunctivitis, Crohn's disease, cystitis, dacryoadenitis, dermatitis, dermatomyositis, encephalitis, endocarditis, endometritis, enteritis, enterocolitis, epicondylitis, epididymitis, fasciitis, fibrositis, gastritis, gastroenteritis, Henoch-Schonlein purpura, hepatitis, hidradenitis suppurativa, immunoglobulin A nephropathy, interstitial lung disease, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, polymyositis, proctitis, prostatitis, pyelonephritis, rhinitis, salpingitis, sinusitis, stomatitis, synovitis, tendonitis, tonsillitis, ulcerative colitis, uveitis, vaginitis, vasculitis, or vulvitis, B-cell proliferative disorder, e.g., diffuse large B cell lymphoma, follicular lymphoma, chronic lymphocytic lymphoma, chronic lymphocytic leukemia, acute lymphocytic leukemia, B-cell prolymphocytic leukemia, lymphoplasmacytic lymphoma/Waldenstrom macroglobulinemia, splenic marginal zone lymphoma, multiple myeloma (also known as plasma cell myeloma), non-Hodgkin's lymphoma, Hodgkin's lymphoma, plasmacytoma, extranodal marginal zone B cell lymphoma, nodal marginal zone B cell lymphoma, mantle cell lymphoma, mediastinal (thymic) large B cell lymphoma, intravascular large B cell lymphoma, primary effusion lymphoma, Burkitt lymphoma/leukemia, or lymphomatoid granulomatosis, breast cancer, prostate cancer, or cancer of the mast cells (e.g., mastocytoma, mast cell leukemia, mast cell sarcoma, systemic mastocytosis), bone cancer, colorectal cancer, pancreatic cancer, diseases of the bone and joints including, without limitation, rheumatoid arthritis, seronegative spondyloarthropathies (including ankylosing spondylitis, psoriatic arthritis and Reiter's disease), systemic sclerosis, osteoporosis, bone cancer, bone metastasis, a thromboembolic disorder, (e.g., myocardial infarct, angina pectoris, reocclusion after angioplasty, restenosis after angioplasty, reocclusion after aortocoronary bypass, restenosis after aortocoronary bypass, stroke, transitory ischemia, a peripheral arterial occlusive disorder, pulmonary embolism, deep venous thrombosis), inflammatory pelvic disease, urethritis, skin sunburn, sinusitis, pneumonitis, encephalitis, meningitis, myocarditis, nephritis, osteomyelitis, myositis, hepatitis, gastritis, enteritis, dermatitis, gingivitis, appendicitis, pancreatitis, cholocystitus, agammaglobulinemia, psoriasis, allergy, Crohn's disease, irritable bowel syndrome, ulcerative colitis, Sjogren's disease, tissue graft rejection, hyperacute rejection of transplanted organs, asthma, allergic rhinitis, chronic obstructive pulmonary disease (COPD), autoimmune polyglandular disease (also known as autoimmune polyglandular syndrome), autoimmune alopecia, pernicious anemia, glomerulonephritis, dermatomyositis, multiple sclerosis, scleroderma, vasculitis, autoimmune hemolytic and thrombocytopenic states, Goodpasture's syndrome, atherosclerosis, Addison's disease, Parkinson's disease, Alzheimer's disease, diabetes, septic shock, cutaneous lupus erythematosus, systemic lupus erythematosus (SLE), rheumatoid arthritis, psoriatic arthritis, juvenile arthritis, osteoarthritis, chronic idiopathic thrombocytopenic purpura, myasthenia gravis, Hashimoto's thyroiditis, atopic dermatitis, degenerative joint disease, vitiligo, autoimmune hypopituitarism, scleroderma, mycosis fungoides, and acute inflammatory responses (such as acute respiratory distress syndrome and ischemia/reperfusion injury).

In another embodiment, described is a method of treating or lessening the severity of a disease comprising administering to a patient in need thereof Compound 1 prepared according to the method disclosed herein, and a PI3K inhibitor, wherein the disease is selected from a cancer, a neurodegenative disorder, an angiogenic disorder, a viral disease, an autoimmune disease, an inflammatory disorder, a hormone-related disease, conditions associated with organ transplantation, immunodeficiency disorders, a destructive bone disorder, a proliferative disorder, an infectious disease, a condition associated with cell death, thrombin-induced platelet aggregation, chronic myelogenous leukemia (CML), chronic lymphocytic leukemia (CLL), liver disease, pathologic immune conditions involving T cell activation, a cardiovascular disorder, and a CNS disorder.

In another embodiment, described is a method of treating or lessening the severity of a disease comprising administering to a patient in need thereof Compound 1 prepared according to the method disclosed herein, and a PI3K inhibitor, wherein the disease is selected from benign or malignant tumor, carcinoma or solid tumor of the brain, kidney (e.g., renal cell carcinoma (RCC)), liver, adrenal gland, bladder, breast, stomach, gastric tumors, ovaries, colon, rectum, prostate, pancreas, lung, vagina, endometrium, cervix, testis, genitourinary tract, esophagus, larynx, skin, bone or thyroid, sarcoma, glioblastomas, neuroblastomas, multiple myeloma or gastrointestinal cancer, especially colon carcinoma or colorectal adenoma or a tumor of the neck and head, an epidermal hyperproliferation, psoriasis, prostate hyperplasia, a neoplasia, a neoplasia of epithelial character, adenoma, adenocarcinoma, keratoacanthoma, epidermoid carcinoma, large cell carcinoma, non-small-cell lung carcinoma, lymphomas, (including, for example, non-Hodgkin's Lymphoma (NHL) and Hodgkin's lymphoma (also termed Hodgkin's or Hodgkin's disease)), a mammary carcinoma, follicular carcinoma, undifferentiated carcinoma, papillary carcinoma, seminoma, melanoma, or a leukemia, diseases include Cowden syndrome, Lhermitte-Dudos disease and Bannayan-Zonana syndrome, or diseases in which the PI3K/PKB pathway is aberrantly activated, asthma of whatever type or genesis including both intrinsic (non-allergic) asthma and extrinsic (allergic) asthma, mild asthma, moderate asthma, severe asthma, bronchitic asthma, exercise-induced asthma, occupational asthma and asthma induced following bacterial infection, acute lung injury (ALI), adult/acute respiratory distress syndrome (ARDS), chronic obstructive pulmonary, airways or lung disease (COPD, COAD or COLD), including chronic bronchitis or dyspnea associated therewith, emphysema, as well as exacerbation of airways hyperreactivity consequent to other drug therapy, in particular other inhaled drug therapy, bronchitis of whatever type or genesis including, but not limited to, acute, arachidic, catarrhal, croupus, chronic or phthinoid bronchitis, pneumoconiosis (an inflammatory, commonly occupational, disease of the lungs, frequently accompanied by airways obstruction, whether chronic or acute, and occasioned by repeated inhalation of dusts) of whatever type or genesis, including, for example, aluminosis, anthracosis, asbestosis, chalicosis, ptilosis, siderosis, silicosis, tabacosis and byssinosis, Loffler's syndrome, eosinophilic, pneumonia, parasitic (in particular metazoan) infestation (including tropical eosinophilia), bronchopulmonary aspergillosis, polyarteritis nodosa (including Churg-Strauss syndrome), eosinophilic granuloma and eosinophil-related disorders affecting the airways occasioned by drug-reaction, psoriasis, contact dermatitis, atopic dermatitis, alopecia areata, erythema multiforma, dermatitis herpetiformis, scleroderma, vitiligo, hypersensitivity angiitis, urticaria, bullous pemphigoid, lupus erythematosus, pemphisus, epidermolysis bullosa acquisita, conjunctivitis, keratoconjunctivitis sicca, and vernal conjunctivitis, diseases affecting the nose including allergic rhinitis, and inflammatory disease in which autoimmune reactions are implicated or having an autoimmune component or etiology, including autoimmune hematological disorders (e.g. hemolytic anemia, aplastic anemia, pure red cell anemia and idiopathic thrombocytopenia), cutaneous lupus erythematosus, systemic lupus erythematosus, rheumatoid arthritis, polychondritis, sclerodoma, Wegener granulamatosis, dermatomyositis, chronic active hepatitis, myasthenia gravis, Steven-Johnson syndrome, idiopathic sprue, autoimmune inflammatory bowel disease (e.g. ulcerative colitis and Crohn's disease), endocrine opthalmopathy, Grave's disease, sarcoidosis, alveolitis, chronic hypersensitivity pneumonitis, multiple sclerosis, primary biliary cirrhosis, uveitis (anterior and posterior), keratoconjunctivitis sicca and vernal keratoconjunctivitis, interstitial lung fibrosis, psoriatic arthritis and glomerulonephritis (with and without nephrotic syndrome, e.g. including idiopathic nephrotic syndrome or minal change nephropathy, restenosis, cardiomegaly, atherosclerosis, myocardial infarction, ischemic stroke and congestive heart failure, Alzheimer's disease, Parkinson's disease, amyotrophic lateral sclerosis, Huntington's disease, and cerebral ischemia, and neurodegenerative disease caused by traumatic injury, glutamate neurotoxicity and hypoxia.

In some embodiments described is a method of treating or lessening the severity of a disease comprising administering to a patient in need thereof Compound 1 prepared according to the method disclosed herein, and a Bcl-2 inhibitor, wherein the disease is an inflammatory disorder, an autoimmune disorder, a proliferative disorder, an endocrine disorder, a neurological disorder, or a disorder associated with transplantation. In some embodiments, the disorder is a proliferative disorder, lupus, or lupus nephritis. In some embodiments, the proliferative disorder is chronic lymphocytic leukemia, diffuse large B-cell lymphoma, Hodgkin's disease, small-cell lung cancer, non-small-cell lung cancer, myelodysplastic syndrome, lymphoma, a hematological neoplasm, or solid tumor.

In some embodiments, described is a method of treating or lessening the severity of a disease, comprising administering to a patient in need thereof a TYK2 pseudokinase (JH2) domain binding compound and a TYK2 kinase (JH1) domain binding compound. In some embodiments, the disease is an autoimmune disorder, an inflammatory disorder, a proliferative disorder, an endocrine disorder, a neurological disorder, or a disorder associated with transplantation. In some embodiments the JH2 binding Compound 1 prepared according to the method disclosed herein. Other suitable JH2 domain binding compounds include those described in WO2014074660A1, WO2014074661A1, WO2015089143A1, the entirety of each of which is incorporated herein by reference. Suitable JHT domain binding compounds include those described in WO2015131080A1, the entirety of which is incorporated herein by reference.

Compound 1 and compositions, according to the method described herein, may be administered using any amount and any route of administration effective for treating or lessening the severity of an autoimmune disorder, an inflammatory disorder, a proliferative disorder, an endocrine disorder, a neurological disorder, or a disorder associated with transplantation. The exact amount required will vary from subject to subject, depending on the species, age, and general condition of the subject, the severity of the infection, the particular agent, its mode of administration, and the like. The Compound herein is preferably formulated in dosage unit form for ease of administration and uniformity of dosage. The expression “dosage unit form” as used herein refers to a physically discrete unit of agent appropriate for the patient to be treated. It will be understood, however, that the total daily usage of the compound and compositions will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts. The term “patient”, as used herein, means an animal, preferably a mammal, and most preferably a human.

Pharmaceutically acceptable compositions can be administered to humans and other animals orally, rectally, parenterally, intracisternally, intravaginally, intraperitoneally, topically (as by powders, ointments, or drops), bucally, as an oral or nasal spray, or the like, depending on the severity of the infection being treated. In certain embodiments, the compound may be administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a Compound 1, it is may be desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of Compound 1 release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing Compound 1 prepared by the method with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compound can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of Compound 1 prepared by the method include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope. Additionally, contemplated is the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

According to one embodiment, described is a method of inhibiting protein kinase activity in a biological sample comprising the step of contacting said biological sample with Compound 1 prepared by the method, or a composition comprising said compound.

According to another embodiment, a method of inhibiting TYK2, or a mutant thereof, activity in a biological sample comprising the step of contacting said biological sample with a compound, or a composition comprising said compound is envisaged. In certain embodiments, described is a method of irreversibly inhibiting TYK2, or a mutant thereof, activity in a biological sample comprising the step of contacting said biological sample with Compound 1 prepared by the methods herein, or a composition comprising said compound.

In another embodiment, described is a method of selectively inhibiting TYK2 over one or more of JAK1, JAK2, and JAK3. In some embodiments, Compound 1 produced by the method herein is more than 2-fold selective over JAK1/2/3. In some embodiments, the compound is more than 5-fold selective over JAK1/2/3. In some embodiments, the compound is more than 10-fold selective over JAK1/2/3. In some embodiments, the compound is more than 50-fold selective over JAK1/2/3. In some embodiments, the compound is more than 100-fold selective over JAK1/2/3.

The term “biological sample”, as used herein, includes, without limitation, cell cultures or extracts thereof; biopsied material obtained from a mammal or extracts thereof; and blood, saliva, urine, feces, semen, tears, or other body fluids or extracts thereof.

Inhibition of TYK2 (or a mutant thereof) activity in a biological sample is useful for a variety of purposes that are known to one of skill in the art. Examples of such purposes include, but are not limited to, blood transfusion, organ-transplantation, biological specimen storage, and biological assays.

Another embodiment relates to a method of inhibiting protein kinase activity in a patient comprising the step of administering to said patient Compound 1 prepared by the method herein, or a composition comprising said compound.

Another embodiment relates to a method of inhibiting activity of TYK2, or a mutant thereof, in a patient comprising the step of administering to said patient Compound 1 prepared by the method herein, or a composition comprising said compound. According to certain embodiments, a method of reversibly or irreversibly inhibiting one or more of TYK2, or a mutant thereof, activity in a patient comprising the step of administering to said patient Compound 1 prepared by the method herein, or a composition comprising said compound. In other embodiments, a method is provided for treating a disorder mediated by TYK2, or a mutant thereof, in a patient in need thereof, comprising the step of administering to said patient Compound 1 prepared by the method herein or pharmaceutically acceptable composition thereof. Such disorders are described in detail herein.

Depending upon the particular condition, or disease, to be treated, additional therapeutic agents that are normally administered to treat that condition, may also be present in the compositions. As used herein, additional therapeutic agents that are normally administered to treat a particular disease, or condition, are known as “appropriate for the disease, or condition, being treated.”

Compound 1 prepared by the method herein may also be used to advantage in combination with other therapeutic compounds. In some embodiments, the other therapeutic compounds are antiproliferative compounds. Such antiproliferative compounds include, but are not limited to aromatase inhibitors; antiestrogens; topoisomerase I inhibitors; topoisomerase IT inhibitors; microtubule active compounds; alkylating compounds; histone deacetylase inhibitors; compounds which induce cell differentiation processes; cyclooxygenase inhibitors; MMP inhibitors; mTOR inhibitors; antineoplastic antimetabolites; platin compounds; compounds targeting/decreasing a protein or lipid kinase activity and further anti-angiogenic compounds; compounds which target, decrease or inhibit the activity of a protein or lipid phosphatase; gonadorelin agonists; anti-androgens; methionine aminopeptidase inhibitors; matrix metalloproteinase inhibitors; bisphosphonates; biological response modifiers; antiproliferative antibodies; heparanase inhibitors; inhibitors of Ras oncogenic isoforms; telomerase inhibitors; proteasome inhibitors; compounds used in the treatment of hematologic malignancies; compounds which target, decrease or inhibit the activity of Flt-3; Hsp90 inhibitors such as 17-AAG (17-allylaminogeldanamycin, NSC330507), 17-DMAG (17-dimethylaminoethylamino-17-demethoxy-geldanamycin, NSC707545), IPI-504, CNF1010, CNF2024, CNF1010 from Conforma Therapeutics; temozolomide (Temodal®); kinesin spindle protein inhibitors, such as SB715992 or SB743921 from GlaxoSmithKline, or pentamidine/chlorpromazine from CombinatoRx; MEK inhibitors such as ARRY142886 from Array BioPharma, AZD6244 from AstraZeneca, PD181461 from Pfizer and leucovorin. The term “aromatase inhibitor” as used herein relates to a compound which inhibits estrogen production, for instance, the conversion of the substrates androstenedione and testosterone to estrone and estradiol, respectively. The term includes, but is not limited to steroids, especially atamestane, exemestane and formestane and, in particular, non-steroids, especially aminoglutethimide, roglethimide, pyridoglutethimide, trilostane, testolactone, ketokonazole, vorozole, fadrozole, anastrozole and letrozole. Exemestane is marketed under the trade name Aromasin™. Formestane is marketed under the trade name Lentaron™. Fadrozole is marketed under the trade name Afema™. Anastrozole is marketed under the trade name Arimidex™. Letrozole is marketed under the trade names Femara™ or Femar™ Aminoglutethimide is marketed under the trade name Orimeten™. A combination comprising a chemotherapeutic agent which is an aromatase inhibitor is particularly useful for the treatment of hormone receptor positive tumors, such as breast tumors.

The term “antiestrogen” as used herein relates to a compound which antagonizes the effect of estrogens at the estrogen receptor level. The term includes, but is not limited to tamoxifen, fulvestrant, raloxifene and raloxifene hydrochloride. Tamoxifen is marketed under the trade name Nolvadex™. Raloxifene hydrochloride is marketed under the trade name Evista™. Fulvestrant can be administered under the trade name Faslodex™. A combination comprising a chemotherapeutic agent which is an antiestrogen is particularly useful for the treatment of estrogen receptor positive tumors, such as breast tumors.

The term “anti-androgen” as used herein relates to any substance which is capable of inhibiting the biological effects of androgenic hormones and includes, but is not limited to, bicalutamide (Casodex™). The term “gonadorelin agonist” as used herein includes, but is not limited to abarelix, goserelin and goserelin acetate. Goserelin can be administered under the trade name Zoladex™.

The term “topoisomerase I inhibitor” as used herein includes, but is not limited to topotecan, gimatecan, irinotecan, camptothecian and its analogues, 9-nitrocamptothecin and the macromolecular camptothecin conjugate PNU-166148. Irinotecan can be administered, e.g. in the form as it is marketed, e.g. under the trademark Camptosar™. Topotecan is marketed under the trade name Hycamptin™.

The term “topoisomerase II inhibitor” as used herein includes, but is not limited to the anthracyclines such as doxorubicin (including liposomal formulation, such as Caelyx™) daunorubicin, epirubicin, idarubicin and nemorubicin, the anthraquinones mitoxantrone and losoxantrone, and the podophillotoxines etoposide and teniposide. Etoposide is marketed under the trade name Etopophos™. Teniposide is marketed under the trade name VM 26-Bristol Doxorubicin is marketed under the trade name Acriblastin™ or Adriamycin™. Epirubicin is marketed under the trade name Farmorubicin™. Idarubicin is marketed. under the trade name Zavedos™. Mitoxantrone is marketed under the trade name Novantron.

The term “microtubule active agent” relates to microtubule stabilizing, microtubule destabilizing compounds and microtublin polymerization inhibitors including, but not limited to taxanes, such as paclitaxel and docetaxel; vinca alkaloids, such as vinblastine or vinblastine sulfate, vincristine or vincristine sulfate, and vinorelbine; discodermolides; cochicine and epothilones and derivatives thereof. Paclitaxel is marketed under the trade name Taxol™ Docetaxel is marketed under the trade name Taxotere™. Vinblastine sulfate is marketed under the trade name Vinblastin R.P™. Vincristine sulfate is marketed under the trade name Farmistin™.

The term “alkylating agent” as used herein includes, but is not limited to, cyclophosphamide, ifosfamide, melphalan or nitrosourea (BCNU or Gliadel). Cyclophosphamide is marketed under the trade name Cyclostin™. Ifosfamide is marketed under the trade name Holoxan™.

The term “histone deacetylase inhibitors” or “HDAC inhibitors” relates to compounds which inhibit the histone deacetylase and which possess antiproliferative activity. This includes, but is not limited to, suberoylanilide hydroxamic acid (SAHA).

The term “antineoplastic antimetabolite” includes, but is not limited to, 5-fluorouracil or 5-FU, capecitabine, gemcitabine, DNA demethylating compounds, such as 5-azacytidine and decitabine, methotrexate and edatrexate, and folic acid antagonists such as pemetrexed. Capecitabine is marketed under the trade name Xeloda™. Gemcitabine is marketed under the trade name Gemzar™.

The term “platin compound” as used herein includes, but is not limited to, carboplatin, cis-platin, cisplatinum and oxaliplatin. Carboplatin can be administered, e.g., in the form as it is marketed, e.g. under the trademark Carboplat™. Oxaliplatin can be administered, e.g., in the form as it is marketed, e.g. under the trademark Eloxatin™.

The term “compounds targeting/decreasing a protein or lipid kinase activity; or a protein or lipid phosphatase activity; or further anti-angiogenic compounds” as used herein includes, but is not limited to, protein tyrosine kinase and/or serine and/or threonine kinase inhibitors or lipid kinase inhibitors, such as a) compounds targeting, decreasing or inhibiting the activity of the platelet-derived growth factor-receptors (PDGFR), such as compounds which target, decrease or inhibit the activity of PDGFR, especially compounds which inhibit the PDGF receptor, such as an N-phenyl-2-pyrimidine-amine derivative, such as imatinib, SU101, SU6668 and GFB-111; b) compounds targeting, decreasing or inhibiting the activity of the fibroblast growth factor-receptors (FGFR); c) compounds targeting, decreasing or inhibiting the activity of the insulin-like growth factor receptor I (IGF-IR), such as compounds which target, decrease or inhibit the activity of IGF-IR, especially compounds which inhibit the kinase activity of IGF-I receptor, or antibodies that target the extracellular domain of IGF-I receptor or its growth factors; d) compounds targeting, decreasing or inhibiting the activity of the Trk receptor tyrosine kinase family, or ephrin B4 inhibitors; e) compounds targeting, decreasing or inhibiting the activity of the Ax1 receptor tyrosine kinase family; f) compounds targeting, decreasing or inhibiting the activity of the Ret receptor tyrosine kinase; g) compounds targeting, decreasing or inhibiting the activity of the Kit/SCFR receptor tyrosine kinase, such as imatinib; h) compounds targeting, decreasing or inhibiting the activity of the C-kit receptor tyrosine kinases, which are part of the PDGFR family, such as compounds which target, decrease or inhibit the activity of the c-Kit receptor tyrosine kinase family, especially compounds which inhibit the c-Kit receptor, such as imatinib; i) compounds targeting, decreasing or inhibiting the activity of members of the c-Abl family, their gene-fusion products (e.g. BCR-Abl kinase) and mutants, such as compounds which target decrease or inhibit the activity of c-Abl family members and their gene fusion products, such as an N-phenyl-2-pyrimidine-amine derivative, such as imatinib or nilotinib (AMN107); PD180970; AG957; NSC 680410; PD173955 from ParkeDavis; or dasatinib (BMS-354825); j) compounds targeting, decreasing or inhibiting the activity of members of the protein kinase C (PKC) and Raf family of serine/threonine kinases, members of the MEK, SRC, JAK/pan-JAK, FAK, PDK1, PKB/Akt, Ras/MAPK, PI3K, SYK, BTK and TEC family, and/or members of the cyclin-dependent kinase family (CDK) including staurosporine derivatives, such as midostaurin; examples of further compounds include UCN-01, safingol, BAY 43-9006, Bryostatin 1, Perifosine; llmofosine; RO 318220 and RO 320432; GO 6976; 1sis 3521; LY333531/LY379196; isochinoline compounds; FTIs; PD184352 or QAN697 (a PI3K inhibitor) or AT7519 (CDK inhibitor); k) compounds targeting, decreasing or inhibiting the activity of protein-tyrosine kinase inhibitors, such as compounds which target, decrease or inhibit the activity of protein-tyrosine kinase inhibitors include imatinib mesylate (Gleevec™) or tyrphostin such as Tyrphostin A23/RG-50810; AG 99; Tyrphostin AG 213; Tyrphostin AG 1748; Tyrphostin AG 490; Tyrphostin B44; Tyrphostin B44 (+) enantiomer; Tyrphostin AG 555; AG 494; Tyrphostin AG 556, AG957 and adaphostin (4-{[(2,5-dihydroxyphenyl)methyl]amino}-benzoic acid adamantyl ester; NSC 680410, adaphostin); 1) compounds targeting, decreasing or inhibiting the activity of the epidermal growth factor family of receptor tyrosine kinases (EGFR1 ErbB2, ErbB3, ErbB4 as homo- or heterodimers) and their mutants, such as compounds which target, decrease or inhibit the activity of the epidermal growth factor receptor family are especially compounds, proteins or antibodies which inhibit members of the EGF receptor tyrosine kinase family, such as EGF receptor, ErbB2, ErbB3 and ErbB4 or bind to EGF or EGF related ligands, CP 358774, ZD 1839, ZM 105180; trastuzumab (Herceptin™), cetuximab (Erbitux™), Iressa, Tarceva, OSI-774, Cl-1033, EKB-569, GW-2016, E1.1, E2.4, E2.5, E6.2, E6.4, E2.11, E6.3 or E7.6.3, and 7H-pyrrolo-[2,3-d]pyrimidine derivatives; m) compounds targeting, decreasing or inhibiting the activity of the c-Met receptor, such as compounds which target, decrease or inhibit the activity of c-Met, especially compounds which inhibit the kinase activity of c-Met receptor, or antibodies that target the extracellular domain of c-Met or bind to HGF, n) compounds targeting, decreasing or inhibiting the kinase activity of one or more JAK family members (JAK1/JAK2/JAK3/TYK2 and/or pan-JAK), including but not limited to PRT-062070, SB-1578, baricitinib, pacritinib, momelotinib, VX-509, AZD-1480, TG-101348, tofacitinib, and ruxolitinib; o) compounds targeting, decreasing or inhibiting the kinase activity of PI3 kinase (P1³K) including but not limited to ATU-027, SF-1126, DS-7423, PBI-05204, GSK-2126458, ZSTK-474, buparlisib, pictrelisib, PF-4691502, BYL-719, dactolisib, XL-147, XL-765, and idelalisib; and; and q) compounds targeting, decreasing or inhibiting the signaling effects of hedgehog protein (Hh) or smoothened receptor (SMO) pathways, including but not limited to cyclopamine, vismodegib, itraconazole, erismodegib, and IPI-926 (saridegib).

The term “PI3K inhibitor” as used herein includes, but is not limited to compounds having inhibitory activity against one or more enzymes in the phosphatidylinositol-3-kinase family, including, but not limited to PI3Kα, PI3Kγ, PI3Kδ, PI3Kβ, PI3K-C2α, PI3K-C2α, PI3K-C2γ, Vps34, p110-α, p110-β, p110-γ, p110-δ, p85-α, p85-β, p55-γ, p150, p101, and p87. Examples of PI3K inhibitors useful include but are not limited to ATU-027, SF-1126, DS-7423, PBI-05204, GSK-2126458, ZSTK-474, buparlisib, pictrelisib, PF-4691502, BYL-719, dactolisib, XL-147, XL-765, and idelalisib.

The term “BTK inhibitor” as used herein includes, but is not limited to compounds having inhibitory activity against Bruton's Tyrosine Kinase (BTK), including, but not limited to AVL-292 and ibrutinib.

The term “SYK inhibitor” as used herein includes, but is not limited to compounds having inhibitory activity against spleen tyrosine kinase (SYK), including but not limited to PRT-062070, R-343, R-333, Excellair, PRT-062607, and fostamatinib.

The term “Bcl-2 inhibitor” as used herein includes, but is not limited to compounds having inhibitory activity against B-cell lymphoma 2 protein (Bel-2), including but not limited to ABT-199, ABT-731, ABT-737, apogossypol, Ascenta's pan-Bcl-2 inhibitors, curcumin (and analogs thereof), dual Bcl-2/Bcl-xL inhibitors (Infinity Pharmaceuticals/Novartis Pharmaceuticals), Genasense (G3139), HA14-1 (and analogs thereof; see WO2008118802), navitoclax (and analogs thereof, see U.S. Pat. No. 7,390,799), NH-1 (Shenayng Pharmaceutical University), obatoclax (and analogs thereof, see WO2004106328), S-001 (Gloria Pharmaceuticals), TW series compounds (Univ. of Michigan), and venetoclax. In some embodiments the Bcl-2 inhibitor is a small molecule therapeutic. In some embodiments the Bcl-2 inhibitor is a peptidomimetic.

Further examples of BTK inhibitory compounds, and conditions treatable by such compounds in combination with compounds herein can be found in WO2008039218 and WO2011090760, the entirety of which are incorporated herein by reference.

Further examples of SYK inhibitory compounds, and conditions treatable by such compounds in combination with compounds herein can be found in WO2003063794, WO2005007623, and WO2006078846, the entirety of which are incorporated herein by reference.

Further examples of PI3K inhibitory compounds, and conditions treatable by such compounds in combination with compounds herein can be found in WO2004019973, WO2004089925, WO2007016176, U.S. Pat. No. 8,138,347, WO2002088112, WO2007084786, WO2007129161, WO2006122806, WO2005113554, and WO2007044729 the entirety of which are incorporated herein by reference.

Further examples of JAK inhibitory compounds, and conditions treatable by such compounds in combination with compounds herein can be found in WO2009114512, WO2008109943, WO2007053452, WO2000142246, and WO2007070514, the entirety of which are incorporated herein by reference.

Further anti-angiogenic compounds include compounds having another mechanism for their activity, e.g. unrelated to protein or lipid kinase inhibition e.g. thalidomide (Thalomid™) and TNP-470.

Examples of proteasome inhibitors useful for use in combination Compound 1 prepared by the method herein include, but are not limited to bortezomib, disulfiram, epigallocatechin-3-gallate (EGCG), salinosporamide A, carfilzomib, ONX-0912, CEP-18770, and MLN9708.

Compounds which target, decrease or inhibit the activity of a protein or lipid phosphatase are e.g. inhibitors of phosphatase 1, phosphatase 2A, or CDC25, such as okadaic acid or a derivative thereof.

Compounds which induce cell differentiation processes include, but are not limited to, retinoic acid, α- γ- or δ-tocopherol or α- γ- or δ-tocotrienol.

The term cyclooxygenase inhibitor as used herein includes, but is not limited to, Cox-2 inhibitors, 5-alkyl substituted 2-arylaminophenylacetic acid and derivatives, such as celecoxib (Celebrex™), etoricoxib, valdecoxib or a 5-alkyl-2-arylaminophenylacetic acid, such as 5-methyl-2-(2′-chloro-6′-fluoroanilino)phenyl acetic acid, lumiracoxib.

The term “bisphosphonates” as used herein includes, but is not limited to, etridonic, clodronic, tiludronic, pamidronic, alendronic, ibandronic, risedronic and zoledronic acid. Etridonic acid is marketed under the trade name Didronel™. Clodronic acid is marketed under the trade name Bonefos™. Tiludronic acid is marketed under the trade name Skelid™. Pamidronic acid is marketed under the trade name Aredia™. Alendronic acid is marketed under the trade name Fosamax™. Ibandronic acid is marketed under the trade name Bondranat™. Risedronic acid is marketed under the trade name Actonel™. Zoledronic acid is marketed under the trade name Zometa™. The term “mTOR inhibitors” relates to compounds which inhibit the mammalian target of rapamycin (mTOR) and which possess antiproliferative activity such as sirolimus (Rapamune®), everolimus (Certican™), CCI-779 and ABT578.

The term “heparanase inhibitor” as used herein refers to compounds which target, decrease or inhibit heparin sulfate degradation. The term includes, but is not limited to, PI-88. The term “biological response modifier” as used herein refers to a lymphokine or interferons.

The term “inhibitor of Ras oncogenic isoforms,” such as H-Ras, K-Ras, or N-Ras, as used herein refers to compounds which target, decrease or inhibit the oncogenic activity of Ras; for example, a “farnesyl transferase inhibitor” such as L-744832, DK8G557 or R115777 (Zarnestra™). The term “telomerase inhibitor” as used herein refers to compounds which target, decrease or inhibit the activity of telomerase. Compounds which target, decrease or inhibit the activity of telomerase are especially compounds which inhibit the telomerase receptor, such as telomestatin.

The term “methionine aminopeptidase inhibitor” as used herein refers to compounds which target, decrease or inhibit the activity of methionine aminopeptidase. Compounds which target, decrease or inhibit the activity of methionine aminopeptidase include, but are not limited to, bengamide or a derivative thereof.

The term “proteasome inhibitor” as used herein refers to compounds which target, decrease or inhibit the activity of the proteasome. Compounds which target, decrease or inhibit the activity of the proteasome include, but are not limited to, Bortezomib (Velcade™) and MLN 341.

The term “matrix metalloproteinase inhibitor” or (“MMP” inhibitor) as used herein includes, but is not limited to, collagen peptidomimetic and nonpeptidomimetic inhibitors, tetracycline derivatives, e.g. hydroxamate peptidomimetic inhibitor batimastat and its orally bioavailable analogue marimastat (BB-2516), prinomastat (AG3340), metastat (NSC 683551) BMS-279251, BAY 12-9566, TAA211, MMI1270B or AAJ996.

The term “compounds used in the treatment of hematologic malignancies” as used herein includes, but is not limited to, FMS-like tyrosine kinase inhibitors, which are compounds targeting, decreasing or inhibiting the activity of FMS-like tyrosine kinase receptors (Flt-3R); interferon, 1-β-D-arabinofuransylcytosine (ara-c) and bisulfan; ALK inhibitors, which are compounds which target, decrease or inhibit anaplastic lymphoma kinase, and Bcl-2 inhibitors.

Compounds which target, decrease or inhibit the activity of FMS-like tyrosine kinase receptors (Flt-3R) are especially compounds, proteins or antibodies which inhibit members of the Flt-3R receptor kinase family, such as PKC412, midostaurin, a staurosporine derivative, SU11248 and MLN518.

The term “HSP90 inhibitors” as used herein includes, but is not limited to, compounds targeting, decreasing or inhibiting the intrinsic ATPase activity of HSP90; degrading, targeting, decreasing or inhibiting the HSP90 client proteins via the ubiquitin proteosome pathway. Compounds targeting, decreasing or inhibiting the intrinsic ATPase activity of HSP90 are especially compounds, proteins or antibodies which inhibit the ATPase activity of HSP90, such as 17-allylamino,17-demethoxygeldanamycin (17AAG), a geldanamycin derivative; other geldanamycin related compounds; radicicol and HDAC inhibitors.

The term “antiproliferative antibodies” as used herein includes, but is not limited to, trastuzumab (Herceptin™), Trastuzumab-DM1, erbitux, bevacizumab (Avastin™), rituximab (Rituxan®), PR064553 (anti-CD40) and 2C4 Antibody. By antibodies is meant intact monoclonal antibodies, polyclonal antibodies, multispecific antibodies formed from at least 2 intact antibodies, and antibodies fragments so long as they exhibit the desired biological activity.

For the treatment of acute myeloid leukemia (AML), compounds herein can be used in combination with standard leukemia therapies, especially in combination with therapies used for the treatment of AML. In particular, Compound 1 prepared by the method herein can be administered in combination with, for example, farnesyl transferase inhibitors and/or other drugs useful for the treatment of AML, such as Daunorubicin, Adriamycin, Ara-C, VP-16, Teniposide, Mitoxantrone, Idarubicin, Carboplatinum and PKC412. In some embodiments, a method is of treating AML associated with an ITD and/or D835Y mutation, comprising administering Compound 1 prepared by the method herein together with a one or more FLT3 inhibitors. In some embodiments, the FLT3 inhibitors are selected from quizartinib (AC220), a staurosporine derivative (e.g. midostaurin or lestaurtinib), sorafenib, tandutinib, LY-2401401, LS-104, EB-10, famitinib, NOV-110302, NMS-P948, AST-487, G-749, SB-1317, S-209, SC-110219, AKN-028, fedratinib, tozasertib, and sunitinib. In some embodiments, the FLT3 inhibitors are selected from quizartinib, midostaurin, lestaurtinib, sorafenib, and sunitinib.

Other anti-leukemic compounds include, for example, Ara-C, a pyrimidine analog, which is the 2′-alpha-hydroxy ribose (arabinoside) derivative of deoxycytidine. Also included is the purine analog of hypoxanthine, 6-mercaptopurine (6-MP) and fludarabine phosphate. Compounds which target, decrease or inhibit activity of histone deacetylase (HDAC) inhibitors such as sodium butyrate and suberoylanilide hydroxamic acid (SAHA) inhibit the activity of the enzymes known as histone deacetylases. Specific HDAC inhibitors include MS275, SAHA, FK228 (formerly FR901228), Trichostatin A and compounds disclosed in U.S. Pat. No. 6,552,065 including, but not limited to, N-hydroxy-3-[4-[[[2-(2-methyl-1H-indol-3-yl)-ethyl]-amino]methyl]phenyl]-2E-2-propenamide, or a pharmaceutically acceptable salt thereof and N-hydroxy-3-[4-[(2-hydroxyethyl){2-(1H-indol-3-yl)ethyl]-amino]methyl]phenyl]-2E-2-propenamide, or a pharmaceutically acceptable salt thereof, especially the lactate salt. Somatostatin receptor antagonists as used herein refer to compounds which target, treat or inhibit the somatostatin receptor such as octreotide, and SOM230. Tumor cell damaging approaches refer to approaches such as ionizing radiation. The term “ionizing radiation” referred to above and hereinafter means ionizing radiation that occurs as either electromagnetic rays (such as X-rays and gamma rays) or particles (such as alpha and beta particles). Ionizing radiation is provided in, but not limited to, radiation therapy and is known in the art. See Hellman, Principles of Radiation Therapy, Cancer, in Principles and Practice of Oncology, Devita et al., Eds., 4^{th} Edition, Vol. 1, pp. 248-275 (1993).

Also included are EDG binders and ribonucleotide reductase inhibitors. The term “EDG binders” as used herein refers to a class of immunosuppressants that modulates lymphocyte recirculation, such as FTY720. The term “ribonucleotide reductase inhibitors” refers to pyrimidine or purine nucleoside analogs including, but not limited to, fludarabine and/or cytosine arabinoside (ara-C), 6-thioguanine, 5-fluorouracil, cladribine, 6-mercaptopurine (especially in combination with ara-C against ALL) and/or pentostatin. Ribonucleotide reductase inhibitors are especially hydroxyurea or 2-hydroxy-1H-isoindole-1,3-dione derivatives.

Also included are in particular those compounds, proteins or monoclonal antibodies of VEGF such as 1-(4-chloroanilino)-4-(4-pyridylmethyl)phthalazine or a pharmaceutically acceptable salt thereof, 1-(4-chloroanilino)-4-(4-pyridylmethyl)phthalazine succinate; Angiostatin™; Endostatin™; anthranilic acid amides; ZD4190; ZD6474; SU5416; SU6668; bevacizumab; or anti-VEGF antibodies or anti-VEGF receptor antibodies, such as rhuMAb and RHUFab, VEGF aptamer such as Macugon; FLT-4 inhibitors, FLT-3 inhibitors, VEGFR-2 IgGI antibody, Angiozyme (RPI 4610) and Bevacizumab (Avastin™).

Photodynamic therapy as used herein refers to therapy which uses certain chemicals known as photosensitizing compounds to treat or prevent cancers. Examples of photodynamic therapy include treatment with compounds, such as Visudyne™ and porfimer sodium.

Angiostatic steroids as used herein refers to compounds which block or inhibit angiogenesis, such as, e.g., anecortave, triamcinolone, hydrocortisone, 11-α-epihydrocotisol, cortexolone, 17α-hydroxyprogesterone, corticosterone, desoxycorticosterone, testosterone, estrone and dexamethasone.

Implants containing corticosteroids refers to compounds, such as fluocinolone and dexamethasone.

Other chemotherapeutic compounds include, but are not limited to, plant alkaloids, hormonal compounds and antagonists; biological response modifiers, preferably lymphokines or interferons; antisense oligonucleotides or oligonucleotide derivatives; shRNA or siRNA; or miscellaneous compounds or compounds with other or unknown mechanism of action.

The Compound 1 prepared by the method herein is also useful as co-therapeutic compound for use in combination with other drug substances such as anti-inflammatory, bronchodilatory or antihistamine drug substances, particularly in the treatment of obstructive or inflammatory airways diseases such as those mentioned hereinbefore, for example as potentiators of therapeutic activity of such drugs or as a means of reducing required dosaging or potential side effects of such drugs. Compound 1 prepared by the method herein may be mixed with the other drug substance in a fixed pharmaceutical composition or it may be administered separately, before, simultaneously with or after the other drug substance. Accordingly a combination is provided of Compound 1 prepared by the method as hereinbefore described with an anti-inflammatory, bronchodilatory, antihistamine or anti-tussive drug substance, said compound and said drug substance being in the same or different pharmaceutical composition.

Suitable anti-inflammatory drugs include steroids, in particular glucocorticosteroids such as budesonide, beclamethasone dipropionate, fluticasone propionate, ciclesonide or mometasone furoate; non-steroidal glucocorticoid receptor agonists; LTB4 antagonists such LY293111, CGSO25019C, CP-195543, SC-53228, BIIL 284, ONO 4057, SB 209247; LTD4 antagonists such as montelukast and zafirlukast; PDE4 inhibitors such cilomilast (Ariflo® GlaxoSmithKline), Roflumilast (Byk Gulden), V-11294A (Napp), BAY19-8004 (Bayer), SCH-351591 (Schering-Plough), Arofylline (Almirall Prodesfarma), PD189659/PD168787 (Parke-Davis), AWD-12-281 (Asta Medica), CDC-801 (Celgene), SeICID (TM) CC-10004 (Celgene), VM554/UM565 (Vernalis), T-440 (Tanabe), KW-4490 (Kyowa Hakko Kogyo); A2a agonists; A2b antagonists; and beta-2 adrenoceptor agonists such as albuterol (salbutamol), metaproterenol, terbutaline, salmeterol fenoterol, procaterol, and especially, formoterol and pharmaceutically acceptable salts thereof. Suitable bronchodilatory drugs include anticholinergic or antimuscarinic compounds, in particular ipratropium bromide, oxitropium bromide, tiotropium salts and CHF 4226 (Chiesi), and glycopyrrolate.

Suitable antihistamine drug substances include cetirizine hydrochloride, acetaminophen, clemastine fumarate, promethazine, loratidine, desloratidine, diphenhydramine and fexofenadine hydrochloride, activastine, astemizole, azelastine, ebastine, epinastine, mizolastine and tefenadine.

Other useful combinations of compounds with anti-inflammatory drugs are those with antagonists of chemokine receptors, e.g. CCR-1, CCR-2, CCR-3, CCR-4, CCR-5, CCR-6, CCR-7, CCR-8, CCR-9 and CCR10, CXCR1, CXCR2, CXCR3, CXCR4, CXCR5, particularly CCR-5 antagonists such as Schering-Plough antagonists SC-351125, SCH—55700 and SCH-D, and Takeda antagonists such as N-[[4-[[[6,7-dihydro-2-(4-methylphenyl)-5H-benzo-cyclohepten-8-yl]carbonyl]amino]phenyl]-methyl]tetrahydro-N,N-dimethyl-2H-pyran-4-aminium chloride (TAK-770).

The structure of the active compounds identified by code numbers, generic or trade names may be taken from the actual edition of the standard compendium “The Merck Index” or from databases, e.g. Patents International (e.g. IMS World Publications).

Compound 1 prepared by the method herein may also be used in combination with known therapeutic processes, for example, the administration of hormones or radiation. In certain embodiments, a provided compound is used as a radiosensitizer, especially for the treatment of tumors which exhibit poor sensitivity to radiotherapy.

Compound 1 prepared by the method herein can be administered alone or in combination with one or more other therapeutic compounds, possible combination therapy taking the form of fixed combinations or the administration of Compound 1 prepared by the method herein and one or more other therapeutic compounds being staggered or given independently of one another, or the combined administration of fixed combinations and one or more other therapeutic compounds. Compound 1 prepared by the method herein can besides or in addition be administered especially for tumor therapy in combination with chemotherapy, radiotherapy, immunotherapy, phototherapy, surgical intervention, or a combination of these. Long-term therapy is equally possible as is adjuvant therapy in the context of other treatment strategies, as described above. Other possible treatments are therapy to maintain the patient's status after tumor regression, or even chemopreventive therapy, for example in patients at risk.

Those additional agents may be administered separately from a compound-containing composition as described herein, as part of a multiple dosage regimen. Alternatively, those agents may be part of a single dosage form, mixed together with Compound 1 prepared by the method herein in a single composition. If administered as part of a multiple dosage regime, the two active agents may be submitted simultaneously, sequentially or within a period of time from one another normally within five hours from one another.

As used herein, the term “combination,” “combined,” and related terms refers to the simultaneous or sequential administration of therapeutic agents. For example, Compound 1 prepared by the method herein may be administered with another therapeutic agent simultaneously or sequentially in separate unit dosage forms or together in a single unit dosage form. Accordingly, a single unit dosage form include Compound 1 prepared by the method herein, an additional therapeutic agent, and a pharmaceutically acceptable carrier, adjuvant, or vehicle.

The amount of both an inventive compound and additional therapeutic agent (in those compositions which comprise an additional therapeutic agent as described above) that may be combined with the carrier materials to produce a single dosage form will vary depending upon the host treated and the particular mode of administration. Preferably, compositions should be formulated so that a dosage of between 0.01-100 mg/kg body weight/day of an inventive compound can be administered.

In those compositions which comprise an additional therapeutic agent, that additional therapeutic agent and Compound 1 prepared by the method described herein may act synergistically. Therefore, the amount of additional therapeutic agent in such compositions will be less than that required in a monotherapy utilizing only that therapeutic agent. In such compositions a dosage of between 0.01-1,000 μg/kg body weight/day of the additional therapeutic agent can be administered.

The amount of additional therapeutic agent present in the compositions comprising Compound 1 prepared by the method herein will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

Compound 1 prepared by the method herein, or pharmaceutical compositions thereof, may also be incorporated into compositions for coating an implantable medical device, such as prostheses, artificial valves, vascular grafts, stents and catheters. Vascular stents, for example, have been used to overcome restenosis (re-narrowing of the vessel wall after injury). However, patients using stents or other implantable devices risk clot formation or platelet activation. These unwanted effects may be prevented or mitigated by pre-coating the device with a pharmaceutically acceptable composition comprising a kinase inhibitor. Implantable devices coated with Compound 1 are another embodiment.

In some embodiments, a medicament is provided comprising at least Compound 1 prepared by the method described herein, or a pharmaceutically acceptable salt or solvate thereof and a pharmaceutically acceptable carrier.

All features of each of the aspects of the invention apply to all other aspects mutatis mutandis.

In order that the invention described herein may be more fully understood, the following examples are set forth. It should be understood that these examples are for illustrative purposes only and are not to be construed as limiting this invention in any manner.

### EXEMPLIFICATION

### Example 1: Synthesis of Compound 1

Scheme 2 shown below exemplifies the detailed synthetic procedure to produce Compounds 6, 2, and 1.

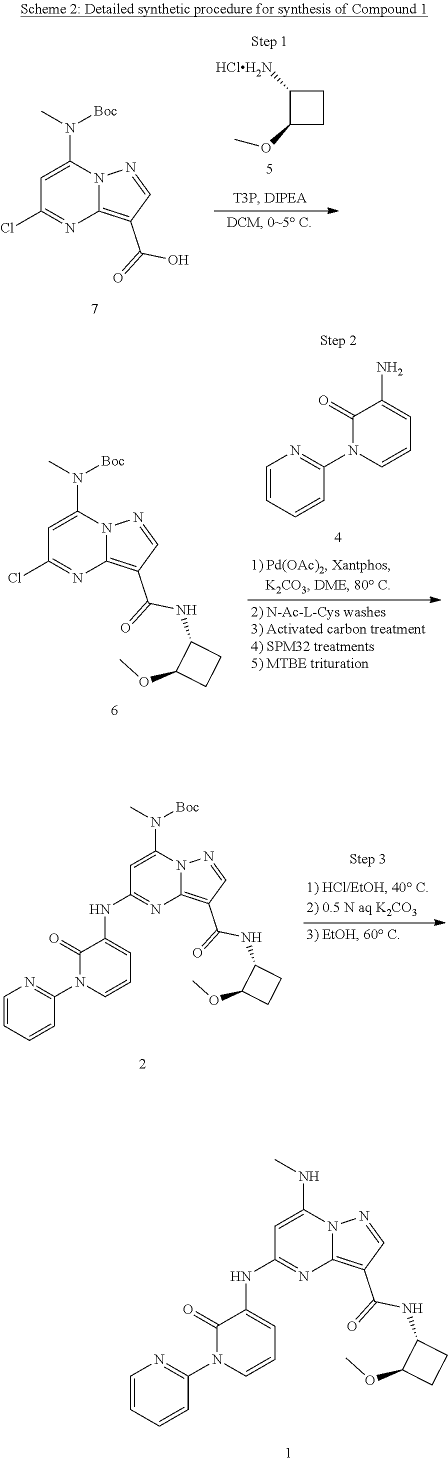

### Step 1: Synthesis of Compound 6

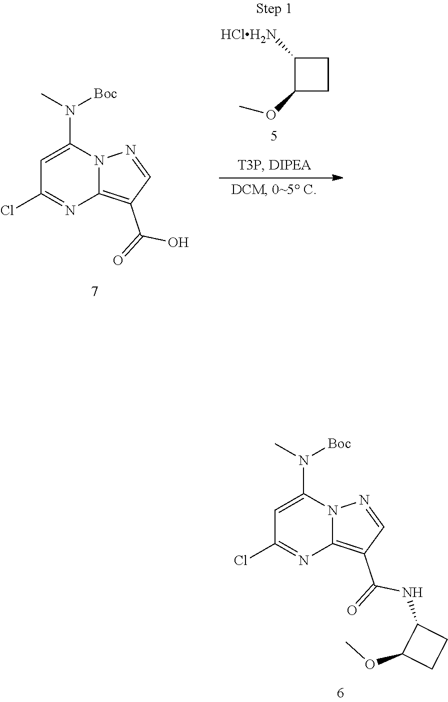

Compound 7 may be prepared as shown in the scheme below.

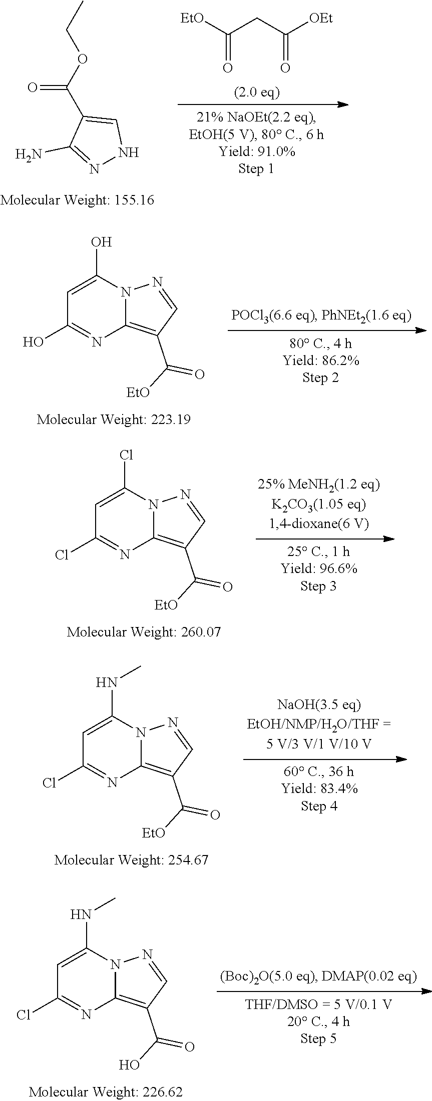 [SMILES: CCOC(=O)c1c[nH]nc1N ; CCOC(=O)CC(=O)OCC ; CCOC(=O)c1cnn2c(O)cc(O)nc12 ; CCOC(=O)c1cnn2c(Cl)cc(Cl)nc12 ; CCOC(=O)c1cnn2c(NC)cc(Cl)nc12 ; CNc1cc(Cl)nc2c(C(=O)O)cnn12]

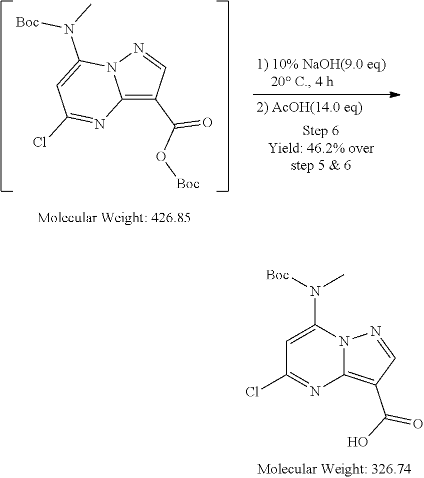

Overall yield: 29.2%. This process was used to prepare Compound 7 on a 60.0 kg scale, starting from 140.0 kg of ethyl 3-amino-1H-pyrazole-4-carboxylate. The campaign produced 29.31 kg of 7 with a chemical purity of 99.0% and 41.16 kg of 7 with a chemical purity of 98.8%.

Compound 7 (1.9 Kg, 5.82 mol, 1.00 eq), the hydrochloride salt of Compound 5 (800.9 g, 5.82 mol, 1.00 eq), DIPEA (4.5 Kg, 34.92 mol, 6.00 eq), T3P (50% in EtOAc) (7.4 Kg, 11.64 mol, 2.00 eq) were dissolved in DCM (50.3 Kg, 48.0 L, 20 vol) at 0±5° C.

After agitating at 0±5° C. for 1 h, the HPLC (30 min method, UV 242 nm) of the reaction sample showed 81% of desired product Compound 6.

After agitating the reaction at 0±5° C. for additional 1 h, the HPLC of a second reaction sample showed 82% of desired product Compound 6. The sum of starting material Compound 7 and two activated ester intermediates was 18%. At this stage, Applicant proceeded to water quench.

### Reaction Quench and Work Up

The reaction mixture was quenched with water (3.8 Kg, 3.8 L, 2 vol). The DCM was distilled and the residue was dissolved in EtOAc (68.6 Kg, 76.1 L, 40 vol). The organic layer was washed with 20% aqueous K₂CO₃ solution (33.4 Kg, 15 vol), 10% K₂CO₃ (2×19.0 Kg, 2×10 vol).

HPLC analysis of the organic layer showed 99.05% of desired product Compound 6. The sum of starting material Compound 7 and two by-product activated ester intermediates was 1%.

After passing purity test, the organic layer was sequentially washed with water (9.5 Kg, 5 vol), and brine (2×9.5 Kg, 2×5 vol). The resultant organic layer was filtered over a silica plug (5.7 Kg, 2% w/w) with 5.0 Kg sand on the top. The silica plug was washed with EtOAc (34.4 Kg, 38.0 L, 20 vol).

Trituration in n-Heptane

The combined filtrates were concentrated to about 6 L (3.2 vol). n-Heptane (7.8 Kg, 11.4 L, 6 vol) was slowly charged over a period of 30 min. The mixture was stirred at 20±5° C. for at least 2 h (actual agitation time: overnight, 15.4 h). The solid was collected through filtration and washed with n-heptane [2×5.2 Kg (2×7.6 L, 2×4 vol)].

### Drying and Packaging

After drying in a vacuum tray dryer at 43° C. for 23 h, a sample was pulled for LOD (limit of detection) test, that showed a 0.35% LOD result. The material was packaged, 1.73 Kg product Compound 6 was obtained as white solid in 72.4% yield. ¹H NMR (DMSO-d₆) ppm: 1.29 (s, 9H), 1.51 (m, 2H), 2.04 (m, 2H), 3.22 (s, 3H), 3.32 (s, 3H), 3.84 (m, 1H), 4.28 (m, 1H), 7.53 (s, 1H), 8.03 (d, 1H), 8.63 (s, 1H).

### Step 2: Synthesis of Compound 2

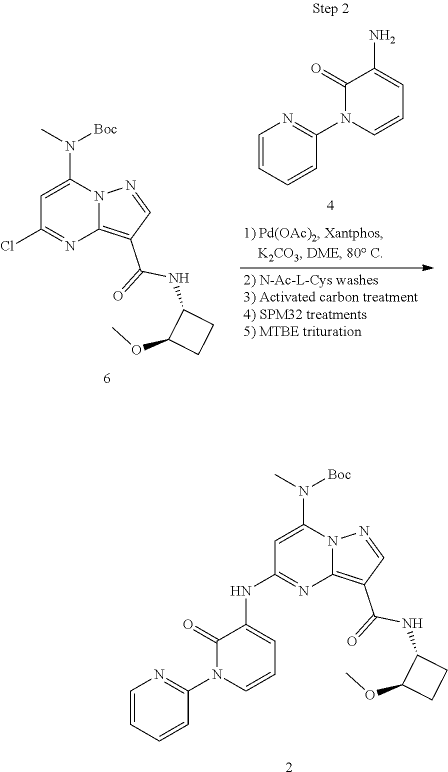 [SMILES: CO[C@@H]1CC[C@H]1NC(=O)c1cnn2c(N(C)C(=O)OC(C)(C)C)cc(Cl)nc12 ; Nc1cccn(-c2ccccn2)c1=O ; CO[C@@H]1CC[C@H]1NC(=O)c1cnn2c(N(C)C(=O)OC(C)(C)C)cc(Nc3cccn(-c4ccccn4)c3=O)nc12 ; "2"]

Compound 4 may be prepared as shown in the scheme below.

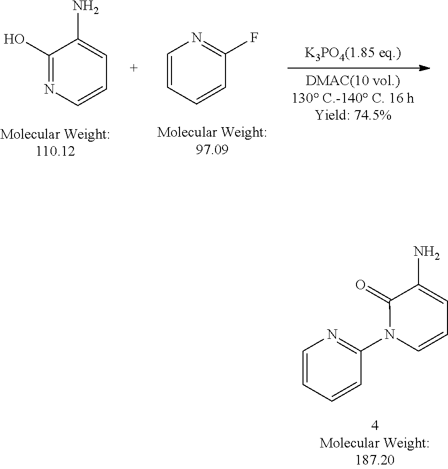 [SMILES: Nc1cccnc1O ; Fc1ccccn1 ; Nc1cccn(-c2ccccn2)c1=O]

Overall yield: 74.5%. The above route was used to deliver 3×20 kg of 4. The production batches were initiated using ˜18.0 kg (corrected by assay) of 3-aminopyridin-2-ol and ˜22.2 kg 2-fluoropyridine. The campaign produced 23.91 kg of 4 with 99.8% chromatographic purity and 22.84 kg of 4 with 99.8% chromatographic purity and 22.90 kg of 4 with 99.8% chromatographic purity respectively.

Compound 6 (1.7 Kg, 4.15 mol, 1.00 eq), Compound 4 (799.3 g, 4.27 mol, 1.03 eq), Pd(OAc)₂ (18.0 g, 0.08 mol, 0.020 eq), Xantphos (46.3 g, 0.08 mol, 0.020 eq), K₂CO₃ (1.26 Kg, 9.13 mol, 2.20 eq) in DME (26.5 Kg, 30.6 L, 18 vol) at 80±5° C. were combined in a 50 Gallon tank. The reaction was complete after 4 h at 80° C. WPLC (UV 266 nm) analysis of the reaction mixture showed 0.06% remaining Compound 6.

### Two Crops of Crude Solid

After reaction completion was confirmed, the reaction mixture was cooled to 20±5° C. and stirred at 20±5° C. for at least 30 min. The solid was filtered and washed with MTBE twice (4.2 Kg and 3.6 Kg) to provide “first crop of crude solid.” The filtrate was transferred back to the 50 gallon reactor. The mixture was concentrated until no distillates were observed. DME (3.3 Kg) and MTBE (5.6 Kg) were charged. The mixture was stirred at 20±5° C. for at least 30 min. The solid was collected through filtration and washed with MTBE (6.1 Kg) to provide the “second crop of crude solid.”

Extractions with DCM

The two crops of crude solids obtained were combined and transferred to the 50 gallon reactor. DCM (37.9 Kg) and water (28.5 Kg) were added. The mixture was stirred at 20±5° C. for at least 15 min and then allowed to settle for phase separation. The mixture was held to settle overnight (17.5 h) to eliminate emulsion.

After transferring the DCM layer, the aqueous layer was back extracted with DCM twice (12.6 Kg each time). The first back extraction was allowed to settle for 6 h and the second back extraction was allowed to settle overnight (15 h).

Optionally, the DCM/water mixture could be filtered over celite to remove the insoluble solids before phase separations. The lab runs indicated that after filtration and removal of the insoluble solids, the phase separation became very fast. Additionally, the product loss can be avoided due to an unclear phase separation.

Washes with Aq. N—Ac-L-Cys, Water, and Brine

The combined product in DCM solution was washed with 10% aq. N—Ac-L-Cysteine (NAC) three times (38.0 Kg each time). After settling for 30 min, a clean phase separation was obtained. The DCM layer was then washed successively with water (19.0 Kg) and saturated aq. NaCl solution (19.0 Kg).

### Activated Charcoal Treatment

After NAC treatment, water and brine washes, the product solution in DCM was distilled and solvent swapped to THF (twice with 5.1 Kg THF). Activated charcoal (340.0 g, 20 wt % with respect to 1.7 Kg Compound 6 as starting material) and THF (30.4 Kg, 34.2 L, 20 vol with respect to 1.7 Kg Compound 6 as starting material) were charged. The mixture was heated to 60±5° C. and agitated at 60±5° C. for at least 6 h (agitated overnight, ˜18 h). Then, the mixture was cooled to 20±5° C. and the charcoal was filtered off over celite (3.2 Kg). The celite pad was washed with THF (5.0 Kg). A sample of the THF solution was pulled. The sample solution was concentrated and tested for palladium (Pd) content analysis. Pd content was found to be <7.88 ppm.

### SPM32 Treatments

Three SPM32 treatments (570 g SPM32 for each treatment) were carried out in THF at 60±5° C. for at least 6 hours. After cooling to 20±5° C., scavenger SPM32 was filtered off over celite and the celite was washed with THF. The three treatments were as follows:

1^{st} SPM32 treatment (570 g SPM32): 60±5° C. for 18 h, Celite: 2.4 Kg, THF for celite wash: 7.3 Kg.

2^{nd} SPM32 treatment (570 g SPM32): 60±5° C. for 20.4 h, Celite: 1.5 Kg, THF for celite wash: 7.2 Kg.

3^{rd} SPM32 treatment (570 g SPM32): 60±5° C. for 21 h, Celite: 1.5 Kg, THF for celite wash: 8.1 Kg.

A sample of the THF solution was pulled. The sample solution was concentrated tested for Pd content analysis. Pd content was found to be <7.94 ppm.

### Distillation

After SPM32 treatments, the THF solution was concentrated to about 20 L in the 50 gallon tank. The THF solution was transferred to a clean drum. The tank was rinsed with THF (10.4 Kg) and the rinse was transferred to the drum. Product isolation was carried out in a 100 L reactor as follows

Distillation in 100 L reactor: The THF solution in the drum was transferred to a 100 L reactor and concentrated to about 10 L under vacuum. MTBE (14.8 Kg, 20.0 L) was charged and the mixture was concentrated to about 10 L. MTBE swap distillation was repeated two more times (14.8 Kg MTBE each time and concentrated to about 10 L).

### Trituration in MTBE

After solvent distillation (about 10 L remained), MTBE (11.1 Kg, 15 L) was charged. The mixture was heated to 50±5° C. and stirred at 50±5° C. for 1 h. After cooling to 20±5° C., the mixture was stirred at 20±5° C. overnight (19.5 h). The batch was filtered and the wet cake was washed with MTBE twice (2.8 Kg and 4.8 Kg, respectively). A sample of wet cake was pulled and analyzed via HPLC that showed a purity of 99.29%.

### Drying and Packaging

The material was dried in a vacuum tray dryer at <45° C. for at least 16 h. After drying at 43° C. for 21.7 h, a sample was pulled for LOD test that showed a LOD of 0.88%. The material was packaged. 1.5 Kg Compound 2 was obtained as an off-white solid with yield of 65.2%. ¹H NMR (DMSO-d₆)ppm: 1.33 (s, 9H), 1.50 (m, 2H), 2.10 (m, 2H), 3.21 (s, 3H), 3.25 (s, 3H), 3.77 (m, 1H), 4.34 (m, 1H), 6.49 (m, 1H), 7.18 (s, 1H), 7.55 (dd, 1H), 7.71 (dd, 1H), 7.88 (d, 1H), 8.0 (d, 1H), 8.05 (dd, 1H), 8.29 (s, 1H), 8.5 (dd, 1H), 8.65 (dd, 1H), 9.75 (s, 1H).

### Synthesis of Reagent Compound 5

Compound 5 used in the above protocol may be prepared from cyclobutanone according to the scheme and sequences shown below.

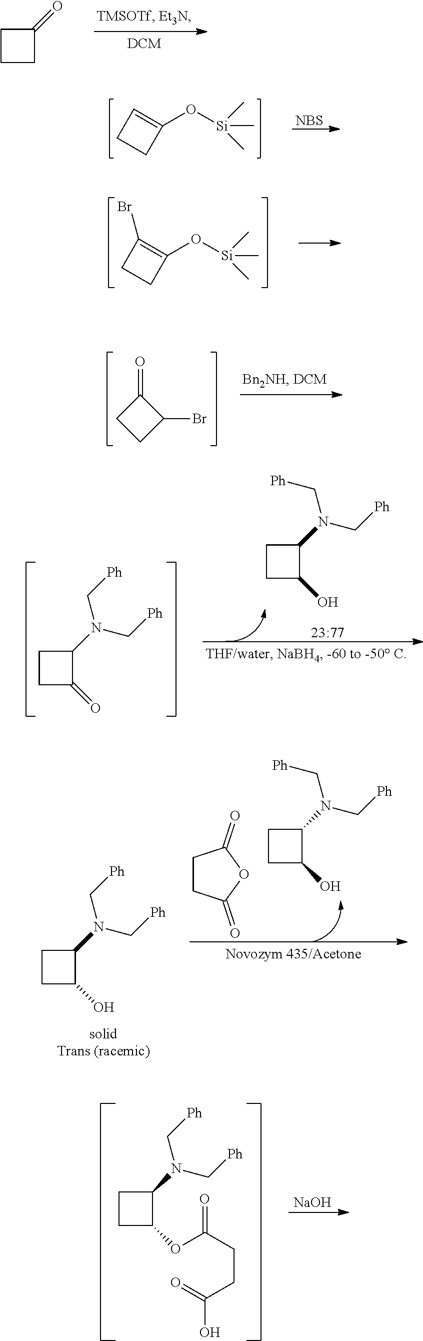

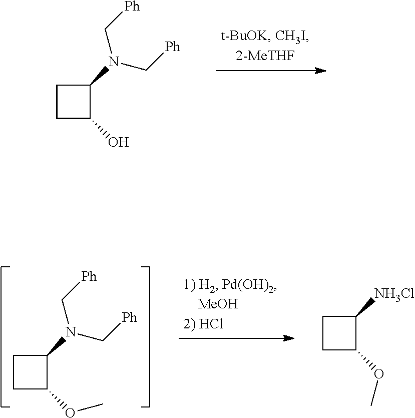

Cyclobutanone was converted to 2-bromocyclobutan-1-one by the following steps. Charged DCM (1330 kg, 10 vol.) into a 2000 L-stainless-steel reactor under N₂ protection, and followed by cyclobutanone (100 Kg, 1.0 eq.) and TEA (216.6 kg, 1.5 eq.) successively. Cooled to −15 to −25° C. Charged TMSOTf (381.1 kg, 1.2 eq.) in dropwise manner to the reactor at −15˜−25° C. for about 7 h. Stirred at −15 to −25° C. for 2 h. Charged N-bromosuccinimide (NBS) (253.3 kg, 1.0 eq.) in portions at −15 to −25° C. for about 16 h. Stirred for 2 h at −15 to −25° C. Warmed to 0-10° C., then charged soft water (200 kg, 2 vol.) and stirred for 20 min at 10-20° C. Transferred the mixture to a 3000 L-glass-line reactor, charged water (300 kg, 3.0 vol.), stirred for 20 min at 20-30° C. Separated and collected the lower organic layer. Washed organic layer with soft water (500 kg, 5 vol.). Charged 0.5 N HCl (533 kg, 5 vol.) into organic layer and then stirred at 10-20° C. for 1 h. Separated and collected the lower organic layer. Washed organic layer with soft water (500 kg, 5 vol.). Washed organic layer with 10% brine (535 kg, 5 vol.). Concentrated organic layer to 2-3 vol. under vacuum no more than 35° C. Charged DCM (665 kg, 5 vol.), then concentrated to 1-2 vol under vacuum no more than 35° C. Repeat this operation 2 times to remove water. Sample for KF (The criterion: KF ≤0.5%; Result <0.01%). Collected the residual to give light brown oil 2-bromocyclobutan-1-one (331. 8 kg of DCM solution, Assay: 32%, 106.3 kg, KF: <0.01%, Assay yield: 50%).

2-bromocyclobutan-1-one was converted to 2-(dibenzylamino)cyclobutan-1-one according to the following steps: Charged DCM (707 kg, 5 vol.) into a 3000 L-glass-line reactor under N₂ protection, followed by Bn₂NH (140 kg, 1.0 eq.) and DIPEA (115 kg, 1.25 eq.) successively. Charged 2-bromocyclobutan-1-one (106 kg, 1.0 eq.) into the reactor at 15-25° C. Stirred at 15-25° C. for 16 h. Concentrated to 4-5 volumes under vacuum no more than 40° C. Charged EtOAc (572 kg, 6 vol.), then concentrated to 4-5 volumes under vacuum no more than 40° C. Charged EtOAc (572 kg, 6 vol.). Washed with water (636 kg, 6 vol.) for once (emulsification layer was separated to aqueous layer). Washed with 2% brine (649 kg, 6 vol.) for once. Extracted EtOAc with ¹N HCl (2×636 L, 1×318 L; 2×6 vol., 1×3 vol.), combined aqueous layer. Adjusted pH of the aqueous phase to 9-10 with 20% NaOH. Charged NaCl (212 kg, 2 w/w), stirred until the solid dissolved. Extracted the aqueous layer with THE (755 kg, 8 vol.) once. Extracted the aqueous layer with THF (472 kg, 5 vol.) once again. Combined organic layer and washed with 10% brine (340 kg, 3 vol.) once. Collected the organic layer to give a light yellow solution of 2-(dibenzylamino)cyclobutan-1-one (1155 kg of THF solution, Assay: 14.98%, 173 kg of 5028-2 (Assay corrected), Purity: 93.0% (Bn₂NH: 3.0%), KF: 7.9%, Assay Yield: 92%), which was used in the next reaction sequence.

2-(dibenzylamino)cyclobutan-1-one was converted to racemic trans-2-(dibenzylamino)cyclobutan-1-ol according the following sequence: Charged the solution of 2-(dibenzylamino)cyclobutan-1-one from the previous sequence (775 kg, contained 116 kg of 2-(dibenzylamino)cyclobutan-1-one) into a 2000 L-stainless steel reactor under N₂ protection. Charged soft water (47 kg, 0.41 vol. due to THF solution contains 0.59 volume of water) and THF (266 kg, 2.68 vol.) into the reactor. Cooled to −50° C. to −60° C. Charged NaBH₄ (16.5 kg, 1.0 eq.) to the reactor in several portions at −50° C. to −60° C. Stirred for 1 h at −50° C. to −60° C. Warmed to −10° C. and charged drop wise acetone (90.5 kg, 1 vol.) to quench the reaction. Charged soft water (406 kg, 3.5 vol.) to the reaction ≤30° C. Charged MTBE (429 kg, 5 vol.) to dilute the reaction mixture. Separated the mixture and extracted water layer with MTBE (172 kg, 2 vol.), combined the organic layers. Washed the organic layer with 5% brine (300 kg, 2.5 vol.). Washed the organic phase with 20% brine (348 kg, 2.5 vol.). Concentrated the organic layer under vacuum at no more than 45° C. to 220 L (1.9 vol., target: 1-2 vol.) Charged n-heptane (6.0 vol.). Charged n-heptane (3.0 vol.). Concentrated under vacuum at no more than 45° C. to 4-5 vol. Charged n-heptane (3.0 vol.). Stirred at 15-25° C. until a large amount of solid precipitated (no less than 3 h). Heated up to 40° C.-45° C., stirred until the material (stuck on the wall of reactor) to the system, then charged n-heptane (2.0 vol.). Cooled to 10-20° C., stirred for no less than 3 h. Filtered and washed the filter cake with n-heptane (2.0 vol.). Dried under vacuum below 35° C., until KF≤0.5%, then Sampled for LOD and report the result. Collected the product to give an off-white solid racemic trans-2-(dibenzylamino)cyclobutan-1-ol (66.0 kg, P: 98.7% (Bn₂NH: 0.51%, Cis: 0.56%), Isolated Yield: 56.4%).

Racemic trans-2-(dibenzylamino)cyclobutan-1-ol was enzymatically chirally resolved according to the following sequence: Charged Acetone (1422 kg, 20 vol.) to a 3000 L reactor under N₂ protection. Stirred for 20 min and sample for KF (The criterion: KF≤0.5%, the result: KF=0.30%). Charged Racemic racemic trans-2-(dibenzylamino)cyclobutan-1-ol (95.8 kg, 1.0 eq.) to the reactor under N₂ protection. Charged Novozymes 435 lipase enzyme (18.84 kg, 0.2 w/w) to the reactor underN₂ protection. Stirred for 0.5 h at 20-25° C. Charged succinic anhydride (23.3 kg, 0.65 eq.) to the reactor at 20-25° C. under N₂ protection. Stirred for 26 h at 20-25° C. Filtered and washed the filter cake with MTBE (1 vol.) for 3 times. Charged water (1.0 eq.) into the filtrate. Collected all filtrate and concentrated under vacuum below 40° C. to 1-3 volumes. Charge MTBE (5.0 vol.) to the residual and concentrate under vacuum below 40° C. to 1-3 vol. Repeat the procedure twice. Diluted the residual with MTBE (10 vol.) and 2.5% aq. K₂CO₃ (6 vol.), then stirred for 0.5 h at 10-15° C. Separated and extracted the MTBE phase with 2.5% aq. K₂CO₃ (2*6 vol.) at 10-15° C. to ensure no ester into MTBE. Combined all aqueous phase and washed with MTBE (2*2 vol.) (Note: the solid at the interface was separated into aqueous phase). Charged solid NaOH (4.0 eq.) in portions into the aqueous phase below 10° C. Stirred for over 4 h at 15-20° C. Extracted the reaction mixture with MTBE (5.0 vol.) once. Extracted the reaction mixture with MTBE (3.0 vol.) once again (the solid at the interface was separated into aqueous phase). Combined all MTBE phases and washed with water (2*2 vol.). Concentrated the MTBE phase under vacuum below 40° C. to 2-2.5 vol. Charged heptane (5 vol.) into residual and concentrated system under vacuum below 40° C. to 3-3.5 vol. Charged heptane (5 vol.) into residual and concentrated system under vacuum below 40° C. to 3.5-4 vol. Charged heptane to (5 vol.) into residual and stirred for 6 h at 15-25° C. Filtered and washed the filter cake with n-heptane (0.5 vol.) once. Collected filter cake and dried the cake under vacuum, until KF ≤50.5%, then Sampled for LOD and report the result. Collected the product to give an off-white chirally resolved solid (1R,2R)-2-(dibenzylamino)cyclobutan-1-ol. (61.93 kg, KF: 0.1%, LOD: 0.75%, HPLC purity: 99.9%, Chiral purity: 99.7%, Isolated yield: 33.5%).

(1R,2R)-2-(dibenzylamino)cyclobutan-1-ol was converted to (1R,2R)—N,N-dibenzyl-2-methoxycyclobutan-1-amine according the the following sequence: Charged the 2-MeTHF (588 kg, 12.0 vol.) to a 2000 L-glass-line reactor under N₂ protection. Stirred for 15 min. Charged the (1R,2R)-2-(dibenzylamino)cyclobutan-1-ol (57.0 kg, 1.0 eq.) to the reactor. Stirred for 20 min to give a clear solution. Cooled reaction system to −5 to 0° C. Charged t-BuOK (33.52 kg, 1.4 eq.) in several portions into the reactor at 0±5° C. Stirred for 2 h at 0-5° C. Charged CH₃I (42.35 kg, 1.4 eq.) drop wise into the reactor at 0±5° C. Stirred for 4 h at 5±5° C. Charged soft water (5.0 vol.) at 0-30° C., and stirred for 30 min. Separated and collected the organic layer. Extracted aqueous layer with MTBE (3 vol.). Combined the organic phase and washed with soft water (2*2.5 vol.). Washed with brine (2.5 vol.) (The emulsion layer was collected separately as washing with brine). Extracted the emulsion layer with MTBE (2 vol.). Combined the organic layers. Filtered through the activated carbon filter. Concentrated the organic phase to 4-6 vol. under vacuum no more than 50° C. Charged methanol (10.0 vol.) to the reactor, concentrated to 4-6 vol. under vacuum no more than 55° C. (Jacket temperature). Charged methanol (10.0 vol.) to the reactor, concentrated to 4-6 vol. under vacuum no more than 55° C. (Jacket temperature). Collected the concentrated residue to give a solution of (1R,2R)—N,N-dibenzyl-2-methoxycyclobutan-1-amine in MeOH, with HPLC purity: 99.9%.

Compound 5 was prepared from (1R,2R)—N,N-dibenzyl-2-methoxycyclobutan-1-amine according to the following sequence: Charged the solution of (1R,2R)—N,N-dibenzyl-2-methoxycyclobutan-1-amine (30.0 kg, counted with 100% conversion rate) in MeOH (about 150 L, 5 vol., from previous sequence) into a 1000 L-autoclave under N₂ atmosphere, then charged MeOH (14 vol.). Charged the wet 20% Pd(OH)₂/C (50% water content, 3.0 kg, 0.1 w/w, based on wet weight) to the autoclave under N₂ atmosphere, then rinsed feeding port with MeOH (1.0 vol.). Stirred and charged the H₂ (1.0 MPa) into the autoclave. Stirred about 24 h at 20-30° C. Filtered and washed the filter cake with MeOH (3*1.0 vol.) under N₂ atmosphere. Collected the filtrate. Charged 4.0 N HCl(g) in MeOH (2.0 eq.) to the filtrate at 20-30° C. in drop-wise. Stirred for 2 h at 20-30° C. Combined another batch. Concentrated the reaction mixture to 3.5-4.5 vol. Charged toluene (5.0 vol.), then concentrated the reaction mixture to 3.5-4.5 vol. Repeat the process once more to remove water in the mixture. Sample for KF (the criterion: 0.10%, result, 0.07%). Charged MTBE (5.0 vol.), then concentrated the reaction mixture to 5.5-6.5 vol, then charged MTBE (1 vol.). Stirred for 3 h at 15-25° C. Filtered and washed the cake with MTBE (2 vol.). Collected the filter cake and dried for 16 h under vacuum at 40-50° C. Collected the cake to give light pink solid Compound 5 (26.92 kg, Purity: 98.1 (GC), Chiral purity: 99.9% (by deriving), KF: 0.1%, The isolated yield: 91.8%).

### Step 3: Synthesis of Compound 1

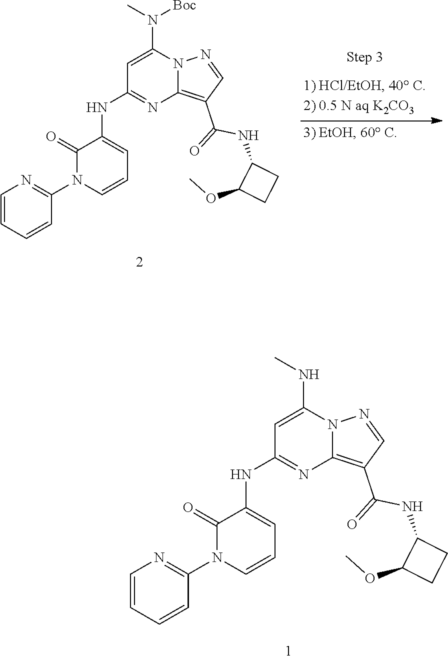 [SMILES: CO[C@@H]1CC[C@H]1NC(=O)c1cnn2c(N(C)C(=O)OC(C)(C)C)cc(Nc3cccn(-c4ccccn4)c3=O)nc12 ; "2" ; CNc1cc(Nc2cccn(-c3ccccn3)c2=O)nc2c(C(=O)N[C@@H]3CC[C@H]3OC)cnn12 ; "1"]

Compound 2 (1.5 Kg, 2.68 mol, 1.00 eq) in 3.3 M HCl/EtOH solution (19.1 Kg, 30.6 L, 15 vol) at 40±5° C. was charged into a 100 L reactor. The reaction was complete after 21 h at 40° C. HPLC (UV 266 nm) analysis of the reaction mixture showed 99.55% product Compound 1. No peak of starting material Compound 2 was detected in the HPLC. The major impurity peak (0.32%) of the reaction mixture was at 6.932 min (RRT: 0.454). The impurity peak also showed up in the HPLC of the use test reaction (0.29%, RRT 0.47) and the impurity was completely purged after Compound 1 HCl salt filtration.

Filtration of HCl salt

Once the reaction completion was confirmed, the mixture was cooled to 20±5° C. and stirred at 20±5° C. for at least 30 min. The Compound 1 HCl salt was collected through filtration. The filtration was very slow and it took 5.6 h to filter. The wet cake was washed with EtOH (first wash: 1.8 Kg, second wash: 0.8 Kg).

Neutralization with Aqueous K₂CO₃

The Compound 1 HCl salt was suspended in aqueous 0.5 N K₂CO₃ solution (30.0 Kg). The mixture was agitated at 20±5° C. for at least 1 h and a pH of 1.85 was observed. Additional amount of aqueous 0.5 N K₂CO₃ solution (42.1 Kg) was charged until a pH of >10 was observed (actual pH: 10.05). The mixture was agitated at 20±5° C. for at least 12 h (actual agitation time: 17 h). The solid was collected through filtration. The filtration was very slow and it took 5.9 h to filter. The wet cake was washed twice with water (first wash: 3.0 Kg, second wash: 4.5 Kg). The wet cake was put back to the 100 L reactor and re-slurried in water (17.0 Kg) for 1 h. The solid was collected through filtration. The wet cake was washed twice with water (first wash: 4.5 Kg, second wash: 1.1 Kg) and EtOH (1.2 Kg). The wet cake was dried in a tray dryer at <60° C. for at least 16 h.

Dryness test (1): After drying at 58° C. for 16.4 h, a sample was pulled for KF test for information only (KF result: 12.55%).

Dryness test (2): After drying at 58° C. for an additional 23.2 h (total: 39.6 h), a second sample was pulled for KF test for information only (KF result: 1.12%). This operation was done with a deviation (Deviation Report #19-111). The material was packaged and 1.14 Kg Compound 1 was obtained in 92.7% yield (before form transformation).

### Form Conversion

The dried solid Compound 1 (1.14 Kg) was transferred to a 22 L glass reactor and EtOH (8.99 Kg, 11.4 L, 10 vol, with respect to 1.14 Kg solid) was added. The mixture was agitated at 60±5° C. for 3 h. After cooling to 20±5° C. and agitating at 20±5° C. for 9.5 h, a sample was pulled and XRPD analysis showed a crystalline substance.

Solid was collected through filtration. The filter cake was transferred to a lined trays and dried in a tray dryer at <60° C. for at least 16 h. After drying at 57° C. for 92.7 h, a sample was pulled for LOD test that showed LOD of 0.20%. The material was considered to be dry and packaged. 1.1 Kg Compound 1 was obtained as an off-white solid with 96.5% recovery. The overall yield for Step 3 including reaction, neutralization and form conversion was 89.4%.

### NMR Peak Assignments for Compound 1

### Numbering System for NMR Peak Assignments:

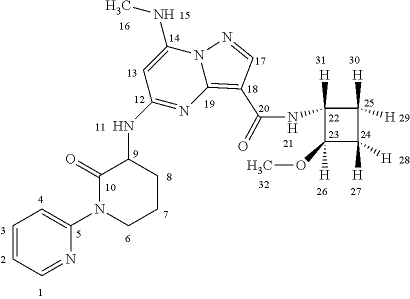

TABLE 1
Assignment of Each Signal in ¹H-NMR Spectrum
and the same ¹³C-NMR Spectrum of Compound 1

| ¹³C Atom | | | ¹H Atom | | |
|---|---|---|---|---|---|
| Position | ¹³C Chemical | Carbon | Position | ¹H Chemical | |
| Numbering | Shift (ppm) | Type | Numbering | Shift (ppm) | Multiplicity |
| 1 | 149.5 | CH | 1 | 8.65 | d |
| 2 | 124.4 | CH | 2 | 7.52 | dd |
| 3 | 138.8 | CH | 3 | 8.06 | m |
| 4 | 122.2 | CH | 4 | 7.85 | d |
| 5 | 152.3 | q | | | |
| 6 | 129.1 | CH | 6 | 7.63 | d |
| 7 | 105.9 | CH | 7 | 6.45 | t |
| 8 | 121.6 | CH | 8 | 8.35 | d |
| 9 | 131.0 | q | | | |
| 10 | 157.7 or 161.3 | carbonyl | | | |
| 11 | | | 11 | 9.01 | s |
| 12 | 156.3 | q | | | |
| 13 | 76.0 | CH | 13 | 6.23 | s |
| 14 | 146.3 | q | | | |
| 15 | | | 15 | 7.93 | q |
| 16 | 28.7 | CH3 | 16 | 2.92 | s |
| 17 | 144.0 | CH | 17 | 8.22 | s |
| 18 | 102.2 | q | | | |
| 19 | 148.4 | q | | | |
| 20 | 157.7 or 161.3 | carbonyl | | | |
| 21 | | | 21 | 8.03 | m |
| 22 | 50.5 | CH | | | |
| 23 | 81.9 | CH | | | |
| 24 | 22.5 | CH2 | | | |
| 25 | 21.3 | CH2 | | | |
| | | | 26 | 3.73 | m |
| | | | 27 | 1.52 | m |
| | | | 28 | 2.08 | m |
| | | | 29 | 1.49 | m |
| | | | 30 | 2.15 | m |
| | | | 31 | 4.34 | m |
| 32 | 55.4 | CH3 | 32 | 3.22 | s |

TABLE 2
Characteristic Absorption Bands and their
Assignment in the IR Spectrum of Compound 1

| Absorption Band | Assignment |
|---|---|
| 3350 cm⁻¹ | NH |
| 3283 cm⁻¹ | NH |
| 1647 cm⁻¹ | Amide or 2-pyridone carbonyl |

While Applicant has described a number of embodiments, it is apparent that our basic examples may be altered to provide other embodiments that utilize the compounds and methods described. Therefore, it will be appreciated that the scope of the invention is to be defined by the appended claims rather than by the specific embodiments that have been represented by way of example.

We claim:

1. A method for preparing Compound 1: 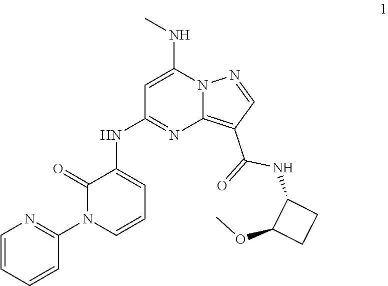 [SMILES: CNc1cc(Nc2cccn(-c3ccccn3)c2=O)nc2c(C(=O)N[C@@H]3CC[C@H]3OC)cnn12]
or a pharmaceutically acceptable salt or solvate thereof, the method comprising deprotecting a compound of formula I: 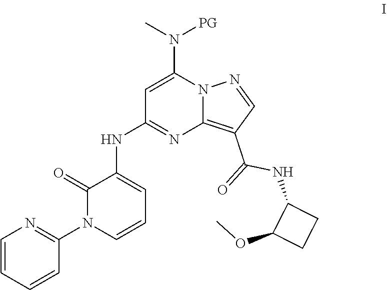
or a pharmaceutically acceptable salt thereof, wherein PG is a suitable amino protecting group.

2. The method of claim 1, further comprising the steps of:
a) cross-coupling a compound of formula II: 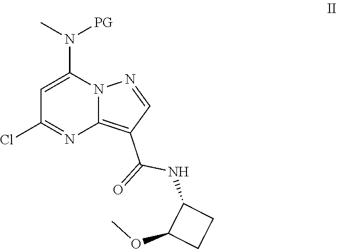
or a pharmaceutically acceptable salt thereof, wherein PG is a suitable amino protecting group; with Compound 4: 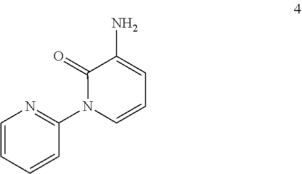 [SMILES: Nc1cccn(-c2ccccn2)c1=O]
or a pharmaceutically acceptable salt thereof, to produce a compound of formula I; and
b) deprotecting the compound of formula I: 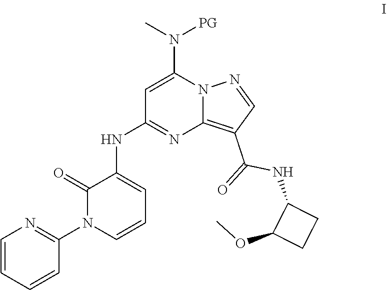
or a pharmaceutically acceptable salt thereof.

3. The method of claim 2, further comprising the steps of:
a) amidating a compound of formula III: 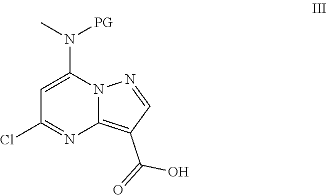
or a pharmaceutically acceptable salt thereof, wherein PG is a suitable amino protecting group; with Compound 5: 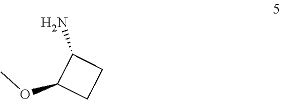
or a pharmaceutically acceptable salt thereof,
to produce the compound of formula II;
b) cross-coupling the compound of formula II: 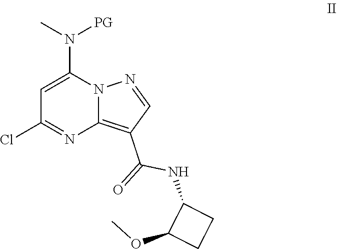
or a pharmaceutically acceptable salt thereof, wherein PG is a suitable amino protecting group; with Compound 4: 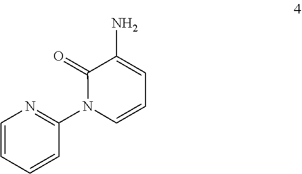 [SMILES: Nc1cccn(-c2ccccn2)c1=O]
to produce the compound of formula I; and
c) deprotecting the compound of formula I: 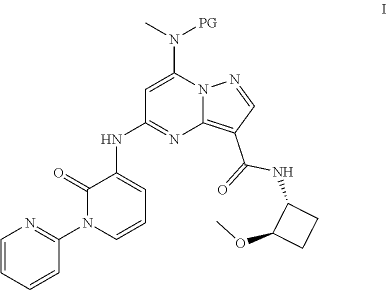
or a pharmaceutically acceptable salt thereof, to produce Compound 1.

4. The method of any one of claims 1-3, wherein PG is t-butyloxycarbonyl (BOC), ethyloxycarbonyl, methyloxycarbonyl, trichloroethyloxycarbonyl, allyloxycarbonyl (Alloc), benzyloxocarbonyl (CBZ), allyl, benzyl (Bn), fluorenylmethylcarbonyl (Fmoc), acetyl, chloroacetyl, dichloroacetyl, trichloroacetyl, trifluoroacetyl, phenylacetyl, or benzoyl.

5. The method of any one of claims 1-4, wherein PG is t-butyloxycarbonyl (BOC).

6. The method of any one of claims 1-5, wherein the compound of formula I is Compound 2: 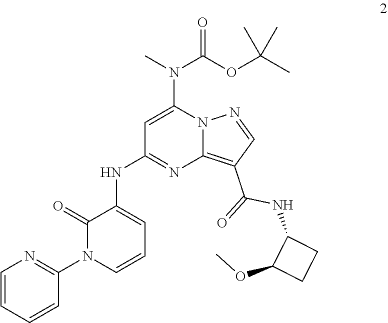 [SMILES: CO[C@@H]1CC[C@H]1NC(=O)c1cnn2c(N(C)C(=O)OC(C)(C)C)cc(Nc3cccn(-c4ccccn4)c3=O)nc12]
or a pharmaceutically acceptable salt thereof.

7. The method of any one of claims 2-6, wherein the compound of formula II is Compound 6: 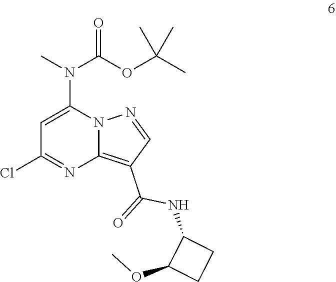 [SMILES: CO[C@@H]1CC[C@H]1NC(=O)c1cnn2c(N(C)C(=O)OC(C)(C)C)cc(Cl)nc12]
or a pharmaceutically acceptable salt thereof.

8. The method of any one of claims 3-7, wherein the compound of formula III is Compound 7: 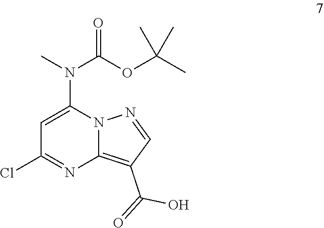 [SMILES: CN(C(=O)OC(C)(C)C)c1cc(Cl)nc2c(C(=O)O)cnn12]
or a pharmaceutically acceptable salt thereof.

9. A method of preparing a compound of formula I: 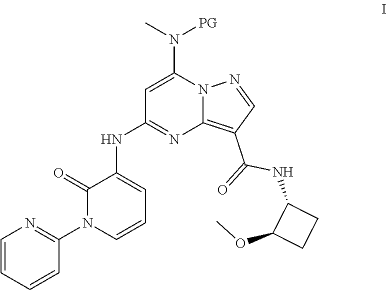
or a pharmaceutically acceptable salt thereof, wherein PG is a suitable amino protecting group, the method comprising cross-coupling a compound of formula III: 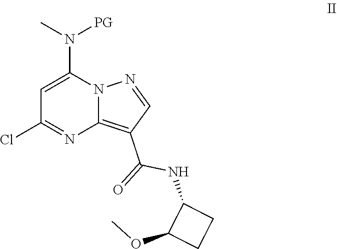
or a pharmaceutically acceptable salt thereof, with Compound 4: 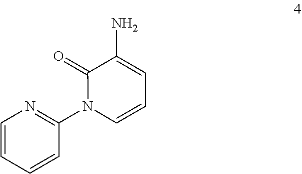 [SMILES: Nc1cccn(-c2ccccn2)c1=O]
or a pharmaceutically acceptable salt thereof.

10. The method of claim 9, further comprising:
a) amidating a compound of formula III: 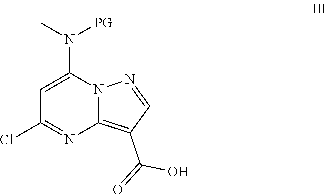
or a pharmaceutically acceptable salt thereof, with Compound 5: 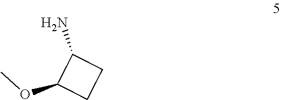
or a pharmaceutically acceptable salt thereof;
to produce the compound of formula II; and
b) cross-coupling the compound of formula II: 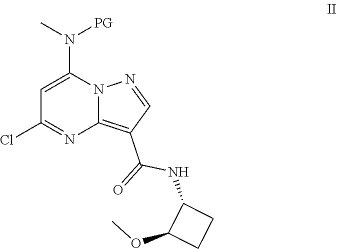
or a pharmaceutically acceptable salt thereof, with Compound 4: 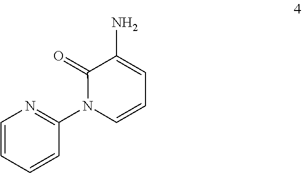 [SMILES: Nc1cccn(-c2ccccn2)c1=O]
or a pharmaceutically acceptable salt thereof,
to produce the compound of formula I.

11. A method of preparing a compound of formula II: 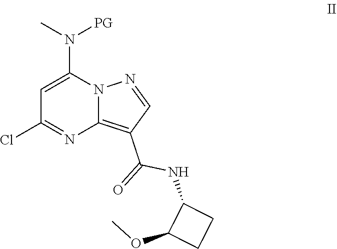
or a pharmaceutically acceptable salt thereof, wherein PG is a suitable amino protecting group; the method comprising amidating a compound of formula III: 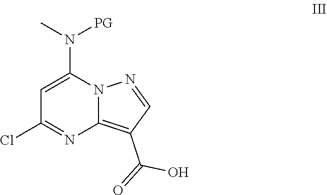
or a pharmaceutically acceptable salt thereof, with Compound 5: 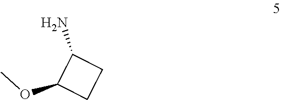
or a pharmaceutically acceptable salt thereof; to produce the compound of formula II.

12. The method of any one of claims 9-11, wherein PG is t-butyloxycarbonyl (BOC), ethyloxycarbonyl, methyloxycarbonyl, trichloroethyloxycarbonyl, allyloxycarbonyl (Alloc), benzyloxocarbonyl (CBZ), allyl, benzyl (Bn), fluorenylmethylcarbonyl (Fmoc), acetyl, chloroacetyl, dichloroacetyl, trichloroacetyl, trifluoroacetyl, phenylacetyl, or benzoyl.

13. The method of any one of claims 9-12, wherein PG is t-butyloxycarbonyl (BOC).

14. The method of claim 9 or 10, wherein the compound of formula I is Compound 2: 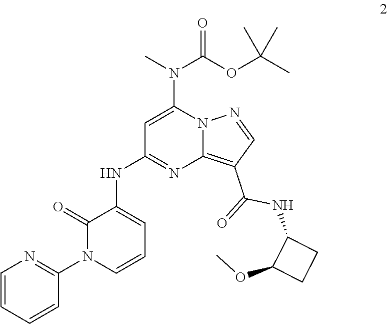 [SMILES: CO[C@@H]1CC[C@H]1NC(=O)c1cnn2c(N(C)C(=O)OC(C)(C)C)cc(Nc3cccn(-c4ccccn4)c3=O)nc12]
or a pharmaceutically acceptable salt thereof.

15. The method of any one of claims 9-14, wherein the compound of formula II is Compound 6: 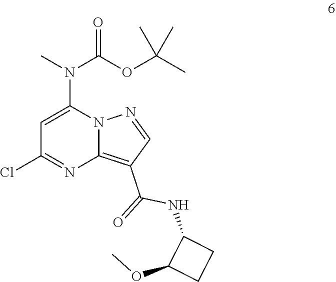 [SMILES: CO[C@@H]1CC[C@H]1NC(=O)c1cnn2c(N(C)C(=O)OC(C)(C)C)cc(Cl)nc12]
or a pharmaceutically acceptable salt thereof.

16. The method of any one of claims 10-15, wherein the compound of formula III is Compound 7: 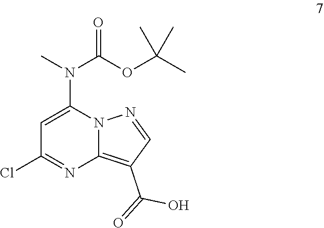 [SMILES: CN(C(=O)OC(C)(C)C)c1cc(Cl)nc2c(C(=O)O)cnn12]
or a pharmaceutically acceptable salt thereof.

17. Compound 1, or a pharmaceutically acceptable salt or solvate thereof, produced by the method of any one of claims 1-8:

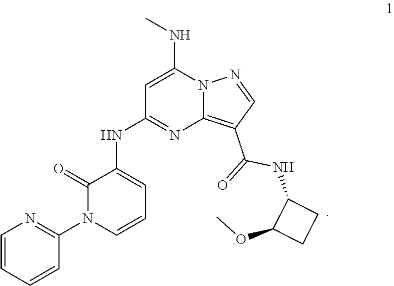 [SMILES: CNc1cc(Nc2cccn(-c3ccccn3)c2=O)nc2c(C(=O)N[C@@H]3CC[C@H]3OC)cnn12]

18. A compound of Formula I: 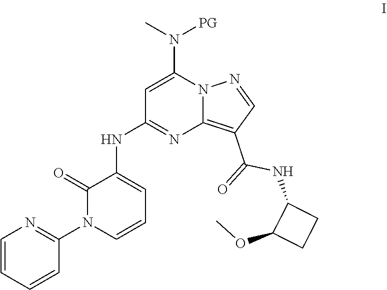
or a pharmaceutically acceptable salt or solvate thereof, wherein:
PG is a suitable amino protecting group.

19. The compound of claim 18, wherein the compound of formula I is Compound 2: 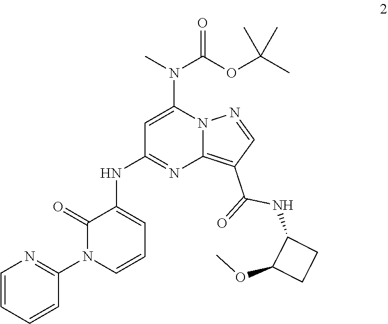 [SMILES: CO[C@@H]1CC[C@H]1NC(=O)c1cnn2c(N(C)C(=O)OC(C)(C)C)cc(Nc3cccn(-c4ccccn4)c3=O)nc12]
or a pharmaceutically acceptable salt thereof.

20. A compound of formula II: 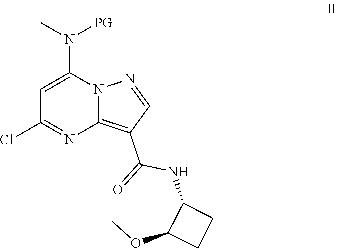
or a pharmaceutically acceptable salt or solvate thereof, wherein:
PG is a suitable amino protecting group.

21. The compound of claim 20, wherein the compound of formula II is Compound 6: 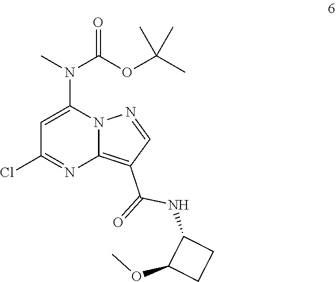 [SMILES: CO[C@@H]1CC[C@H]1NC(=O)c1cnn2c(N(C)C(=O)OC(C)(C)C)cc(Cl)nc12]
or a pharmaceutically acceptable salt or solvate thereof.

22. The compound of claim 18 or 20, wherein PG is t-butyloxycarbonyl (BOC), ethyloxycarbonyl, methyloxycarbonyl, trichloroethyloxycarbonyl, allyloxycarbonyl (Alloc), benzyloxocarbonyl (CBZ), allyl, benzyl (Bn), fluorenylmethylcarbonyl (Fmoc), acetyl, chloroacetyl, dichloroacetyl, trichloroacetyl, trifluoroacetyl, phenylacetyl, or benzoyl.

23. The compound of any one of claims 18, 20, or 22, wherein PG is t-butyloxycarbonyl (BOC).

24. A method of inhibiting a TYK2 protein kinase, or a mutant thereof, in a patient comprising administering to the patient a therapeutically effective amount of Compound 1: 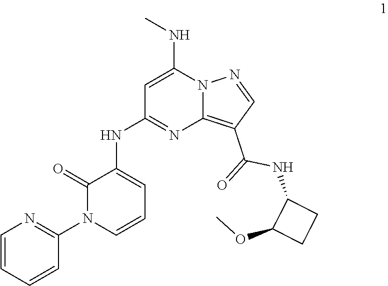 [SMILES: CNc1cc(Nc2cccn(-c3ccccn3)c2=O)nc2c(C(=O)N[C@@H]3CC[C@H]3OC)cnn12]
or a pharmaceutically acceptable salt or solvate thereof, wherein Compound 1 is produced by the method of any one of claims 1-8.
